(19) United States
(12) Patent Application Publication
(10) Pub. No.: US 2025/0243553 A1
(43) Pub. Date: Jul. 31, 2025

(54) METHODS FOR COLON CANCER DETECTION AND TREATMENT

(71) Applicant: Liquid Biopsy Research LLC, Charlestown (KN)

(72) Inventors: Irvin Mark MODLIN, Woodbridge, CT (US); Mark KIDD, New Haven, CT (US); Ignat DROZDOV, Stratford Upon Avon (GB)

(21) Appl. No.: 19/182,871

(22) Filed: Apr. 18, 2025

Related U.S. Application Data
(63) Continuation of application No. 17/815,008, filed on Jul. 26, 2022, now Pat. No. 12,305,240.
Application No. 17/815,008 is a division of application No. 16/253,697, filed on Jan. 22, 2019, now Pat. No. 11,414,707.
(60) Provisional application No. 62/620,015, filed on Jan. 22, 2018.

(51) Int. Cl. C12Q 1/6886; A61K 31/496; A61K 31/513; A61K 31/7072; A61P 35/00; C07K 16/28; C12Q 1/6827; C12Q 1/686; G16B 20/20; G16B 40/00

(52) U.S. Cl. CPC C12Q 1/6886; A61K 31/496; A61K 31/513; A61K 31/7072; A61P 35/00; C07K 16/2803; C12Q 1/6827; C12Q 1/686; G16B 20/20; G16B 40/00; C12Q 2600/106; C12Q 2600/112; C12Q 2600/158

(57) ABSTRACT

The present invention is directed to methods for detecting a colon cancer, methods for determining whether a colon cancer is stable or progressive, methods for determining a risk for disease relapse, and methods for determining a response by a subject having a colon cancer to a therapy.

### CROSS-REFERENCE TO RELATED APPLICATIONS

This application is a continuation of U.S. patent application Ser. No. 17/815,008, filed Jul. 26, 2022, which is a divisional of U.S. patent application Ser. No. 16/253,697, filed Jan. 22, 2019, now U.S. Pat. No. 11,414,707, granted on Aug. 16, 2022, which claims priority to, and the benefit of, U.S. Provisional Application No. 62/620,015, filed Jan. 22, 2018. The contents of each of the aforementioned patent applications are incorporated herein by reference in their entireties.

### SEQUENCE LISTING

The Sequence Listing XML associated with this application is provided electronically in XML file format and is hereby incorporated by reference into the specification. The name of the XML file containing the Sequence Listing XML is “LBIO-004_C01US_SeqList.xml”. The XML filed is 112,631 bytes in size, and was created on Apr. 17, 2025, and is being submitted electronically via USPTO Patent Center.

### FIELD OF THE INVENTION

The present invention relates to colon cancer detection.

### BACKGROUND OF THE INVENTION

Colorectal cancer (CRC) is one of the most commonly diagnosed cancers worldwide. In the US, CRC is the second leading cause of death as it is in Europe, after lung cancer. Worldwide, it is the fourth most common cause of cancer death. Although surgical resection followed by chemotherapy is the leading treatment option, approximately half eventually die due to distant metastases. Currently, the 5-year overall survival rate of patients with primary CRC can be up to 90%, but it will be reduced to ˜50% in patients with advanced non-metastatic tumors, and can be further decreased to <10% in patients in whom the disease is resected at its earliest stages, owing to an incomplete understanding of the molecular mechanisms underpinning its pathogenesis.

Overall survival is associated with the disease stage at the time of diagnosis, suggesting that early detection of disseminated disease is of considerable significance. Consequently, the development of new diagnostic methods that better define disease stage and can better monitor disease progression is critical.

Surveillance remains a cornerstone approach to detect recurrence at an early stage and plan further therapeutic strategies. After potentially curative resection, monitoring can be undertaken through measurement of blood biomarkers and/or imaging like CT to detect asymptomatic metastatic disease earlier. Pooled data from randomized trials published from 1995 to 2016, however, identifies that a benefit from surgical treatment resulting from earlier detection of metastases, does not occur. This likely reflects the poor sensitivity of current biomarkers.

The current biomarker is carcinoembryonic antigen (CEA), a glycoprotein involved in cell adhesion that is not generally expressed in adult tissues except in heavy smokers. Its specialized sialofucosylated glycoforms serve as functional colon carcinoma L-selectin and E-selectin ligands, which may play a role in metastatic dissemination of colon carcinoma cells. CEA is principally used to monitor colorectal carcinoma treatment, to identify recurrences after surgical resection, for staging or to localize cancer spread through measurement of biological fluids. There are, however, significant limitations. While preoperative CEA levels have shown an association with (disease-free) survival, this was chiefly because it was a surrogate for metastatic presentation. Extrapolating the predictive value of preoperative CEA has, however, been shown to be of limited significance for predictions of long-term outcomes in individual cases. This has been independently supported by a prospective analysis, which identified that levels of CEA, and other biomarkers like CA19-9, does not indicate metastasis even at a time-point where clinical signs and imaging techniques has already demonstrated metastasis.

While the molecular basis for the colorectal cancer disease has been well-characterized e.g., microsatellite instability, K-RAS mutations etc., the development of diagnostic and prognostic markers e.g., in urine or stool or as circulating-free DNA that captures this information, remains nascent but have begun to be developed. Examples include measurements of methylation of septin 9, a tumor suppressor involved in cytokinesis during cellular division. This has been used to detect colon cancer; the metrics range between 60-70%. Assessment of circulating free DNA (Line 1 and Alu-based PCR) has a predictive value of 81% with a ROC of 0.86 as a diagnostic, while measurements of circulating tumor cells are also considered useful. TPS (tissue polypeptide specific antigen) can be used as a monitor of colon cancer as can TAG-72 (tumor-associated glycoprotein) but measurements of other single analytes, like CEA or CA19-9, are non-specific.

### SUMMARY OF THE INVENTION

Among other things, disclosed herein is a 14-gene expression tool for colon cancer detection.

In one aspect, the present disclosure provides a method for detecting a colon cancer in a subject in need thereof, comprising: (a) determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a predetermined cutoff value; and (e) identifying the presence of a colon cancer in the subject when the score is equal to or greater than the predetermined cutoff value or identifying the absence of a colon cancer in the subject when the score is less than the predetermined cutoff value.

In one aspect, the present disclosure provides a method for detecting a colon cancer in a subject in need thereof, the method comprising: (a) determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a first predetermined cutoff value; and (e) producing a report, wherein the report identifies the presence of a colon cancer in the subject when the score is equal to or greater than the first predetermined cutoff value or identifies the absence of a colon cancer in the subject when the score is less than the first predetermined cutoff value, wherein the first predetermined cutoff value is 50% on a scale of 0-100%.

In one aspect, the present disclosure provides a method for determining whether a colon cancer in a subject is stable or progressive, the method comprising: (a) determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a predetermined cutoff value; and (e) identifying that the colon cancer in the subject is progressive when the score is equal to or greater than the predetermined cutoff value or identifying that the colon cancer in the subject is stable when the score is less than the predetermined cutoff value.

In one aspect, the present disclosure provides a method for determining whether a colon cancer in a subject is stable or progressive, the method comprising: (a) determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a second predetermined cutoff value; and (e) producing a report, wherein the report identifies that the colon cancer is progressive when the score is equal to or greater than the second predetermined cutoff value or identifies that the colon cancer is stable when the score is less than the second predetermined cutoff value, wherein the second predetermined cutoff value is 60% on a scale of 0 to 100%.

In one aspect, a method for determining the completeness of surgery in a subject having a colon cancer, the method comprising: (a) determining the expression level of at least 14 biomarkers from a test sample from the subject after the surgery by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a predetermined cutoff value; and (e) identifying that the colon cancer in the subject is not completely removed when the score is equal to or greater than the predetermined cutoff value or identifying that the colon cancer in the subject is completely removed when the score is less than the predetermined cutoff value.

In one aspect, the present disclosure provides a method for determining the completeness of surgery in a subject having a colon cancer, the method comprising: (a) determining the expression level of at least 14 biomarkers from a test sample from the subject after the surgery by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a first predetermined cutoff value; and (e) producing a report, wherein the report identifies that the colon cancer is not completely removed when the score is equal to or greater than the first predetermined cutoff value or identifies that the colon cancer is completely removed when the score is less than the first predetermined cutoff value, wherein the first predetermined cutoff value is 50% on a scale of 0-100%.

In one aspect, the present disclosure provides a method comprising: (a) determining the expression level of at least 14 biomarkers from a test sample from a subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a predetermined cutoff value; and (e) administering a first therapy to the subject when the score is equal to or greater than the predetermined cutoff value.

In one aspect, the present disclosure provides a method for evaluating the response of a subject having a colon cancer to a first therapy, the method comprising: (1) at a first time point: (a) determining the expression level of at least 14 biomarkers from a first test sample from the subject by contacting the first test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a first score; (2) at a second time point, wherein the second time point is after the first time point and after the administration of the therapy to the subject: (a) determining the expression level of at least 14 biomarkers from a second test sample from the subject by contacting the second test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and the housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into the algorithm to generate a second score; (3) comparing the first score with the second score; and (4) identifying that the subject is responsive to the first therapy when the second score is significantly decreased as compared to the first score or identifying that the subject is not responsive to the first therapy when the second score is not significantly decreased as compared to the first score.

In one aspect, the present disclosure provides a method for evaluating the response of a subject having a colon cancer to a therapy, the method comprising: (1) at a first time point, performing the following steps that include (a) determining the expression level of at least 14biomarkers from a first test sample from the subject by contacting the first test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a first score; and (2) at a second time point, performing the following steps that include (d) determining the expression level of at least 14 biomarkers from a second test sample from the subject by contacting the second test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and the housekeeping gene; (e) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (f) inputting each normalized expression level into the algorithm to generate a second score, wherein the second time point is after the first time point and after the administration of the therapy to the subject; (3) comparing the first score with the second score; and (4) producing a report, wherein the report identifies that the subject is responsive to the therapy when the second score is significantly decreased as compared to the first score or identifies that the subject is not responsive to the therapy when the second score is not significantly decreased as compared to the first score.

In some aspects, a method of the present disclosure can further comprise continuing to administer a first therapy to a subject when a second score is significantly decreased as compared to a first score.

In some aspects, a method of the present disclosure can further comprise discontinuing administration of a first therapy to a subject when a second score is not significantly decreased as compared to a first score.

In some aspects, a method of the present disclosure can further comprise administering a second therapy to a subject when a second score is not significantly decreased as compared to a first score.

In some aspects, a second score is significantly decreased as compared to a first score when the second score is at least 25% less than the first score.

In some aspects, a predetermined cutoff value can be 50% on a scale of 0-100%. A predetermined cutoff value can be 60% on a scale of 0-100%.

In some aspects of any one of the methods disclosed herein, a housekeeping gene can be selected from the group consisting of MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, and HPRT1. For example, the housekeeping gene can be MORF4L1.

In some aspects, a method of the present disclosure can have a sensitivity greater than 85%. In some aspects, a method of the present disclosure can have a specificity of greater than 85%.

In some aspects, a biomarker can comprise RNA, cDNA, protein or any combination thereof.

In some aspects, wherein when the biomarker is RNA, the RNA can be reverse transcribed to produce cDNA, and the produced cDNA expression level can be detected.

In some aspects, a biomarker or the expression of a biomarker can be detected by forming a complex between the biomarker and a labeled probe or primer.

In some aspects, when a biomarker is protein, the protein can be detected by forming a complex between the protein and a labeled antibody.

In some aspects, when a biomarker is RNA or cDNA, the RNA or cDNA can be detected by forming a complex between the RNA or cDNA and a labeled nucleic acid probe or primer. A complex between the RNA or cDNA and the labeled nucleic acid probe or primer can be a hybridization complex.

In some aspects, a predetermined cutoff value can be derived from a plurality of reference samples obtained from subjects not having or not diagnosed with a neoplastic disease. The neoplastic disease can be colon cancer.

In some aspects, an algorithm can be XGBoost (XGB), Random Forest (RF), glmnet, cforest, Classification and Regression Trees for Machine Learning (CART), treebag, K-Nearest Neighbors (kNN), neural network (nnet), Support Vector Machine radial (SVM-radial), Support Vector Machine linear (SVM-linear), Naïve Bayes (NB), multilayer perceptron (mlp) or any combination thereof.

In some aspects, the methods of the present disclosure can further comprise administering to a subject a first therapy when a score is equal to or greater than a predetermined cutoff.

In some aspects, a first time point can be prior to the administration of a first therapy to the subject. A first time point can be after the administration of the first therapy to the subject.

In some aspects, a therapy can comprise anti-cancer therapy, surgery, chemotherapy, targeted drug therapy, radiation therapy, immunotherapy or any combination thereof.

In some aspects, surgery can comprise removing a polyp during a colonoscopy, endoscopic mucosal resection, a partial colectomy, an ostomy, removing at least one cancerous lesion from the liver, or any combination thereof.

In some aspects, chemotherapy can comprise FOLFOX, FOLFIRI, a combination of 5-FU and leucovorin, capecitabine, irinotecan, CapeOx or any combination thereof.

In some aspects, targeted drug therapy can comprise bevacizumab, cetuximab, panitumumab, regorafenib, a combination of trifluridine and tipiracil, a EGFR TKI inhibitor or any combination thereof.

In some aspects, anti-cancer therapy can comprise anti-colon cancer therapy.

In some aspects, immunotherapy can comprise pembrolizumab, nivolumab or a combination of pembrolizumab and nivolumab.

In some aspects, a test sample can be blood, serum, plasma, neoplastic tissue or any combination thereof. A reference sample can be blood, serum, plasma, non-neoplastic tissue or any combination thereof.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

FIGS. 1A-1B are graphs showing normalized gene expression of the 13 gene signature in colon mucosa (FIG. 1A) and cell lines (FIG. 1B). Gene expression was significantly (p<0.0001) elevated in colon cancer samples (n=7) compared to matched normal mucosa (n=7). Levels were ˜20-fold elevated in colon cancer tumor tissue than in normal mucosa. All genes were expressed in three different colon cancer cell lines. Levels were ˜1000× elevated compared to normal mucosa. Horizontal lines identify median normalized expression of the 13 genes.

FIGS. 2A-2B are graphs showing receiver operator curve analysis of the test set (FIG. 2A) and independent set (FIG. 2B). Each cohort included 136 cancers and 60 controls. The AUROC in the test set was 0.9 and the Youden J index was 0.71. In the independent set the AUROC was 0.86 with a Youden index of 0.6. Z-statistics ranged 11.2-15.6 and were highly significant (p<0.0001). The sensitivity and specificity of the test ranged 85-87.5% and 75-83%, respectively.

FIGS. 3A-3B are graphs showing that gene expression in the entire cohort (controls: n=120; colon cancer cases: n=272) identified levels were significantly (p<0.0001) elevated in cases (62.7±14%) versus controls (34.6±18%) (FIG. 3A). The AUROC was 0.88, p<0.0001 (FIG. 3B). Horizontal lines identify median expression of the normalized 13 gene signature (ColoTest).

FIGS. 4A-4B are graphs showing decision curve analysis (FIG. 4A) and risk analysis (FIG. 4B) for the ColoTest. This exhibited >50% standardized predictive benefit up to a risk threshold of 80%. The probit risk assessment plot identified a ColoTest score >50% was 75% accurate for predicting colon cancer in a blood sample. This was increased to >80% at a ColoTest score >60%.

FIG. 5 is a graph showing the effect of surgery on the ColoTest. Levels prior to surgery are elevated (84±6%). In those with no evidence of disease (NED) levels were reduced by surgery to 14±9% (*p=0.0001). In those with disease (D) remaining after surgery, levels remained similar to pre-surgical values (74±4%).

FIGS. 6A-6C are graphs showing ColoTest scores in stable and progressive disease. Test scores were not significantly different between those identified as stable and those with progressive disease at the time of assessment (FIG. 6A). Of the 17 with stable disease, 12 exhibited disease progression in the 3 month follow-up. Levels in those who truly had demonstrable stable disease were low (16±10%) (FIG. 6B). In those who did progress in the 3 months levels were not different to those that had progressive disease (73±16% vs. 68±25%). The AUROC for differentiating stable from progressing/progressive disease was 0.97, p<0.0001 (FIG. 6C).

FIG. 7 is a graph showing comparison of AUROC between the ColoTest and CEA for differentiating stable from progressive disease. The ColoTest was significantly more sensitive than CEA (difference in AUC: 0.18, z-statistic: 2.1, p=0.03).

FIG. 8 is a graph showing the effect of treatment on the ColoTest. Levels prior to treatment are elevated (82±9%). In those who responded to therapy with disease stabilization, levels were reduced to 14±7% (*p<0.0001). In those that exhibited disease progression because of treatment failure, levels were elevated (69±21%).

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

Colon cancer is cancer of the large intestine (colon). Symptoms of colon cancer include, but are not limited to: (a) a change in bowel habits, (b) rectal bleeding or blood in the stool, (c) persistent abdominal discomfort, such as cramps, gas or pain, (d) a feeling that the bowel doesn't empty completely, (e) weakness or fatigue, and (f) unexplained weight loss.

Described herein are methods to quantitate (score) the circulating colon cancer molecular signature with high sensitivity and specificity for purposes including, but not limited to, detecting a colon cancer, determining whether a colon cancer is stable or progressive, determining the completeness of surgery, and evaluating the response to a colon cancer therapy. Specifically, the present invention is based on the discovery that the expression levels of ADRM1, CDK4, COMT, DHCR7, HMOX2, MCM2, PDXK, POP7, S100P, SNRPA, SORD, STOML2, and UMPS, normalized by the expression level of a housekeeping gene such as MORF4L1, are elevated in subjects having colon cancers as compared to healthy subjects.

Accordingly, the present disclosure provides a method for detecting a colon cancer in a subject in need thereof, the method comprising: (a) determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a first predetermined cutoff value; and (e) identifying the presence of a colon cancer in the subject when the score is equal to or greater than the predetermined cutoff value or identifying the absence of a colon cancer in the subject when the score is less than the predetermined cutoff value.

In some aspects of the preceding method, step (e) can comprise producing a report, wherein the report identifies the presence of a colon cancer in the subject when the score is equal to or greater than the first predetermined cutoff value or identifies the absence of a colon cancer in the subject when the score is less than the first predetermined cutoff value.

In some aspects, the preceding method can further comprise administering to the subject a first therapy. The preceding method can further comprise administering to the subject a first therapy when the score is equal to or greater than the predetermined cutoff.

The present disclosure also provides a method for determining whether a colon cancer in a subject is stable or progressive, the method comprising: (a) determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a second predetermined cutoff value; and (e) identifying that the colon cancer in the subject is progressive when the score is equal to or greater than the predetermined cutoff value or identifying that the colon cancer in the subject is stable when the score is less than the predetermined cutoff value.

In some aspects of the preceding method, step (e) can comprise producing a report, wherein the report identifies that the colon cancer is progressive when the score is equal to or greater than the second predetermined cutoff value or identifies that the colon cancer is stable when the score is less than the second predetermined cutoff value.

In some aspects, the preceding method can further comprise administering to the subject a first therapy. The preceding method can further comprise administering to the subject a first therapy when the score is equal to or greater than the predetermined cutoff.

In some aspects, the method further comprises treating the subject with a progressive colon cancer with surgery, chemotherapy, targeted drug therapy, radiation therapy, immunotherapy, or a combination thereof.

The present disclosure also provides a method for determining the completeness of surgery in a subject having a colon cancer, the method comprising: (a) determining the expression level of at least 14 biomarkers from a test sample from the subject after the surgery by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a first predetermined cutoff value; and (e) identifying that the colon cancer in the subject is not completely removed when the score is equal to or greater than the predetermined cutoff value or identifying that the colon cancer in the subject is completely removed when the score is less than the predetermined cutoff value.

In some aspects of the preceding method, step (e) can comprise producing a report, wherein the report identifies that the colon cancer is not completely removed when the score is equal to or greater than the first predetermined cutoff value or identifies that the colon cancer is completely removed when the score is less than the first predetermined cutoff value.

In some aspects, the preceding method can further comprise administering to the subject a first therapy. The preceding method can further comprise administering to the subject a first therapy when the score is equal to or greater than the predetermined cutoff.

The present disclosure also provides a method comprising: (a) determining the expression level of at least 14 biomarkers from a test sample from a subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a score; (d) comparing the score with a predetermined cutoff value; and (e) administering a first therapy to the subject when the score is equal to or greater than the predetermined cutoff value.

The response of a subject having a colon cancer to a therapy can also be evaluated by comparing the scores determined by the same algorithm at different time points of the therapy. For example, the first time point can be prior to or after the administration of the therapy to the subject; the second time point is after the first time point and after the administration of the therapy to the subject. A first score is generated at the first time point, and a second score is generated at the second time point. When the second score is significantly decreased as compared to the first score, the subject is considered to be responsive to the therapy.

Accordingly, the present disclosure provides a method for evaluating the response of a subject having a colon cancer to a first therapy, the method comprising: (1) at a first time point: (a) determining the expression level of at least 14 biomarkers from a first test sample from the subject by contacting the first test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into an algorithm to generate a first score; (2) at a second time point, wherein the second time point is after the first time point and after the administration of the therapy to the subject: (a) determining the expression level of at least 14 biomarkers from a second test sample from the subject by contacting the second test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and the housekeeping gene; (b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS; (c) inputting each normalized expression level into the algorithm to generate a second score; (3) comparing the first score with the second score; and (4) identifying that the subject is responsive to the first therapy when the second score is significantly decreased as compared to the first score or identifying that the subject is not responsive to the first therapy when the second score is not significantly decreased as compared to the first score.

In some aspects of the preceding method, step (4) can comprise producing a report, wherein the report identifies that the subject is responsive to the first therapy when the second score is significantly decreased as compared to the first score or identifies that the subject is not responsive to the first therapy when the second score is not significantly decreased as compared to the first score.

In some aspects of the preceding method, the second score is significantly decreased as compared to the first score when the second score is at least about 10% less than the first score, or at least about 20% less than the first score, or at least about 25% less than the first score, at least about 40% less than the first score, at least about 50% less than the first score, or at least about 60% less than the first score, or at least about 70% less than the first score, or at least about 75% less than the first score, or at least about 80% less than the first score, or at least about 90% less than the first score, or at least about 95% less than the first score or at least about 95% less than the first score. In some aspects, when the second score is not significantly decreased as compared to the first score, the subject is considered to be not responsive to the therapy.

In some aspects of the preceding method, a first time point can be prior to the administration of a first therapy to the subject. A first time point can be after the administration of a first therapy to the subject.

In some aspects, the preceding method can further comprise continuing to administer the first therapy to the subject when the second score is significantly decreased as compared to the first score.

In some aspects, the preceding method can further comprise discontinuing administration of the first therapy to the subject when the second score is not significantly decreased as compared to the first score.

In some aspects, the preceding method can further comprise administering a second therapy to the subject when the second score is not significantly decreased as compared to the first score.

In some aspects of the methods of the present disclosure, a predetermined cutoff value can be about 50% on a scale of 0-100%. A predetermined cutoff value can be about 60% on a scale of 0-100%. A predetermine cutoff value can be about 10%, or about 20%, or about 30%, or about 40%, or about 70%, or about 80%, or about 90% on a scale of 0-100%.

In some aspects of the methods of the present disclosure, a test sample can be any biological fluid obtained from the subject. A test sample can be blood, serum, plasma, neoplastic tissue or any combination thereof. In some aspects, the test sample is blood. In some aspects, the test sample is serum. In some aspects, the test sample is plasma.

In some aspects of the methods of the present disclosure, a housekeeping gene can comprise, but is not limited to, MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, and HPRT1. In some aspects, the housekeeping gene is MORF4L1.

The methods of the present disclosure can have a sensitivity of at least about 50%, or at least about 60%, or at least about 70%, or at least about 75%, or at least about 80%, or at least about 85%, or at least about 90%, or at least about 95%, or at least about 99%. The methods of the present disclosure can have a sensitivity of greater than about 50%, or greater than about 60%, or greater than about 70%, or greater than about 75%, or greater than about 80%, or greater than about 85%, or greater than about 90%, or greater than about 95%, or greater than about 99%.

The methods of the present disclosure can have a specificity of at least about 50%, or at least about 60%, or at least about 70%, or at least about 75%, or at least about 80%, or at least about 85%, or at least about 90%, or at least about 95%, or at least about 99%. The methods of the present disclosure can have a specificity of greater than about 50%, or greater than about 60%, or greater than about 70%, or greater than about 75%, or greater than about 80%, or greater than about 85%, or greater than about 90%, or greater than about 95%, or greater than about 99%.

The methods of the present disclosure can have an accuracy of at least about 50%, or at least about 60%, or at least about 70%, or at least about 75%, or at least about 80%, or at least about 85%, or at least about 90%, or at least about 95%, or at least about 99%. The methods of the present disclosure can have an accuracy of greater than about 50%, or greater than about 60%, or greater than about 70%, or greater than about 75%, or greater than about 80%, or greater than about 85%, or greater than about 90%, or greater than about 95%, or greater than about 99%.

In some aspects of the methods of the present disclosure, a predetermined cutoff value can be derived from a plurality of reference samples obtained from subjects not having or not diagnosed with a neoplastic disease. In some aspects, the neoplastic disease can be colon cancer.

The plurality of reference samples can comprise about 2-500, 2-200, 10-100, or 20-80 reference samples. Each reference sample produces a score using the algorithm, and the first predetermined cutoff value can be an arithmetic mean of these scores. Each reference sample can be blood, serum, plasma, or non-neoplastic tissue. In some aspects, each reference sample is blood. In some aspects, each reference sample is of the same type as the test sample.

Each of the biomarkers disclosed herein may have one or more transcript variants. The methods disclosed herein can measure the expression level of any one of the transcript variants for each biomarker.

The expression level can be measured in a number of ways, including, but not limited to: measuring the mRNA encoded by the selected genes; measuring the amount of protein encoded by the selected genes; and measuring the activity of the protein encoded by the selected genes.

In some aspects of the methods of the present disclosure, a biomarker can be RNA, cDNA, protein or any combination thereof. When the biomarker is RNA, the RNA can be reverse transcribed to produce cDNA (such as by RT-PCR), and the produced cDNA expression level can be detected. The expression level of a biomarker can be detected by forming a complex between the biomarker and a labeled probe or primer. When the biomarker is RNA or cDNA, the RNA or cDNA can be detected by forming a complex between the RNA or cDNA and a labeled nucleic acid probe or primer. The complex between the RNA or cDNA and the labeled nucleic acid probe or primer can be a hybridization complex.

In some aspects of the methods of the present disclosure, gene expression can be detected by microarray analysis. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In some aspects of microarray techniques, PCR amplified inserts of cDNA clones are applied to a substrate in a dense array. In some aspects, at least 10,000 nucleotide sequences are applied to the substrate. The microarrayed genes, immobilized on the microchip at 10,000elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In some aspects of the methods of the present disclosure, the biomarkers can be detected in a biological sample using qRT-PCR. The first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

In some aspects of the methods of the present disclosure, when the biomarker is a protein, the protein can be detected by forming a complex between the protein and a labeled antibody. The label can be any label for example a fluorescent label, chemiluminescence label, radioactive label, etc. Exemplary methods for protein detection include, but are not limited to, enzyme immunoassay (EIA), radioimmunoassay (RIA), Western blot analysis and enzyme linked immunoabsorbant assay (ELISA). For example, the biomarker can be detected in an ELISA, in which the biomarker antibody is bound to a solid phase and an enzyme-antibody conjugate is employed to detect and/or quantify biomarker present in a sample. Alternatively, a western blot assay can be used in which solubilized and separated biomarker is bound to nitrocellulose paper. The combination of a highly specific, stable liquid conjugate with a sensitive chromogenic substrate allows rapid and accurate identification of samples.

In some aspects of the methods of the present disclosure, the methods described herein can have a specificity, sensitivity, and/or accuracy of at least 75%, 80%, 85%, 90%, 91%, 92%, 93%, 94%, 95%, 96%, 97%, 98%, or 99%.

In some aspects of the methods of the present disclosure, a labeled probe, a labeled primer, a labeled antibody or a labeled nucleic acid can comprise a fluorescent label.

Any algorithm that can generate a score for a sample by assessing where that sample value falls onto a prediction model generated using different techniques, e.g., decision trees, can be used in the methods disclosed herein. The algorithm analyzes the data (i.e., expression levels) and then assigns a score. In some aspects, the algorithm can be a machine-learning algorithm. Exemplary algorithms that can be used in the methods disclosed herein can include, but are not limited to, XGBoost (XGB), Random Forest (RF), glmnet, cforest, Classification and Regression Trees for Machine Learning (CART), treebag, K-Nearest Neighbors (kNN), neural network (nnet), Support Vector Machine radial (SVM-radial), Support Vector Machine linear (SVM-linear), Naïve Bayes (NB), multilayer perceptron (mlp) or any combination thereof.

In some aspects of the methods of the present disclosure, the algorithm can be XGB (also called XGBoost). XGB is an implementation of gradient boosted decision trees designed for speed and performance.

In some aspects of the methods of the present disclosure, a therapy can comprise anti-cancer therapy, surgery, chemotherapy, targeted drug therapy, radiation therapy, immunotherapy, or any combination thereof.

In some aspects of the methods of the present disclosure, surgery can comprise removing a polyp during a colonoscopy, endoscopic mucosal resection, a partial colectomy, an ostomy, removing at least one cancerous lesion from the liver, or any combination thereof.

In some aspects of the methods of the present disclosure, anti-cancer therapy can comprise anti-colon cancer therapy.

In some aspects of the methods of the present disclosure, chemotherapy can comprise FOLFOX, FOLFIRI, a combination of 5-FU and leucovorin, capecitabine, irinotecan, CapeOx or any combination thereof.

In some aspects of the methods of the present disclosure, targeted drug therapy can comprise bevacizumab, cetuximab, panitumumab, regorafenib, a combination of trifluridine and tipiracil, an EGFR TKI inhibitor or any combination thereof.

In some aspects of the methods of the present disclosure, immunotherapy can comprise pembrolizumab, nivolumab or a combination of pembrolizumab and nivolumab.

For early-stage colon cancer, a minimally invasive approach to surgery can be used to remove the cancer. For example, if the cancer is completely contained within a polyp, the polyp can be removed during a colonoscopy. Endoscopic mucosal resection can be performed to remove larger polyps. Polyps that cannot be removed during a colonoscopy may be removed using laparoscopic surgery.

If the cancer has grown into or through the colon, partial colectomy can be performed to remove the part of the colon that contains the cancer, along with a margin of normal tissue on either side of the cancer. When it's not possible to reconnect the healthy portions of the colon or rectum, an ostomy can be performed to create an opening in the wall of the abdomen from a portion of the remaining bowel for the elimination of stool into a bag that fits securely over the opening. Lymph node removal can also be performed.

For advanced colon cancer, an operation to relieve a blockage of the colon or other conditions can also be performed. In specific cases where the cancer has spread only to the liver, surgery to remove the cancerous lesion from the liver can be performed.

For chemotherapies, either the FOLFOX (5-FU, leucovorin, and oxaliplatin) or CapeOx (capecitabine and oxaliplatin) regimens are used most often, but some patients may get 5-FU with leucovorin or capecitabine alone based on their age and health needs. Irinotecan can also be used as a chemotherapeutic agent for treating a colon cancer.

Targeted drug therapies target specific malfunctions that allow cancer cells to grow. These therapies include, but are not limited to, bevacizumab, cetuximab, panitumumab, ramucirumab, regorafenib, ziv-aflibercept, a combination of trifluridine and tipiracil, and an EGFR TKI inhibitor.

Immunotherapies for colon cancer include, but are not limited to, pembrolizumab (Keytruda®) and nivolumab (Opdivo®).

The sequence information of the colon cancer biomarkers and housekeepers is shown in Table 1.

TABLE 1
Colon Cancer Biomarker/Housekeeper Sequence Information

| | | | SEQ |
|---|---|---|---|
| Gene | RefSeq | | ID |
| Name | Accession | Sequence | NO: |
| ADRM1 | NM_007002.3 | gttagagccggctgcgcggcttacggggctcaatcggcgg | 1 |
| | | cgagagcggcaggcggggcgggccgaacgcgggtttccgg | |
| | | cggggcccggcaggcgccgaggaggaagagcgagcccgga | |
| | | cggcgcctctcgaacgagtgtgggcgcgaggcaggatgac | |
| | | gacctcaggcgcgctctttccaagcctggtgccaggctct | |
| | | cggggcgcctccaacaagtacttggtggagtttcgggcgg | |
| | | gaaagatgtccctgaaggggaccaccgtgactccggataa | |
| | | gcggaaagggctggtgtacattcagcagacggacgactcg | |
| | | cttattcacttctgctggaaggacaggacgtccgggaacg | |
| | | tggaagacgacttgatcatcttccctgacgactgtgagtt | |
| | | caagcgggtgccgcagtgccccagcgggagggtctacgtg | |
| | | ctgaagttcaaggcagggtccaagcggcttttcttctgga | |
| | | tgcaggaacccaagacagaccaggatgaggagcattgccg | |
| | | gaaagtcaacgagtatctgaacaaccccccgatgcctggg | |
| | | gcgctgggggccagcggaagcagcggccacgaactctctg | |
| | | cgctaggcggtgagggtggcctgcagagcctgctgggaaa | |
| | | catgagccacagccagctcatgcagctcatcggaccagcc | |
| | | ggcctcggaggactgggtgggctgggggccctgactggac | |
| | | ctggcctggccagcttactggggagcagtgggcctccagg | |
| | | gagcagctcctcctccagctcccggagccagtcggcagcg | |
| | | gtcaccccgtcatccaccacctcttccacccgtgccaccc | |
| | | cagccccttctgctccagcagctgcctcagcaactagccc | |
| | | gagccccgcgcccagttccgggaatggagccagcacagca | |
| | | gccagcccgacccagcccatccagctgagcgacctccaga | |
| | | gcatcctggccacgatgaacgtaccagccgggccagcagg | |
| | | cggccagcaagtggacctggccagtgtgctgacgccggag | |
| | | ataatggctcccatcctcgccaacgcggatgtccaggagc | |
| | | gcctgcttccctacttgccatctggggagtcgctgccgca | |
| | | gaccgcggatgagatccagaataccctgacctcgccccag | |
| | | ttccagcaggccctgggcatgttcagcgcagccttggcct | |
| | | cggggcagctgggccccctcatgtgccagttcggtctgcc | |
| | | tgcagaggctgtggaggccgccaacaagggcgatgtggaa | |
| | | gcgtttgccaaagccatgcagaacaacgccaagcccgagc | |
| | | agaaagagggcgacacgaaggacaagaaggacgaagagga | |
| | | ggacatgagcctggactgagccacgcgccgtcctccgagg | |
| | | aactgggcgcttgcagtgcgttgcacaccctcacctccca | |
| | | cccactgattattaataaagtcttttcttttacctgccaa | |
| | | aaaaaaaaaaaaaaaa | |
| CDK4 | NM_000075.3 | cacctcctgtccgcccctcagcgcatgggtggcggtcacg | 2 |
| | | tgcccagaacgtccggcgttcgccccgccctcccagtttc | |
| | | cgcgcgcctctttggcagctggtcacatggtgagggtggg | |
| | | ggtgagggggcctctctagcttgcggcctgtgtctatggt | |
| | | cgggccctctgcgtccagctgctccggaccgagctcgggt | |
| | | gtatggggccgtaggaaccggctccggggccccgataacg | |
| | | ggccgcccccacagcaccccgggctggcgtgagggtctcc | |
| | | cttgatctgagaatggctacctctcgatatgagccagtgg | |
| | | ctgaaattggtgtcggtgcctatgggacagtgtacaaggc | |
| | | ccgtgatccccacagtggccactttgtggccctcaagagt | |
| | | gtgagagtccccaatggaggaggaggtggaggaggccttc | |
| | | ccatcagcacagttcgtgaggtggctttactgaggcgact | |
| | | ggaggcttttgagcatcccaatgttgtccggctgatggac | |
| | | gtctgtgccacatcccgaactgaccgggagatcaaggtaa | |
| | | ccctggtgtttgagcatgtagaccaggacctaaggacata | |
| | | tctggacaaggcacccccaccaggcttgccagccgaaacg | |
| | | atcaaggatctgatgcgccagtttctaagaggcctagatt | |
| | | tccttcatgccaattgcatcgttcaccgagatctgaagcc | |
| | | agagaacattctggtgacaagtggtggaacagtcaagctg | |
| | | gctgactttggcctggccagaatctacagctaccagatgg | |
| | | cacttacacccgtggttgttacactctggtaccgagctcc | |
| | | cgaagttcttctgcagtccacatatgcaacacctgtggac | |
| | | atgtggagtgttggctgtatctttgcagagatgtttcgtc | |
| | | gaaagcctctcttctgtggaaactctgaagccgaccagtt | |
| | | gggcaaaatctttgacctgattgggctgcctccagaggat | |
| | | gactggcctcgagatgtatccctgccccgtggagcctttc | |
| | | cccccagagggccccgcccagtgcagtcggtggtacctga | |
| | | gatggaggagtcgggagcacagctgctgctggaaatgctg | |
| | | acttttaacccacacaagcgaatctctgcctttcgagctc | |
| | | tgcagcactcttatctacataaggatgaaggtaatccgga | |
| | | gtgagcaatggagtggctgccatggaaggaagaaaagctg | |
| | | ccatttcccttctggacactgagagggcaatctttgcctt | |
| | | tatctctgaggctatggagggtcctcctccatctttctac | |
| | | agagattactttgctgccttaatgacattcccctcccacc | |
| | | tctccttttgaggcttctccttctccttcccatttctcta | |
| | | cactaaggggtatgttccctcttgtccctttccctacctt | |
| | | tatatttggggtccttttttatacaggaaaaacaaaacaa | |
| | | agaaataatggtcttttttttttttttaatgtttcttcct | |
| | | ctgtttggctttgccattgtgcgatttggaaaaaccactt | |
| | | ggaagaagggactttcctgcaaaaccttaaagactggtta | |
| | | aattacagggcctaggaagtcagtggagccccttgactga | |
| | | caaagcttagaaaggaactgaaattgcttctttgaatatg | |
| | | gattttaggcggggcgtggtggctcacgcctataatccca | |
| | | gcacgttgggaggccaacgcgggtggatcacctgaggtca | |
| | | ggagttcgagaccagcctgactaacatggtgaaaccctgt | |
| | | ctctactaaaaatacaaaattagtcaggcgtggtggtgca | |
| | | cacctgtaatcccagctacttgggagactgaggcaggagg | |
| | | atcgcttgaacccgggaggcagaggttgcggtgagccgag | |
| | | atcatgccattgcactccagcctgggcaacagagcaagac | |
| | | tctgtgtcaaaaaaaaaaaaagaatatagatttttaaatg | |
| | | gcaaaaaaaaaaaaaaaaaa | |
| COMT | NM_000754.3 | cggcctgcgtccgccaccggaagcgccctcctaatccccg | 3 |
| | | cagcgccaccgccattgccgccatcgtcgtggggcttctg | |
| | | gggcagctagggctgcccgccgcgctgcctgcgccggacc | |
| | | ggggcgggtccagtcccgggcgggccgtcgcgggagagaa | |
| | | ataacatctgctttgctgccgagctcagaggagaccccag | |
| | | acccctcccgcagccagagggctggagcctgctcagaggt | |
| | | gctttgaagatgccggaggccccgcctctgctgttggcag | |
| | | ctgtgttgctgggcctggtgctgctggtggtgctgctgct | |
| | | gcttctgaggcactggggctggggcctgtgccttatcggc | |
| | | tggaacgagttcatcctgcagcccatccacaacctgctca | |
| | | tgggtgacaccaaggagcagcgcatcctgaaccacgtgct | |
| | | gcagcatgcggagcccgggaacgcacagagcgtgctggag | |
| | | gccattgacacctactgcgagcagaaggagtgggccatga | |
| | | acgtgggcgacaagaaaggcaagatcgtggacgccgtgat | |
| | | tcaggagcaccagccctccgtgctgctggagctgggggcc | |
| | | tactgtggctactcagctgtgcgcatggcccgcctgctgt | |
| | | caccaggggcgaggctcatcaccatcgagatcaaccccga | |
| | | ctgtgccgccatcacccagcggatggtggatttcgctggc | |
| | | gtgaaggacaaggtcacccttgtggttggagcgtcccagg | |
| | | acatcatcccccagctgaagaagaagtatgatgtggacac | |
| | | actggacatggtcttcctcgaccactggaaggaccggtac | |
| | | ctgccggacacgcttctcttggaggaatgtggcctgctgc | |
| | | ggaaggggacagtgctactggctgacaacgtgatctgccc | |
| | | aggtgcgccagacttcctagcacacgtgcgcgggagcagc | |
| | | tgctttgagtgcacacactaccaatcgttcctggaataca | |
| | | gggaggtggtggacggcctggagaaggccatctacaaggg | |
| | | cccaggcagcgaagcagggccctgactgcccccccggccc | |
| | | ccctctcgggctctctcacccagcctggtactgaaggtgc | |
| | | cagacgtgctcctgctgaccttctgcggctccgggctgtg | |
| | | tcctaaatgcaaagcacacctcggccgaggcctgcgccct | |
| | | gacatgctaacctctctgaactgcaacactggattgttct | |
| | | tttttaagactcaatcatgacttctttactaacactggct | |
| | | agctatattatcttatatactaatatcatgttttaaaaat | |
| | | ataaaatagaaattaagaatctaaatatttagatataact | |
| | | cgacttagtacatccttctcaactgccattcccctgctgc | |
| | | ccttgacttgggcaccaaacattcaaagctccccttgacg | |
| | | gacgctaacgctaagggcggggcccctagctggctgggtt | |
| | | ctgggtggcacgcctggcccactggcctcccagccacagt | |
| | | ggtgcagaggtcagccctcctgcagctaggccaggggcac | |
| | | ctgttagccccatggggacgactgccggcctgggaaacga | |
| | | agaggagtcagccagcattcacacctttctgaccaagcag | |
| | | gcgctggggacaggtggaccccgcagcagcaccagcccct | |
| | | ctgggccccatgtggcacagagtggaagcatctccttccc | |
| | | tactccccactgggccttgcttacagaagaggcaatggct | |
| | | cagaccagctcccgcatccctgtagttgcctccctggccc | |
| | | atgagtgaggatgcagtgctggtttctgcccacctacacc | |
| | | tagagctgtccccatctcctccaaggggtcagactgctag | |
| | | ccacctcagaggctccaagggcccagttcccaggcccagg | |
| | | acaggaatcaaccctgtgctagctgagttcacctgcaccg | |
| | | agaccagcccctagccaagattctactcctgggctcaagg | |
| | | cctggctagcccccagccagcccactcctatggatagaca | |
| | | gaccagtgagcccaagtggacaagtttggggccacccagg | |
| | | gaccagaaacagagcctctgcaggacacagcagatgggca | |
| | | cctgggaccacctccacccagggccctgccccagacgcgc | |
| | | agaggcccgacacaagggagaagccagccacttgtgccag | |
| | | acctgagtggcagaaagcaaaaagttcctttgctgcttta | |
| | | atttttaaattttcttacaaaaatttaggtgtttaccaat | |
| | | agtcttattttggcttatttttaa | |
| DHCR7 | NM_001163817.1 | aatcgctgacatcatccgggggcgggcgcccctgccctgc | 4 |
| | | gggtgactccgacccctggctagagggtaggcggcgtgga | |
| | | gcagcgcgcgcaagcgaggccaggggaaggtgggcgcagg | |
| | | actttagccggttgagaaggatcaagcaggcatttggagc | |
| | | acaggtgtctagaaacttttaaggggccggttcaagaagg | |
| | | aaaagttcccttctgctgtgaaactatttggcaagaggct | |
| | | ggagggcccaatggctgcaaaatcgcaacccaacattccc | |
| | | aaagccaagagtctagatggcgtcaccaatgacagaaccg | |
| | | catctcaagggcagtggggccgtgcctgggaggtggactg | |
| | | gttttcactggcgagcgtcatcttcctactgctgttcgcc | |
| | | cccttcatcgtctactacttcatcatggcttgtgaccagt | |
| | | acagctgcgccctgactggccctgtggtggacatcgtcac | |
| | | cggacatgctcggctctcggacatctgggccaagactcca | |
| | | cctataacgaggaaagccgcccagctctataccttgtggg | |
| | | tcaccttccaggtgcttctgtacacgtctctccctgactt | |
| | | ctgccataagtttctacccggctacgtaggaggcatccag | |
| | | gagggggccgtgactcctgcaggggttgtgaacaagtatc | |
| | | agatcaatggcctgcaagcctggctcctcacgcacctgct | |
| | | ctggtttgcaaacgctcatctcctgtcctggttctcgccc | |
| | | accatcatcttcgacaactggatcccactgctgtggtgcg | |
| | | ccaacatccttggctatgccgtctccaccttcgccatggt | |
| | | caagggctacttcttccccaccagcgccagagactgcaaa | |
| | | ttcacaggcaatttcttttacaactacatgatgggcatcg | |
| | | agtttaaccctcggatcgggaagtggtttgacttcaagct | |
| | | gttcttcaatgggcgccccgggatcgtcgcctggaccctc | |
| | | atcaacctgtccttcgcagcgaagcagcgggagctccaca | |
| | | gccatgtgaccaatgccatggtcctggtcaacgtcctgca | |
| | | ggccatctacgtgattgacttcttctggaacgaaacctgg | |
| | | tacctgaagaccattgacatctgccatgaccacttcgggt | |
| | | ggtacctgggctggggcgactgtgtctggctgccttatct | |
| | | ttacacgctgcagggtctgtacttggtgtaccaccccgtg | |
| | | cagctgtccaccccgcacgccgtgggcgtcctgctgctgg | |
| | | gcctggtgggctactacatcttccgggtggccaaccacca | |
| | | gaaggacctgttccgccgcacggatgggcgctgcctcatc | |
| | | tggggcaggaagcccaaggtcatcgagtgctcctacacat | |
| | | ccgccgatgggcagaggcaccacagcaagctgctggtgtc | |
| | | gggcttctggggcgtggcccgccacttcaactacgtcggc | |
| | | gacctgatgggcagcctggcctactgcctggcctgtggcg | |
| | | gcggccacctgctgccctacttctacatcatctacatggc | |
| | | catcctgctgacccaccgctgcctccgggacgagcaccgc | |
| | | tgcgccagcaagtacggccgggactgggagcgctacaccg | |
| | | ccgcagtgccttaccgcctgctgcctggaatcttctaagg | |
| | | gcacgccctagggagaagccctgtggggctgtcaagagcg | |
| | | tgttctgccaggtccatgggggctggcatcccagctccaa | |
| | | ctcgaggagcctcagtttcctcatctgtaaactggagaga | |
| | | gcccagcacttggcaggtgtccagtacctaatcacgctct | |
| | | gttccttgcttttgccttcaagggaattccgagtgtccag | |
| | | cactgccgtattgccagcacagacggattttctctaatca | |
| | | gtgtccctggggcaggaggatgacccagtcacctttacta | |
| | | gtcctttggagacaatttacctgtattaggagcccaggcc | |
| | | acgctacactctgcccacactggtgagcaggaggtcttcc | |
| | | cacgccctgtcattaggctgcatttactcttgctaaataa | |
| | | aagtgggagtggggcgtgcgcgttatccatgtattgcctt | |
| | | tcagctctagatccccctcccctgcctgctctgcagtcgt | |
| | | gggtggggcccgtgcgccgtttctccttggtagcgtgcac | |
| | | ggtgttgaactgggacactggggagaaaggggctttcatg | |
| | | tcgtttccttcctgctcctgctgcacagctgccaggagtg | |
| | | ctctgcctggagtctgcagacctcagagaggtcccagcac | |
| | | cggctgtggcctttcaggtgtaggcaggtgggctctgctt | |
| | | cccgattccctgtgagcgcccaccctctcgaaagaatttt | |
| | | ctgcttgccctatgactgtgcagactctggctcgagcaac | |
| | | ccggggaacttcaccctcaggggcctcccacaccttctcc | |
| | | agcgaggaggtctcagtcccagcctcgggagggcacctcc | |
| | | ttttctgtgctttcttccctgaggcattcttcctcatccc | |
| | | tagggtgttgtgtagaactctttttaaactctatgctccg | |
| | | agtagagttcatctttatattaaactteccctgttcaaat | |
| | | aa | |
| HMOX2 | NM_001127204.1 | catctctaggccccgccccgcgctgcgtgcccacgttgcg | 5 |
| | | ccggcctcgcgccagtccgctgggctgcagggactgcggc | |
| | | gcctgagggagtcgctgacgggcacgctgactggaggctg | |
| | | gcggacaggcgacagcgacctgcggcagagtcttgctgcg | |
| | | acacccaggctggagtgcaatggcgctatctcggctcact | |
| | | gcaacctccgcttcccggattcaagcgattctcctgcctc | |
| | | agcctcccgagtaggtgggactacaggaccagaggagcga | |
| | | gagcagcaagaaccacacccagcagcaatgtcagcggaag | |
| | | tggaaacctcagagggggtagacgagtcagaaaaaaagaa | |
| | | ctctggggccctagaaaaggagaaccaaatgagaatggct | |
| | | gacctctcggagctcctgaaggaagggaccaaggaagcac | |
| | | acgaccgggcagaaaacacccagtttgtcaaggacttctt | |
| | | gaaaggcaacattaagaaggagctgtttaagctggccacc | |
| | | acggcactttacttcacatactcagccctcgaggaggaaa | |
| | | tggagcgcaacaaggaccatccagcctttgcccctttgta | |
| | | cttccccatggagctgcaccggaaggaggcgctgaccaag | |
| | | gacatggagtatttctttggtgaaaactgggaggagcagg | |
| | | tgcagtgccccaaggctgcccagaagtacgtggagcggat | |
| | | ccactacatagggcagaacgagccggagctactggtggcc | |
| | | catgcatacacccgctacatgggggatctctcggggggcc | |
| | | aggtgctgaagaaggtggcccagcgagcactgaaactccc | |
| | | cagcacaggggaagggacccagttctacctgtttgagaat | |
| | | gtggacaatgcccagcagttcaagcagctctaccgggcca | |
| | | ggatgaacgccctggacctgaacatgaagaccaaagagag | |
| | | gatcgtggaggaggccaacaaggcttttgagtataacatg | |
| | | cagatattcaatgaactggaccaggccggctccacactgg | |
| | | ccagagagaccttggaggatgggttccctgtacacgatgg | |
| | | gaaaggagacatgcgtaaatgccctttctacgctgctgaa | |
| | | caagacaaaggtgccctggagggcagcagctgtcccttcc | |
| | | gaacagctatggctgtgctgaggaagcccagcctccagtt | |
| | | catcctggccgctggtgtggccctagctgctggactcttg | |
| | | gcctggtactacatgtgaagcacccatcatgccacaccgg | |
| | | taccctcctcccgactgaccactggcctacccctttctcc | |
| | | agccctgactaaactaccacctcaggtgactttttaaaaa | |
| | | atgctgggtttaagaaaggcaaccaataaaagccagatgc | |
| | | tagagcctctgcctgacagcatcctctctatgggccatat | |
| | | tccgcactgggcacaggccgtcaccctgggagcagtcggc | |
| | | acagtgcagcaagcctggcccccgacccagctctactcca | |
| | | ggcttccacacttctgggccctaggctgcttccggtagtc | |
| | | cctgtttttgcagtacatgggtgactatctcccctgttgg | |
| | | aggtgagtggcctgtaagtccaagctgtgcgagggggcct | |
| | | tgctggatgctgctgtacaacttctgggcctctcttggac | |
| | | cctgggagtgagggtgggtgtgggtggaagcctcagaggc | |
| | | cttgggagctcatccctctcacccagaatccctctaaccc | |
| | | cttgggtgcggtttgctcagccccagcttatctcctcctc | |
| | | cgcgctgtgtaaatgctccagcactcaataaagtgggctt | |
| | | tgcaagctaaaaaaaaaaaaaaaaaaaaaaaa | |
| MCM2 | NM_004526.3 | atgacgtcgcgttccgtagggctcttcccgggctttggtg | 6 |
| | | ggtcacgtgaaccacttttcgcgcgaaacctggttgttgc | |
| | | tgtagtggcggagaggatcgtggtactgctatggcggaat | |
| | | catcggaatccttcaccatggcatccagcccggcccagcg | |
| | | tcggcgaggcaatgatcctctcacctccagccctggccga | |
| | | agctcccggcgtactgatgccctcacctccagccctggcc | |
| | | gtgaccttccaccatttgaggatgagtccgaggggctcct | |
| | | aggcacagaggggcccctggaggaagaagaggatggagag | |
| | | gagctcattggagatggcatggaaagggactaccgcgcca | |
| | | tcccagagctggacgcctatgaggccgagggactggctct | |
| | | ggatgatgaggacgtagaggagctgacggccagtcagagg | |
| | | gaggcagcagagcgggccatgcggcagcgtgaccgggagg | |
| | | ctggccggggcctgggccgcatgcgccgtgggctcctgta | |
| | | tgacagcgatgaggaggacgaggagcgccctgcccgcaag | |
| | | cgccgccaggtggagcgggccacggaggacggcgaggagg | |
| | | acgaggagatgatcgagagcatcgagaacctggaggatct | |
| | | caaaggccactctgtgcgcgagtgggtgagcatggcgggc | |
| | | ccccggctggagatccaccaccgcttcaagaacttcctgc | |
| | | gcactcacgtcgacagccacggccacaacgtcttcaagga | |
| | | gcgcatcagcgacatgtgcaaagagaaccgtgagagcctg | |
| | | gtggtgaactatgaggacttggcagccagggagcacgtgc | |
| | | tggcctacttcctgcctgaggcaccggcggagctgctgca | |
| | | gatctttgatgaggctgccctggaggtggtactggccatg | |
| | | taccccaagtacgaccgcatcaccaaccacatccatgtcc | |
| | | gcatctcccacctgcctctggtggaggagctgcgctcgct | |
| | | gaggcagctgcatctgaaccagctgatccgcaccagtggg | |
| | | gtggtgaccagctgcactggcgtcctgccccagctcagca | |
| | | tggtcaagtacaactgcaacaagtgcaatttcgtcctggg | |
| | | tcctttctgccagtcccagaaccaggaggtgaaaccaggc | |
| | | tcctgtcctgagtgccagtcggccggcccctttgaggtca | |
| | | acatggaggagaccatctatcagaactaccagcgtatccg | |
| | | aatccaggagagtccaggcaaagtggcggctggccggctg | |
| | | ccccgctccaaggacgccattctcctcgcagatctggtgg | |
| | | acagctgcaagccaggagacgagatagagctgactggcat | |
| | | ctatcacaacaactatgatggctccctcaacactgccaat | |
| | | ggcttccctgtctttgccactgtcatcctagccaaccacg | |
| | | tggccaagaaggacaacaaggttgctgtaggggaactgac | |
| | | cgatgaagatgtgaagatgatcactagcctctccaaggat | |
| | | cagcagatcggagagaagatctttgccagcattgctcctt | |
| | | ccatctatggtcatgaagacatcaagagaggcctggctct | |
| | | ggccctgttcggaggggagcccaaaaacccaggtggcaag | |
| | | cacaaggtacgtggtgatatcaacgtgctcttgtgcggag | |
| | | accctggcacagcgaagtcgcagtttctcaagtatattga | |
| | | gaaagtgtccagccgagccatcttcaccactggccagggg | |
| | | gcgtcggctgtgggcctcacggcgtatgtccagcggcacc | |
| | | ctgtcagcagggagtggaccttggaggctggggccctggt | |
| | | tctggctgaccgaggagtgtgtctcattgatgaatttgac | |
| | | aagatgaatgaccaggacagaaccagcatccatgaggcca | |
| | | tggagcaacagagcatctccatctcgaaggctggcatcgt | |
| | | cacctccctgcaggctcgctgcacggtcattgctgccgcc | |
| | | aaccccataggagggcgctacgacccctcgctgactttct | |
| | | ctgagaacgtggacctcacagagcccatcatctcacgctt | |
| | | tgacatcctgtgtgtggtgagggacaccgtggacccagtc | |
| | | caggacgagatgctggcccgcttcgtggtgggcagccacg | |
| | | tcagacaccaccccagcaacaaggaggaggaggggctggc | |
| | | caatggcagcgctgctgagcccgccatgcccaacacgtat | |
| | | ggcgtggagcccctgccccaggaggtcctgaagaagtaca | |
| | | tcatctacgccaaggagagggtccacccgaagctcaacca | |
| | | gatggaccaggacaaggtggccaagatgtacagtgacctg | |
| | | aggaaagaatctatggcgacaggcagcatccccattacgg | |
| | | tgcggcacatcgagtccatgatccgcatggcggaggccca | |
| | | cgcgcgcatccatctgcgggactatgtgatcgaagacgac | |
| | | gtcaacatggccatccgcgtgatgctggagagcttcatag | |
| | | acacacagaagttcagcgtcatgcgcagcatgcgcaagac | |
| | | ttttgcccgctacctttcattccggcgtgacaacaatgag | |
| | | ctgttgctcttcatactgaagcagttagtggcagagcagg | |
| | | tgacatatcagcgcaaccgctttggggcccagcaggacac | |
| | | tattgaggtccctgagaaggacttggtggataaggctcgt | |
| | | cagatcaacatccacaacctctctgcattttatgacagtg | |
| | | agctcttcaggatgaacaagttcagccacgacctgaaaag | |
| | | gaaaatgatcctgcagcagttctgaggccctatgccatcc | |
| | | ataaggattccttgggattctggtttggggtggtcagtgc | |
| | | cctctgtgctttatggacacaaaaccagagcacttgatga | |
| | | actcggggtactagggtcagggcttatagcaggatgtctg | |
| | | gctgcacctggcatgactgtttgtttctccaagcctgctt | |
| | | tgtgcttctcacctttgggtgggatgccttgccagtgtgt | |
| | | cttacttggttgctgaacatcttgccacctccgagtgctt | |
| | | tgtctccactcagtaccttggatcagagctgctgagttca | |
| | | ggatgcctgcgtgtggtttaggtgttagccttcttacatg | |
| | | gatgtcaggagagctgctgccctcttggcgtgagttgcgt | |
| | | attcaggctgcttttgctgcctttggccagagagctggtt | |
| | | gaagatgtttgtaatcgttttcagtctcctgcaggtttct | |
| | | gtgcccctgtggtggaagagggcacgacagtgccagcgca | |
| | | gcgttctgggctcctcagtcgcaggggtgggatgtgagtc | |
| | | atgcggattatccactcgccacagttatcagctgccattg | |
| | | ctccctgtctgtttccccactctcttatttgtgcattcgg | |
| | | tttggtttctgtagttttaatttttaataaagttgaataa | |
| | | aatataaaaaaaaaaaaaaaaaaa | |
| PDXK | NM_003681.4 | cggaactcgcgggttcggagccgcccgctgaggtcagaag | 7 |
| | | gaggcgtctgcgctgatcgggtccgccgcgcgccagagcc | |
| | | agagtcgcagccgaggggagccggggccggagcccgagcc | |
| | | cgagccgagccggagcccgagcgagcggcggagaccgtgc | |
| | | ccccgcctcggccccgcgccgccgcggccaggcccggcat | |
| | | ggaggaggagtgccgggtgctctccatacagagccacgtc | |
| | | atccgcggctacgtgggcaaccgggcggccacgttcccgc | |
| | | tgcaggttttgggatttgagattgacgcggtgaactctgt | |
| | | ccagttttcaaaccacacaggctatgcccactggaagggc | |
| | | caagtgctgaattcagatgagctccaggagttgtacgaag | |
| | | gcctgaggctgaacaacatgaataaatatgactacgtgct | |
| | | cacaggttatacgagggacaagtcgttcctggccatggtg | |
| | | gtggacattgtgcaggagctgaagcagcagaaccccaggc | |
| | | tggtgtacgtgtgtgatccagtcttgggtgacaagtggga | |
| | | cggcgaaggctcgatgtacgtcccggaggacctccttccc | |
| | | gtctacaaagaaaaagtggtgccgcttgcagacattatca | |
| | | cgcccaaccagtttgaggccgagttactgagtggccggaa | |
| | | gatccacagccaggaggaagccttgcgggtgatggacatg | |
| | | ctgcactctatgggccccgacaccgtggtcatcaccagct | |
| | | ccgacctgccctccccgcagggcagcaactacctgattgt | |
| | | gctggggagtcagaggaggaggaatcccgctggctccgtg | |
| | | gtgatggaacgcatccggatggacattcgcaaagtggacg | |
| | | ccgtctttgtgggcactggggacctgtttgctgccatgct | |
| | | cctggcgtggacacacaagcaccccaataacctcaaggtg | |
| | | gcctgtgagaagaccgtgtctaccttgcaccacgttctgc | |
| | | agaggaccatccagtgtgcaaaagcccaggccggggaagg | |
| | | agtgaggcccagccccatgcagctggagctgcggatggtg | |
| | | cagagcaaaagggacatcgaggacccagagatcgtcgtcc | |
| | | aggccacggtgctgtgagggccccgccgcttgcccgtgac | |
| | | acgcagcgcgttggtgtctccgtgtttgtccctgtgaaaa | |
| | | catgtaacgtctgccttagagccatgaccgaaacttgata | |
| | | tttttttctttcatgagtgtccggcatctgctggtcttca | |
| | | ttgtgaaacgtgccagtcgtgctttgtgaaaaataacaaa | |
| | | gtggtcacagaaatttgtgatctgaaaacccggctccctt | |
| | | ccccacaaggctcctgggcctccgggaagacgggcccctg | |
| | | tttgccatctcgggggtgttccctgtgggagggtgagtgg | |
| | | gtgaggccgagcctgctgcgtgtggagcctcgagtgggcc | |
| | | ctggctgccactaccgtacagaggccgtgtcgcgctgggc | |
| | | tgggcctgggtggcctctgtctttgcatctctgagaagga | |
| | | gtcgggtggtaacggttggggtcaggaagaattctgccaa | |
| | | gtatctttactgtcattctgaccatagcctctttgttccc | |
| | | gcattcgaacttttggttcttactttgctgctcgtttagt | |
| | | ccctggggatttcagatcttaggctgttgtttcaccgtat | |
| | | gggagggttgatgtgagcttgcttggagacacacggtgca | |
| | | gcatcagggaccttcccaggccccagcaaattcaagtcgg | |
| | | tctgcagacctctcagctacccgcgggacctcttgtaacc | |
| | | catcggcatcttccaggaatccgccgagtgacttgaggaa | |
| | | gatgctaacgcagtaaggtctgtgctgggccaagagcagc | |
| | | tttgaagctccagagaaccaccccgtcaggttccttgtgg | |
| | | aagctcccctcatccgtggtgcagcaggctgagcactgcg | |
| | | cgtttgccacgtgctgcccgtgacagcacattgagccaca | |
| | | gcatttgtagacaggacagaggggtgcctgccccctgccc | |
| | | ctgctggcacatttaacccttgtcccctgacctcagttct | |
| | | gtgccccaccaaatgcccaggggcaagaggccaccctgga | |
| | | agctgccaatcttccaaggtgggtgtggggcacggtgggg | |
| | | gcgggcagctcccaggcccttgggcaggctggggtgacgg | |
| | | cagaggccacagcaccagctctgacaagtcctatcatcct | |
| | | ctgctcagcagtgacctccctggccccactttgcccagag | |
| | | tttggggtccccccaggtatagctataggcggcagtgcct | |
| | | gtccctggcctgccttgatttcagccacacccctgcagcc | |
| | | ctgcatcccagctctggggtgtgcagaggtttgtgtctcc | |
| | | agggaacccacggctggagagaaatagggagatgcaggaa | |
| | | gtgggggcccatggggcccccaagaagcggactctccaag | |
| | | gggtacccccaccccgctaccttccccacggacgggcccc | |
| | | tcctggagcccataccctcctgtgaggccattccagtgtc | |
| | | ttctagaaagactcgcttgccaggagtgcgttctttgttg | |
| | | aaaaatgccctgaagcgaaaagatgcaggtttatatggaa | |
| | | cccccaccccctcccccactctcccactctgttcgttctg | |
| | | aatgtcttcacgagcgtgcatcagggcgcctggctccccc | |
| | | acctcagccagtgagtcagacacgggtttcgcagccatgt | |
| | | ttcctggctccgaggacacgggtggcaggcccgttgcagc | |
| | | ccagagccactggtccctacagggcgccgccacaccagca | |
| | | ggaaggaggatggctgtgtccggagcctggcggggaggcg | |
| | | gcctccccagtatgtgagtgcagggatctgccagaaccac | |
| | | ctggccctctgtagggcgtttaactggaaataccctcact | |
| | | gccaagtggagactggggcgtgtgccacattgccagccac | |
| | | caggaaagcttttctttttcttttttttttttttttaaac | |
| | | accaagagcacgtatagcatgggggaaagaacctaaatgt | |
| | | ctctctgtcctgtgagctggtgaaaaacccagcatgagaa | |
| | | cgcagtgtcaggtgtgggactccttctgcccctgcagtgg | |
| | | gtgttacgggcggtgtgccctggcgagcaagctttgattc | |
| | | ttggttctttgagctcgtttcagaggctgagtccccacat | |
| | | cagctttagttcttggacttccctgtattaagcaagaatt | |
| | | aggagaatggctgtccctgcaggcgcctcccgtaaatcct | |
| | | gagctctctggcgcaatctgaaacttctcttctgttttct | |
| | | ttggctgtatcagccgaaccaggagaggcctgggctgcga | |
| | | ctaaggagaaagaaatcgggggtttctgagagcagatggt | |
| | | gcctttgtgggtgcagggcttttgtggaaattgtcagcct | |
| | | ctacgggcagagtccggcatcccctccccagactgcctgc | |
| | | tgtcaaaccacggagcagctggagcctgccctgtccacgg | |
| | | cccgtttccacccgggcatgttcgtctctcatgacttcgg | |
| | | cagaggcccctggtggccttcagtttcagtttctcatcca | |
| | | ggaaggtaaccttgggcattggcagtgggtttccctatgg | |
| | | cttggatccagattagaattgatctttgttttcactttcc | |
| | | atagttaataacatgcaaaataatgagaagaatttatttt | |
| | | aaggtgacagctatactggtccaacatcgcctgcttattg | |
| | | tcagggtacagaagtttaatactttcttaatccagttttt | |
| | | caaacttctccctgtagaccgtaaggatgaattccacaat | |
| | | aggatcctttttaaaatcgattttaaattgttgcctagtc | |
| | | ctgccaaggttattatgtgcatctgttatttttccaatac | |
| | | atgtaaacagttgcagcatgatgctttgtttaatgtcctg | |
| | | ttcttaagctcgttagagccagttttgaaacgtttggtct | |
| | | taccgtgaacggaggctggcttggcttagccacgctgatg | |
| | | agtaagtgagggatgtctccatcttgagatcaccaggcaa | |
| | | gagagttgcctgcaccaggtaagaggccaaagcccctggg | |
| | | gtaacagtccccaccgctacccgaggtaaaacaataaaag | |
| | | ctatgtggttgagctcaggcctctcgtgcctggtgtcaga | |
| | | gaaggcagagcccacagtaggtgcacggtgcaaggccctg | |
| | | ggagggcactggccagggaaggtggtatagatggccctca | |
| | | gattgcggggccccgagcagctccccactctgcccgtcca | |
| | | ccttccctggctccagcctcattctctctttagtttaact | |
| | | atgcaaagagaggaggttgagagtgttctggcagctggag | |
| | | ctcttttccttgtccttcctgccctccgatggggccacct | |
| | | gtgtcggggcagcagtgtccatgtttatggagatcagagg | |
| | | tgtccccactgtgtggctggactgtactctgctgcccggg | |
| | | tagccaggagtctctccctctctcccctgccgcctgcctg | |
| | | gtctcatgggcctccttcacacacccctccctgtggatcg | |
| | | cctgcctgggcccagagcaggggaactggagtttgtgagt | |
| | | gagcagagcaggttatgtgcagacagggaaacgagaactt | |
| | | tggacctggctttctgagtccaggtgagagctgtgtggcc | |
| | | ccccgatgccactctgcccgccggagggatgtgcctgctg | |
| | | agccttttccttccacgccgcctctcactgccaggccagc | |
| | | ggcttccgctgagactcgctggagaggcggctcccgtgtc | |
| | | cgtccaccgagcactcagatggatgctgatcaccagggcc | |
| | | gagggggctcccagaaggaccccaggccctggggagggtg | |
| | | gctgtgggaggccaagtccactgcccggaagtcttgtcag | |
| | | ccctaagccagggaagcctggagcgtggcctggcgggtct | |
| | | gggtggacaccgtccccactccggactcccagcacagggg | |
| | | aggatacctgagcctgtatggccctgtagccctgggcaga | |
| | | gctgggcctgtcgtgtgttcctgcctggcaggtgcaggtg | |
| | | ctggccatctgcaggtggaaggaggtgggaatcttggatt | |
| | | ttttgtttttttttgtttttttttttttgagatgaagtct | |
| | | cgctctgacacccaggctggcgtgcagtggtgtgatctcg | |
| | | gctcactgcaaactccgcttcctgggttcaagtggttctc | |
| | | ctgccccagcctcccaagtagctgggattacaggcatgcg | |
| | | ccaccacgctcagctgatttttgtatttttagtagagatg | |
| | | gggtttcaccatgttggccaagctggtctcaaactcctga | |
| | | cctcaagtgatctgcccgcctcggcctcccagagtgctgg | |
| | | gattacaggcatgagccagtgcacccggcggaatcttgga | |
| | | atttttatagacagcacctcagtttctgactccagccgca | |
| | | cacctcctgcctctgccagcaggggttgccgccagaccag | |
| | | agccagggccaggtccctgcgtccatcccccccggtagga | |
| | | tggacgtgagccatccttctaggggacttttttcagtgtg | |
| | | cgactcgtctctgttaggtggtaggagccagtttgtgtgg | |
| | | cctgtgccacgctccacagtgcgtggctgggctctgtgtg | |
| | | tggcctgtgtcccctgtccctgcaggacccagcaggcatc | |
| | | gtggcgtgacagctgtgtccaagccactgcccgggcatcc | |
| | | catcacccaccagggtgcacggtctctcctgctgggggct | |
| | | ttctgtcgcatgtgtgtctcctgtcgactctgcagtttgt | |
| | | tctcagagcagaatgtttcctgttctcagtgcacaaagac | |
| | | actggttttcaatcggcgtctaaaaccacgttcctgcctt | |
| | | tcattgcaacacggtgtgttcatttgtttaaaacagttta | |
| | | atgagtaagtttagatgactggtcaatatcttaaaaatgt | |
| | | atattagtaagaagttcttcctggaatttttctttcgatt | |
| | | ctggcagaataaacaggtgtttttagttttcccactgtct | |
| | | gagccaagcaggaccctgtcccagagcaagagatgtcccc | |
| | | ttccatctctgacccttgcctgggacaagctttgatgggg | |
| | | ggccccagcttcaaggctgtggtgggaacagcacccccaa | |
| | | atgccagcctctcctttcttcccatccaccagtatactgc | |
| | | ggggccatttctggtctttgtccaacaggaaacccatttc | |
| | | tggtgggatatgccttccagtgccacagggccactcaccc | |
| | | catgcatctctgtcctgcccgtcagtgctgggacggacag | |
| | | caagggcaagcccagtgtctggcggataggtgggtgggaa | |
| | | cagagaggggagaatgccgtcctaagcttctgcttgggga | |
| | | tcccccacacgacctgggtactgcctgggaaacctgtcct | |
| | | aagtaaaactatggacctcgcctcgcccaccggcctgcga | |
| | | agccagcatctccgtgaaggtggatggaagcgcctttgtc | |
| | | ctcattttgagctgcaagctgggtcagcggctctgaagcc | |
| | | ctcgagtgactttctaacccaagacccagcccctggcagg | |
| | | aggagggtgggtgcagggctggtgggacaaaaagaggcct | |
| | | cagcaggcctggaagacccttccagtacatcccacagcgt | |
| | | gtcgagcagctgggagaacctgtgtcaagctcgagccgtc | |
| | | ataggtccccatgaggtgtctgaagccccttcttggtgat | |
| | | gggaggcagaggtgctgacgttctggagcatggacgtgag | |
| | | tcctcagctggctccgcgtgggcccttggagggtgccagg | |
| | | tgtgtggtgaccttctggatgcctttaacttcatggctgc | |
| | | gtcattcctgatttagaactttaaccggagcttcatctag | |
| | | tgattgcaaaactggaccaatgggaggacggcggcgcagc | |
| | | ccgctccctccgtggaatggagctcagctcttcggaggca | |
| | | tcaaagcacctgtcgcctccgtggtccccctgctgaggga | |
| | | gtgcggcctctgcaaggttcgggggtggcttcgtttgcct | |
| | | ggagtggccggccctgcttgtgccatgtggatgtttgtga | |
| | | gcctcggtcctacagcactgtgtaggctgcatctgtttcg | |
| | | tgctggtcctgttgacttgtatgatatccacaaataaata | |
| | | ttttcatggcggtcgtgttgaaaaaaaaaa | |
| POP7 | NM_005837.2 | ggaaggggcggggcgaacggaagccgggaaggcgattcat | 8 |
| | | agctcgcggggtacgggcgcgcgtgcgcactccgcagccc | |
| | | gttcaggaccccggcgcgggcagggcgcccacgagctggc | |
| | | tggctgcttgcacccacatccttctttctctgggacctgg | |
| | | ggtcgcggttacttgggctggccggcgaacccttgagtgg | |
| | | cctggcggggagcgggcctcgcgcgcctggagggccctgt | |
| | | ggaacgaagagaggcacacagcatggcagaaaaccgagag | |
| | | ccccgcggtgctgtggaggctgaactggatccagtggaat | |
| | | acacccttaggaaaaggcttcccagccgcctgccccggag | |
| | | acccaatgacatttatgtcaacatgaagacggactttaag | |
| | | gcccagctggcccgctgccagaagctgctggacggagggg | |
| | | cccggggtcagaacgcgtgctctgagatctacattcacgg | |
| | | cttgggcctggccatcaaccgcgccatcaacatcgcgctg | |
| | | cagctgcaggcgggcagcttcgggtccttgcaggtggctg | |
| | | ccaatacctccaccgtggagcttgttgatgagctggagcc | |
| | | agagaccgacacacgggagccactgactcggatccgcaac | |
| | | aactcagccatccacatccgagtcttcagggtcacaccca | |
| | | agtaattgaaaagacactcctccacttatcccctccgtga | |
| | | tatggctcttcgcatgctgagtactggacctcggaccaga | |
| | | gccatgtaagaaaaggcctgttccctggaagcccaaagga | |
| | | ctctgcattgagggtgggggtaattgtctcttggtgggcc | |
| | | cagttagtgggccttcctgagtgtgtgtatgcggtctgta | |
| | | actattgccatataaataaaaaatcctgttgcactagtgt | |
| | | cctgccatcccaaaaaaaaaaaaaaaaaa | |
| S100P | NM_005980.2 | tgaggctgccttataaagcaccaagaggctgccagtggga | 9 |
| | | cattttctcggccctgccagcccccaggaggaaggtgggt | |
| | | ctgaatctagcaccatgacggaactagagacagccatggg | |
| | | catgatcatagacgtcttttcccgatattcgggcagcgag | |
| | | ggcagcacgcagaccctgaccaagggggagctcaaggtgc | |
| | | tgatggagaaggagctaccaggcttcctgcagagtggaaa | |
| | | agacaaggatgccgtggataaattgctcaaggacctggac | |
| | | gccaatggagatgcccaggtggacttcagtgagttcatcg | |
| | | tgttcgtggctgcaatcacgtctgcctgtcacaagtactt | |
| | | tgagaaggcaggactcaaatgatgccctggagatgtcaca | |
| | | gattcctggcagagccatggtcccaggcttcccaaaagtg | |
| | | tttgttggcaattattcccctaggctgagcctgctcatgt | |
| | | acctctgattaataaatgcttatgaaatga | |
| SNRPA | NM_004596.4 | ggcggggccaggagagaaagctttgtggtttggtctcagg | 10 |
| | | gaagtagcaggcgccggttgagagaactacggccctgtcg | |
| | | gaaggtaacctccggtgcaaacgaccatcggcggcaggcg | |
| | | agcggtacgcttggcgtccgggccttcctgggcccgtctg | |
| | | aggaaacttgctgctcgaggccaggctgcctaggacctgt | |
| | | cccttttttctatactggctcccacatccgggttttttct | |
| | | ccgggacggcccttcggatgcttgggccaatgggaatcgc | |
| | | catttagggtgctccgcccaccgggtcgcgtagagcatcc | |
| | | tggaagtcgtagtaaatctctcgagagttctctccgcacg | |
| | | cgggctggagaagcgggtcctacgcacgctttgttgtcgc | |
| | | gctttgcctccgtccttgcccctactcccgccttacctga | |
| | | cttccttttcggaggaagatccttgagcagccgacgttgg | |
| | | gacaaaggatttggagaaacccagggctaaagtcacgttt | |
| | | ttcctcctttaagacttacctcaacacttcactccatggc | |
| | | agttcccgagacccgccctaaccacactatttatatcaac | |
| | | aacctcaatgagaagatcaagaaggatgagctaaaaaagt | |
| | | ccctgtacgccatcttctcccagtttggccagatcctgga | |
| | | tatcctggtatcacggagcctgaagatgaggggccaggcc | |
| | | tttgtcatcttcaaggaggtcagcagcgccaccaacgccc | |
| | | tgcgctccatgcagggtttccctttctatgacaaacctat | |
| | | gcgtatccagtatgccaagaccgactcagatatcattgcc | |
| | | aagatgaaaggcaccttcgtggagcgggaccgcaagcggg | |
| | | agaagaggaagcccaagagccaggagaccccggccaccaa | |
| | | gaaggctgtgcaaggcgggggagccacccccgtggtgggg | |
| | | gctgtccaggggcctgtcccgggcatgccgccgatgactc | |
| | | aggcgccccgcattatgcaccacatgccgggccagccgcc | |
| | | ctacatgccgccccctggtatgatccccccgccaggcctt | |
| | | gcacctggccagatcccaccaggggccatgcccccgcagc | |
| | | agcttatgccaggacagatgccccctgcccagcctctttc | |
| | | tgagaatccaccgaatcacatcttgttcctcaccaacctg | |
| | | ccagaggagaccaacgagctcatgctgtccatgcttttca | |
| | | atcagttccctggcttcaaggaggtccgtctggtacccgg | |
| | | gcggcatgacatcgccttcgtggagtttgacaatgaggta | |
| | | caggcaggggcagctcgcgatgccctgcagggctttaaga | |
| | | tcacgcagaacaacgccatgaagatctcctttgccaagaa | |
| | | gtagcaccttttccccccatgcctgccccttcccctgttc | |
| | | tggggccacccctttcccccttggctcagccccctgaagg | |
| | | taagtccccccttgggggccttcttggagccgtgtgtgag | |
| | | tgagtggtcgccacacagcattgtacccagagtctgtccc | |
| | | cagacattgcacctggcgctgttaggccggaattaaagtg | |
| | | gctttttgaggtttggtttttcacaatcaaaaaaaaaaaa | |
| | | aaaaaa | |
| SORD | NM_003104.5 | ctccacgctagcgccgcccaggctggcacaaaggaggaag | 11 |
| | | cctagtcccgcccctgcgtgcggcgcttctcccaggcccc | |
| | | accttccatccagtgccctggaccctcggctgggtagcgc | |
| | | caccagagcgaccaaacgtcccgcgccttccaggccgcac | |
| | | tccagagccaaaagagctccatggcggcggcggccaagcc | |
| | | caacaacctttccctggtggtgcacggaccgggggacttg | |
| | | cgcctggagaactatcctatccctgaaccaggcccaaatg | |
| | | aggtcttgctgaggatgcattctgttggaatctgtggctc | |
| | | agatgtccactactgggagtatggtcgaattgggaatttt | |
| | | attgtgaaaaagcccatggtgctgggacatgaagcttcgg | |
| | | gaacagtcgaaaaagtgggatcatcggtaaagcacctaaa | |
| | | accaggtgatcgtgttgccatcgagcctggtgctccccga | |
| | | gaaaatgatgaattctgcaagatgggccgatacaatctgt | |
| | | caccttccatcttcttctgtgccacgccccccgatgacgg | |
| | | gaacctctgccggttctataagcacaatgcagccttttgt | |
| | | tacaagcttcctgacaatgtcacctttgaggaaggcgccc | |
| | | tgatcgagccactttctgtggggatccatgcctgcaggag | |
| | | aggcggagttaccctgggacacaaggtccttgtgtgtgga | |
| | | gctgggccaatcgggatggtcactttgctcgtggccaaag | |
| | | caatgggagcagctcaagtagtggtgactgatctgtctgc | |
| | | tacccgattgtccaaagccaaggagattggggctgattta | |
| | | gtcctccagatctccaaggagagccctcaggaaatcgcca | |
| | | ggaaagtagaaggtcagctggggtgcaagccggaagtcac | |
| | | catcgagtgcacgggggcagaggcctccatccaggcgggc | |
| | | atctacgccactcgctctggtgggaacctcgtgcttgtgg | |
| | | ggctgggctctgagatgaccaccgtacccctactgcatgc | |
| | | agccatccgggaggtggatatcaagggcgtgtttcgatac | |
| | | tgcaacacgtggccagtggcgatttcgatgcttgcgtcca | |
| | | agtctgtgaatgtaaaacccctcgtcacccataggtttcc | |
| | | tctggagaaagctctggaggcctttgaaacatttaaaaag | |
| | | ggattggggttgaaaatcatgctcaagtgtgaccccagtg | |
| | | accagaatccctgatgttaatgggctctgccctcatcccc | |
| | | acagtcttgggatctcagggcacaatggctggacatgggt | |
| | | gggctctgatgcagaactttctcttttgaatgttaagaat | |
| | | aactaatacaattcattgtgaacagaagtccttaagcaga | |
| | | ggaattggtgtgccttaaagatacaatctgggatagtttg | |
| | | ggggaacttgtagccagaatgccctgttcatgctgagcaa | |
| | | agttcagcaagtagagcagagtttggcaggcaggtgccag | |
| | | gaactccccttcttcctggagtgccttcattgaggaagga | |
| | | aatctggcccttgggtttcctggttccactgctactgacc | |
| | | cagaggggaatgagggctgagttatgaaaagataacttca | |
| | | tgaagacttaactggcccagaagctgattttcatgaaaat | |
| | | ctgccactcagggtctgggatgaaggcttgtcagcacttc | |
| | | cagtttagaacgcaatgtttctagagacatattggctgtt | |
| | | tgttttgatgataaaaggagaataagaaaaggcatcactt | |
| | | tcctggatccaggataatttttaaaccaatcaaatgaaaa | |
| | | aaacaaacaaacaaaaaaggaaatgtcatgtgaggttaaa | |
| | | ccagtttgcattcccctaatgtggaaaaagtaagaggact | |
| | | actcagcactgtttgaagattgcctcttctacagcttctg | |
| | | agaattgtgttatttcacttgccaagtgaaggaccccctc | |
| | | cccaacatgccccagcccacccctaagcatggtcccttgt | |
| | | caccaggcaaccaggaaactgctacttgtggacctcacca | |
| | | gagaccaggagggtttggttagctcacaggacttccccca | |
| | | ccccagaagattagcatcccatactagactcatactcaac | |
| | | tcaactaggctcatactcaattgatggttattagacaatt | |
| | | ccatttctttctggttattataaacagaaaatctttcctc | |
| | | ttctcattaccagtaaaggctcttggtatctttctgttgg | |
| | | aatgatttctatgaacttgtcttattttaatggtgggttt | |
| | | tttttctggtaagatttagacctaaatcgcatcatgccaa | |
| | | cttgtgactttgagactattcatcaagaatgaggatatag | |
| | | tagccatgacatagcttgagctatagcctttaattcctta | |
| | | ctttggctatgggtggagggtgagtttgaagaggttctga | |
| | | ttttcttgtaacctgggaaagccatgaccttgtgcccgat | |
| | | tctttcagattgctttgggtaataaatattggtggtggta | |
| | | tctgactcatgctgctgtttatggtcctgtttagtgggga | |
| | | atggactcaggttacccatttcccagagggaaggatccca | |
| | | ggatttttgaaggttacatattttctgtaccaaatataat | |
| | | ttcattgacatgaattatctctaatcctcatgacaagcca | |
| | | catacacaatcattttgtagataaagaagatataaatgcc | |
| | | agaggagaccttaagattgtcttacaacacaacccttcag | |
| | | ttaacgagagagg | |
| STOML2 | NM_001287031.1 | tccgggggagcggaactgcaagaggaaaggctcgggtagg | 12 |
| | | cttctgggagcgaccgctccgctcgtctcgttggttccgg | |
| | | aggtcgctgcggcggtgggaaatgctggcgcgcgcggcgc | |
| | | ggggcactggggcccttttgctgaggggctctctactggc | |
| | | ttctggccgcgctccgcgccgcgcctcctctggattgccc | |
| | | cgaaacaccgtggtactgttcgtgccgcagcaggaggcct | |
| | | gggtggtggagcgaatgggccgattccaccggatcctgga | |
| | | gcctggtttgaacatcctcatccctgtgttagaccggatc | |
| | | cgatatgtgcagagtctcaaggaaattgtcatcaacgtgc | |
| | | ctgagcagtcggctgtgactctcgacaatgtaactctgca | |
| | | aatcgatggagtcctttacctgcgcatcatggacccttac | |
| | | aaggcaagctacggtgtggaggaccctgagtatgccgtca | |
| | | cccagctagctcaaacaaccatgagatcagagctcggcaa | |
| | | actctctctggacaaagtcttccgggtggaggcagagcgg | |
| | | cggaaacgggccacagttctagagtctgaggggacccgag | |
| | | agtcggccatcaatgtggcagaagggaagaaacaggccca | |
| | | gatcctggcctccgaagcagaaaaggctgaacagataaat | |
| | | caggcagcaggagaggccagtgcagttctggcgaaggcca | |
| | | aggctaaagctgaagctattcgaatcctggctgcagctct | |
| | | gacacaacataatggagatgcagcagcttcactgactgtg | |
| | | gccgagcagtatgtcagcgcgttctccaaactggccaagg | |
| | | actccaacactatcctactgccctccaaccctggcgatgt | |
| | | caccagcatggtggctcaggccatgggtgtatatggagcc | |
| | | ctcaccaaagccccagtgccagggactccagactcactct | |
| | | ccagtgggagcagcagagatgtccagggtacagatgcaag | |
| | | tcttgatgaggaacttgatcgagtcaagatgagttagtgg | |
| | | agctgggcttggccagggagtctgggaacaaggaagcaga | |
| | | ttttcctgattctggctctagcttccctgccaagattttg | |
| | | gtttttatttttttatttgaactttagtcgtgtaataaac | |
| | | tcaccagtggcaaaccagaaactgtcctctttgattgggg | |
| | | aatgaagttgggaaagtcactagcattttccttggatcca | |
| | | gtcctgtcagcatgatgcctccatgaataagagtgaactt | |
| | | cttgtaaagtgaaact | |
| UMPS | NM_000373.3 | ctgcagacgaggcaggcggaagaggcgggacttcgcgggt | 13 |
| | | gacgtcatcggggcgccggaggcccggggcgcctgggaat | |
| | | ttgaagcaaacaggcagcgcgcgacaatggcggtcgctcg | |
| | | tgcagctttggggccattggtgacgggtctgtacgacgtg | |
| | | caggctttcaagtttggggacttcgtgctgaagagcgggc | |
| | | tttcctcccccatctacatcgatctgcggggcatcgtgtc | |
| | | tcgaccgcgtcttctgagtcaggttgcagatattttattc | |
| | | caaactgcccaaaatgcaggcatcagttttgacaccgtgt | |
| | | gtggagtgccttatacagctttgccattggctacagttat | |
| | | ctgttcaaccaatcaaattccaatgcttattagaaggaaa | |
| | | gaaacaaaggattatggaactaagcgtcttgtagaaggaa | |
| | | ctattaatccaggagaaacctgtttaatcattgaagatgt | |
| | | tgtcaccagtggatctagtgttttggaaactgttgaggtt | |
| | | cttcagaaggagggcttgaaggtcactgatgccatagtgc | |
| | | tgttggacagagagcagggaggcaaggacaagttgcaggc | |
| | | gcacgggatccgcctccactcagtgtgtacattgtccaaa | |
| | | atgctggagattctcgagcagcagaaaaaagttgatgctg | |
| | | agacagttgggagagtgaagaggtttattcaggagaatgt | |
| | | ctttgtggcagcgaatcataatggttctcccctttctata | |
| | | aaggaagcacccaaagaactcagcttcggtgcacgtgcag | |
| | | agctgcccaggatccacccagttgcatcgaagcttctcag | |
| | | gcttatgcaaaagaaggagaccaatctgtgtctatctgct | |
| | | gatgtttcactggccagagagctgttgcagctagcagatg | |
| | | ctttaggacctagtatctgcatgctgaagactcatgtaga | |
| | | tattttgaatgattttactctggatgtgatgaaggagttg | |
| | | ataactctggcaaaatgccatgagttcttgatatttgaag | |
| | | accggaagtttgcagatataggaaacacagtgaaaaagca | |
| | | gtatgaaggaggtatctttaaaatagcttcctgggcagat | |
| | | ctagtaaatgctcacgtggtgccaggctcaggagttgtga | |
| | | aaggcctgcaagaagtgggcctgcctttgcatcgggggtg | |
| | | cctccttattgcggaaatgagctccaccggctccctggcc | |
| | | actggggactacactagagcagcggttagaatggctgagg | |
| | | agcactctgaatttgttgttggttttatttctggctcccg | |
| | | agtaagcatgaaaccagaatttcttcacttgactccagga | |
| | | gttcagttggaagcaggaggagataatcttggccaacagt | |
| | | acaatagcccacaagaagttattggcaaacgaggttccga | |
| | | tatcatcattgtaggtcgtggcataatctcagcagctgat | |
| | | cgtctggaagcagcagagatgtacagaaaagctgcttggg | |
| | | aagcgtatttgagtagacttggtgtttgagtgcttcagat | |
| | | acatttttcagatacaatgtgaagacattgaagatatgtg | |
| | | gtcctcctgaaagtcactggctggaaataatccaattatt | |
| | | cctgcttggattcttccacagggcctgtgtaagaatgggt | |
| | | tctggagttctcatggtctttaggaaatattgagtaattt | |
| | | gtaatcaccgcattgatactataataagttcattcttaag | |
| | | cttgctttttttgagactggtgtttgttagacagccacag | |
| | | tcctgtctgggttagggtcttccacatttgaggatccttc | |
| | | ctatctctccatgggactagactgctttgttattctattt | |
| | | attttttaatttttttcgagacaggatctcactctgttgc | |
| | | ccaggatggagtgcagtggtgagatcacggctcattgcag | |
| | | cctcgacctcccaggtgatcctcccacctcagcttccaga | |
| | | ttagctggtgctataggcatgcaccaccacgtccatctaa | |
| | | atttctttattatttgtagagatgaggtcttgccatgtta | |
| | | cccaggctggtctcaactcctgggctcaagcgatcctcct | |
| | | gcctcagtctctcaaagtgctgggattacaggtgtgagcc | |
| | | actgtgcccagcctaattgcagtaagacaaaaattctagg | |
| | | gcaccaagaggctaaagtcagcacagcttttcttgtgtcc | |
| | | tgtattctctgtctaatgtgttgcccaaataatacctaat | |
| | | tgttagccattcccctccatctctggcctaaaagtgatag | |
| | | tccaggtatccacatgggctggttcccagaactgccattg | |
| | | ctcactctccaaagaggggaaggtggggaaggggaaggtg | |
| | | actatagctcagctcctgagctagtatctggctgttattt | |
| | | caacaaccggagttggggtttgggctcattttttccccta | |
| | | gccagcaattatggaccagtagtaacacaagtgacagctt | |
| | | cctgtgactgacttcacaattaggaggtctaagattccat | |
| | | ttgggtatttgcttaaggatcccacataattgtcccaacg | |
| | | gtcattagtagaggggaggtaagccttcattaataataaa | |
| | | gagaaagcccacattcaaggtggtgtttgagcaggggcag | |
| | | ggtgagggctgtcccggtgctcattgcaccagcacactca | |
| | | cattccttctcatttggggcccacctgcaggaagtggcac | |
| | | aggatcagccatttccccacccttgtcagctgatggccca | |
| | | ctgttctttaatgactcagaggaatgcctaggattttttt | |
| | | ttttttttgagacagaatctcactgtcgcccaggctggag | |
| | | ttcagtggcacgatctcggctcactgcaacttctgcctct | |
| | | ctggttcaagcagttctcctgcctcagcctcccgagtagc | |
| | | tgggactacaagcctaggatttttaactcaggtttttatt | |
| | | atattccctcctgaagtttttacttcaagagcttctgctc | |
| | | taaagtccaatttgggcttcatgtccccagtgctgcatct | |
| | | ccagggaaatgctgtctgtgggagagaccaactctcaagg | |
| | | aagaagtggccacagaaggagcaggaagggagttggccct | |
| | | cagggctactctggggaagccaaaagtcatgaaggggaga | |
| | | agaattttctgacaaaaacttgcaggaatctcttaggtgt | |
| | | cttcagtgttggagtgatatgttgagaggcctttggagtg | |
| | | atgtgctgaggtctcaggcgcccacctccctggctgtcac | |
| | | ttccatgtgtcagtggttctcccactttagcaggtatcag | |
| | | agtcacctggagtcttgtcaaaacaggtaccagccccacc | |
| | | cgcagcgtttctgactctgggtagctctgggatggggctt | |
| | | gagaatttgcgtttccaaaaaggtcccaggtgatgctgcg | |
| | | gttgcctgcgcagggactggactttgagaaccacttcact | |
| | | ggttattcacatttctgcctctgcagtgagacagccttga | |
| | | ggtctgcctcctgctaagagtcacatgctcctgtccttta | |
| | | gaaatgtgggctcctgccatctccaggacgcaggcactgt | |
| | | tcctgttgatgaaccctatttcacaggacccctgctaagg | |
| | | tgatttgaggggaaatgagaggaggctcaaataatcaccc | |
| | | agcccctgccacttactgaaagtgtaggtccttgtgcccc | |
| | | acaccatcagagtttctgcgttagcagatttgtggtttgc | |
| | | ccagcagcctgggcgtgtgcatttctaatgggtgcctcaa | |
| | | gtgatctgtttctgatttgtatttctattgtgaagagtca | |
| | | gcccagtactgcaggcctcttacctaagcagaatcccagt | |
| | | ctggcatcaaagctttagaggacaagttgattcaggcaga | |
| | | gaagaacttgggctatacaagcgctgttcttcagcattga | |
| | | agtattttggaggcattagatagtttaaccctttctcagt | |
| | | caaggaatatttacagaacatgatctctgggcattgtaac | |
| | | tcctggtcttagtggggaatatagggaccccatgtctcca | |
| | | tggggtgcacagaatgtctgtgagactgatggagtggaga | |
| | | acgccatcccccagcctctccagctactcgaggcattctg | |
| | | tagaacataagcccatagattgtgtgtgtgtgtgtgtgtg | |
| | | tgtgtgtgtgtgtgtgtgtgtgcatgcgcgcgcgtgcgca | |
| | | ctggaggaacctaagaaactatttggtgcacttcctctta | |
| | | ttttagagctcccaaagtgtagctccagaatcgtaaaggg | |
| | | atatgctcagtctcacagccagcctgtggatctcagtccc | |
| | | aacactcacccttgtgctactgagtcagctctaagaaaat | |
| | | ctgccaaaagtaggccgagggctggttttttgttttgttt | |
| | | tgtttgtttgatacagggtcttcactctgttgcccaggct | |
| | | ggagtatatcatggctcactgcaaccttgacttgggctca | |
| | | agcgatccgctcaagtagctggaactactctcaagtagct | |
| | | ctcaagagcctctcgagtggctggaactacaggcgtgcac | |
| | | caccacagctggttaatttttaaaattttttgtagagacg | |
| | | gtggaggaggttctcactgtgactcagtgtgtgcccgaca | |
| | | gcagagcccacaccactccagttgcagtggttgccatctg | |
| | | ggtcatcagacctggctgtcaggggtgcagccacaggaga | |
| | | gccaacagcagagggtgctggccgctgagctagctgctaa | |
| | | tgctggcctgggtgcagttctcatccaaagtacccggtgg | |
| | | gtgggagtcactcagtaccagttccgagcctgaacccaaa | |
| | | ctctcgtgtttctgctcacccctctctggcttctgccacc | |
| | | acatgggaagaatatgccctggttagcccatggcttctga | |
| | | agagcaagagaaagtagagcagagcctactccagcctccc | |
| | | ccgtccaatgtatgaaagccccagctgatctgtaagcctg | |
| | | ggagcgtgataaatgcttagtagtgcatgccatggagttc | |
| | | cagggtggtttattacacggcaatatctagctaaatacat | |
| | | ttaacttgctgcagctctctggatccagcctggttaccag | |
| | | gaagacaaaaactgggctccaccaggaaccagtcttctgc | |
| | | cttcccaaccatcacctctggctgcatcagcgatctctcc | |
| | | cagcgaaatagctgcttggtcttgtgtgaatcctgtactt | |
| | | taacacagtggaccaagtgtcagtcattgaaaatgaccat | |
| | | gagtaaccctgtggactctctgcagcttggttcctttgcc | |
| | | ccttaacaggtgggtatgaatcgtgtcttcagtgccaggg | |
| | | ctgaatgagaaagggcattcctttttgaaggaatctgata | |
| | | ctaaacacaaagcatgagaaaaatcaggacttgttggagt | |
| | | tatatttttaaaatatatattttaacagttatatatatta | |
| | | gatataatatataatagtatatataaataatactatattg | |
| | | cccaggctggtctcgaactccttagctcaagtgatcctcc | |
| | | tgccttggcttcccaaagtgctaggattacaggtgtgagc | |
| | | cactgctcccggcctgttggagttctttacatttatttta | |
| | | taatcaatgctgttttattaaatgcggattttattttgga | |
| | | ttacaggatgtagaatgccatatttttcttagatcatagg | |
| | | gcctttcacatttgtaatttggccttgtatgagttaccct | |
| | | gcaatccctttgttttccccataacccttccaaaggaagg | |
| | | ccgcaatagaaatacaaagagaaacaaaataattagaata | |
| | | ttttttaacttctaaagttcaaggttttggcataagtctg | |
| | | gtttagaagcacatttgcctagccctttccttcccaccaa | |
| | | gggggaaagtcttcctctagacaagaggcagagggctcct | |
| | | cagagtcagatcctggtgtgggctctcacgtgctgctgct | |
| | | gaatcccagggaaggagggaggaagggcagttgacaccca | |
| | | aaataagggtggggaactgtcagcagaggaggtctgtgtc | |
| | | atgtttttcagcgctggggttggggggagcccaggagagc | |
| | | aggaagatccagagatccctcgccccagctcggccatgtg | |
| | | tgtctgggacagagcctgaggtggcctgagcttcctgtgg | |
| | | ctccagagtaacattatagagaagctgaattctcctgttt | |
| | | ttctgaaaagggcatgggagttagctgagaagcagacctg | |
| | | gtgggcctgagagtctcaatcgtcaggtaaggacagtcag | |
| | | tgggaagtggacgggccgcacaaccaaggttctcatgagg | |
| | | acaaccatgtcttcgggggtgcccttgtgcacagacagct | |
| | | ccatagtcctgcctccaatgtcccaacactgcattgtctc | |
| | | cctgcacttagcagccctgcagggtgagacttggggagga | |
| | | tcctgaaatgattgtatttaacaagacatgctgtccttgt | |
| | | ttacctggaacctagcaatgttgttttctgccacaacttg | |
| | | aatagatacttgaagcagagatgatgttgagttaaaaaaa | |
| | | atatatacataaaaatatgggttcttttcaacctgaatag | |
| | | atggcctaaaaattcaaa | |
| MORF4L1 | NM_001265603.1 | cggcgtgccctggggcggcgcgggcgcaggggcgcgtgcg | 14 |
| | | cggcgggctgtcgttggctggagcagcggctgcgcgggtc | |
| | | gcggtgctgtgaggtctgcgggcgctggcaaatccggccc | |
| | | aggatgtagagctggcagtgcctgacggcgcgtctgacgc | |
| | | ggagttgggtggggtagagagtagggggcggtagtcgggg | |
| | | gtggtgggagaaggaggaggcggcgaatcacttataaatg | |
| | | gcgccgaagcaggacccgaagcctaaattccaggaggttg | |
| | | ggatgaatgggttccggagagcagagtactcaaatacgtg | |
| | | gacaccaatttgcagaaacagcgagaacttcaaaaagcca | |
| | | atcaggagcagtatgcagaggggaagatgagaggggctgc | |
| | | cccaggaaagaagacatctggtctgcaacagaaaaatgtt | |
| | | gaagtgaaaacgaaaaagaacaaacagaaaacacctggaa | |
| | | atggagatggtggcagtaccagtgagacccctcagcctcc | |
| | | tcggaagaaaagggcccgggtagatcctactgttgaaaat | |
| | | gaggaaacattcatgaacagagttgaagttaaagtaaaga | |
| | | ttcctgaagagctaaaaccgtggcttgttgatgactggga | |
| | | cttaattaccaggcaaaaacagctcttttatcttcctgcc | |
| | | aagaagaatgtggattccattcttgaggattatgcaaatt | |
| | | acaagaaatctcgtggaaacacagataataaggagtatgc | |
| | | ggttaatgaagttgtggcagggataaaagaatacttcaac | |
| | | gtaatgttgggtacccagctactctataaatttgagagac | |
| | | cacagtatgctgaaattcttgcagatcatcccgatgcacc | |
| | | catgtcccaggtgtatggagcgccacatctcctgagatta | |
| | | tttgtacgaattggagcaatgttggcttatacacctctgg | |
| | | atgagaagagccttgctttattactcaattatcttcacga | |
| | | tttcctaaagtacctggcaaagaattctgcaactttgttc | |
| | | agtgccagcgattatgaagtggctcctcctgagtaccatc | |
| | | ggaaagctgtgtgagaggcactctcactcacttatgtttg | |
| | | gatctccgtaaacacatttttgttcttagtctatctcttg | |
| | | tacaaacgatgtgctttgaagatgttagtgtataacaatt | |
| | | gatgtttgttttctgtttgattttaaacagagaaaaaata | |
| | | aaagggggtaatagctccttttttcttctttctttttttt | |
| | | tttcatttcaaaattgctgccagtgttttcaatgatggac | |
| | | aacagagggatatgctgtagagtgttttattgcctagttg | |
| | | acaaagctgcttttgaatgctggtggttctattcctttga | |
| | | cactacgcacttttataatacatgttaatgctatatgaca | |
| | | aaatgctctgattcctagtgccaaaggttcaattcagtgt | |
| | | atataactgaacacactcatccatttgtgcttttgttttt | |
| | | ttttatggtgcttaaagtaaagagcccatcctttgcaagt | |
| | | catccatgttgttacttaggcattttatcttggctcaaat | |
| | | tgttgaagaatggtggcttgtttcatggtttttgtatttg | |
| | | tgtctaatgcacgttttaacatgatagacgcaatgcattg | |
| | | tgtagctagttttctggaaaagtcaatcttttaggaattg | |
| | | tttttcagatcttcaataaattttttctttaaatttcaaa | |
| | | gaacaaaaaaaaaaaaaaa | |
| MRPL19 | NM_014763.3 | gtagtcttgacgtgagctagctggcatggcggcctgcatt | 15 |
| | | gcagcggggcactgggctgcaatgggcctaggccggagtt | |
| | | tccaagccgccaggactctgctccccccgccggcctctat | |
| | | cgcctgcagggtccacgcggggcctgtccggcagcagagc | |
| | | actgggccttccgagcccggtgcgttccaaccgccgccga | |
| | | aaccggtcatcgtggacaagcaccgccccgtggaaccgga | |
| | | acgcaggttcttgagtcctgaattcattcctcgaagggga | |
| | | agaacagatcctctgaaatttcaaatagaaagaaaagata | |
| | | tgttagaaaggagaaaagtactccacattccagagttcta | |
| | | tgttggaagtattcttcgtgttactacagctgacccatat | |
| | | gccagtggaaaaatcagccagtttctggggatttgcattc | |
| | | agagatcaggaagaggacttggagctactttcatccttag | |
| | | gaatgttatcgaaggacaaggtgtcgagatttgctttgaa | |
| | | ctttataatcctcgggtccaggagattcaggtggtcaaat | |
| | | tagagaaacggctggatgatagcttgctatacttacgaga | |
| | | tgcccttcctgaatatagcacttttgatgtgaatatgaag | |
| | | ccagtagtacaagagcctaaccaaaaagttcctgttaatg | |
| | | agctgaaagtaaaaatgaagcctaagccctggtctaaacg | |
| | | ctgggaacgtccaaattttaatattaaaggaatcagattt | |
| | | gatctttgtttaactgaacagcaaatgaaagaagctcaga | |
| | | agtggaatcagccatggcttgaatttgatatgatgaggga | |
| | | atatgatacttcaaaaattgaagctgcaatatggaaggaa | |
| | | attgaagcgtcgaaaaggtcttgattctgagaatgaattt | |
| | | ggttagttgcagaagatacattggctctaagaggatatat | |
| | | tttgagaccaatttaatttcatttataagaacatagtaat | |
| | | taagtgaactaagcattcattgttttattaatactttttt | |
| | | tctaaaataaaacttgtacaccagtttattactctaaaaa | |
| | | gagaattacacatgccaaatggaccaatgtccatttgctt | |
| | | attggaggcaaagctacaatagaagtcagagcatcaccag | |
| | | aatggtctttaatgagcatggaacctgagcaaagggaata | |
| | | ggtgggatgaattttttttttaattgtgaaacaattcata | |
| | | agcacaatatgatttacagaataataaacattcatgtacc | |
| | | cactatcaggttaagaaatagaacatttattaatatgtag | |
| | | gaatgttaagaaataaaacatttaataagatctcagaaga | |
| | | ctccagtaaatctgcaattgtatctctctcctttttaaat | |
| | | gtaaatatcatcttgacttgttaattattcccttgcattt | |
| | | cttttagtttactgccaacacatatattcttcaacaatat | |
| | | atttaattttgaaaaacctgaaaaaaaaaacctgttagca | |
| | | agtataaaggggcagtattactattattgcatgaaggctt | |
| | | caagggaaacgttacagtctttgggtcatagtctggcttc | |
| | | agcttcctctgagagtttacagaggccaattttgagcaaa | |
| | | ttcatggctaaggttatgagtgagttctgctaaacagaag | |
| | | gctcaccacaaggtatctggcaggattatactgggtagct | |
| | | ggatgttgcagaaatgtggttagaggaagtaaactgtttt | |
| | | ttgatgctcacagcatgatgaatcaaactctgtatcttag | |
| | | gattaggttaaaacaatacctttggtatgatatgagtgtt | |
| | | gttgctgatccatgcagcatggattggaaagctggggtat | |
| | | aagcacacatgctaaagaaaaacatgtaatttggtccata | |
| | | ctcacctggatatactgttcctcaggttaaaaaatacagt | |
| | | actatcctaaatcttgaaggcaactctcagcctatccatt | |
| | | gagttaccttcagatctgccctctggttcctagctgtctt | |
| | | gggactaacttctttcctgcgctcagctgttttctggatt | |
| | | ccatgttttccattttattgagtactaacttgttttgctg | |
| | | cagcacatcctttggtagcttctagaggaagtttgtgtgg | |
| | | aggtaaaatttttgagaccttgcatgtctcatgtttgatt | |
| | | gatactttatacgtttaggtaggaggtaattttccttcag | |
| | | gactttaaaaatattgttgctccattttctttgtttctat | |
| | | tgttgtattgagaaatccaatgccattttgatttccccat | |
| | | cataaatttcatgatgatgtgtcttggtgtgggtctatat | |
| | | ttatccattgtattgggttttaggtgaacccttccagata | |
| | | gtaactcatttctgtcagttctgggaaacacttagcattg | |
| | | gttgatgatttattctctgctgctttgttctcccaactat | |
| | | tatttggatgttggatatccagcactgggtatctattttc | |
| | | ttacctccctcccttgaccccagtctctgttttttagctc | |
| | | tttagctcaatcttccaactctttgctattgtattttaaa | |
| | | atcttaagaccccttcttgatttgtagaagttccttttct | |
| | | tacaaccaaaaagcctttatctatggatttgttcacagat | |
| | | aaggggtattcaatatagtgtatttttttttcatttaaaa | |
| | | ttgtttgcgcatctatttcctccaaatttctttctgtatt | |
| | | tattttttgttgtctatatttcagacttttccaggatatc | |
| | | tgataatctttggctgtcttcttatggttgaaagagggac | |
| | | taaaaagcttggaaagcctttgggttgtgggaaggggctg | |
| | | tctttaggattatctgaatgggcttttttgggagtcccct | |
| | | cctccacatgaatattttggttttgtcagattccctagaa | |
| | | tagaggcttccaatctccttcctggaggggtctgtccagg | |
| | | aaggagattgtctaggggtctgtcagacagcagctttcag | |
| | | ctacttccttgatctttttcactaatgattatatagtcat | |
| | | ctaactactgtcaacaagtaatagatatcctatccttcac | |
| | | ttgtttagattatttgctgagataacctctcaaaagaacc | |
| | | tctcaaaataaaaggttaacaagagcctatatcttatatt | |
| | | tttcttctctttatcttgttagaagatagctattaaaacc | |
| | | tgttctttttctgtcttgataaacacacttcaatcttggt | |
| | | agaatggtagatgggacagtatattttaggacctaaagct | |
| | | ctgcaaatgtatgatcagcttgtaagtacaggtgctcaaa | |
| | | aacatgtaaacaatcatgctttttactctgtaggaatatc | |
| | | tttaaaattcttgtgaatttttccccagaagtaaagcaaa | |
| | | tcttcccccagaaataaaattaaatgtgcataatctaaag | |
| | | ctttttttttttattgtggtaggatatatatataaaacat | |
| | | aatttgccattgtaaacattttaaatttacaagtcagagg | |
| | | cattaattacatcacaatgttgtgaaattattactactat | |
| | | ttccaaaattttctcatcaccccaaactgaaactctgtaa | |
| | | ctgttgagcaataacctcattcctgtatctctcccaaccc | |
| | | caggtaacctcaaatctttctttttatctttgagacaagg | |
| | | tctcattctatcactcaggtaggagtgcagtggtgtgatc | |
| | | atagctcattgcagcctcaaaatcctgggctcaagcaatc | |
| | | ctccttgagtagctaagactataggcacacattaactgcg | |
| | | cctggctgattttgttttttgtagagatgtggtcttgcta | |
| | | tgtttcccatgctggtcttgagttcctggcctcaagcagt | |
| | | ccttaagattcatccatgttgtggcatgtgtcagaatttc | |
| | | atttgtttttatgactaaataatattccattgtatgtata | |
| | | tacattttgttcatccatcttctgatgaacactgggatat | |
| | | gtctaccttttggctattgtgaataatgctgcagtaaaca | |
| | | ttgacataacaagtatgtatttgattgcctgtttctaagt | |
| | | tcttttgggtatacatcttgagtagaattgctagataatg | |
| | | tcatgttttatttctcttgtgatttcttcttcgatcccct | |
| | | ggttgagtgtgttaatttctacatgtttatgaatttccca | |
| | | ctgtttttttgttattgatttccaagttcattccattgtg | |
| | | attagagaagatacttagtatgattttaatgtttttgaga | |
| | | attggtgtgtggcctgatagatggtctgtcctggagaatg | |
| | | ttcctcatacacttgagcaaaatatttatcatgctattgt | |
| | | tgactgtagttttctatatgtctcttaggtcaaggtggtt | |
| | | tacaatgtgttaaggttctctttttttaaaaaaatttttg | |
| | | cacagagtatctttttctatgtgttccatgtatttgtgtc | |
| | | tttggagctatagtctcttgtagacagcatatcactatct | |
| | | tgttttgttttgttttttctgtccattctgccaatttctg | |
| | | ccttttgattggaaaatttaatccatttgcatttaaagta | |
| | | attaaggaaggactttcttctaccatttaacacttcttct | |
| | | atatgtcatatacttttttggcccctcatttcctctttat | |
| | | ggccttcttttctgtttttttgtagtgaactagtctgatt | |
| | | ctctttccactcccctttgtgtatatttgttagatgtttt | |
| | | atttgtggttgctatggggattatagttaacatcctacac | |
| | | ttaaaacaatctaatttaaactgataccaatttaccttca | |
| | | atagcatacaaaatctctactcctgtaaagctctgcccct | |
| | | gccccccttatgttattgatggcacaaattgcctaataaa | |
| | | taatttatagttatttgtatgagtttgtcttttaaatcat | |
| | | ttaggaaataaaaagtggagttagaaaacagtatgatagt | |
| | | aatactgacttttatatttgtcaatatatttatcttattt | |
| | | tggatccttatttcattatatagatttgagttactgtcta | |
| | | gtgcccttccatttcggcccaaaggattcccttatgcatt | |
| | | tcttgcagggcaagtctaattgtaataaactccctcagct | |
| | | tttgttttatctgagaatgtcttgatttctcccttatttt | |
| | | tgatggataattttgccagatacatgaatttttggtaaca | |
| | | gtatttttctttcagcactttaaatatgtcatcccactac | |
| | | cttctgacttcatggtttctcatgagatattagatgttat | |
| | | aaaatttgaggattcctcattcttgatgagtcagttctgt | |
| | | cttattgcttttcggatttgctcagcttttgtcttttgac | |
| | | agtttgattataacgcggctcagtgtgggtctctgagttt | |
| | | atcccacttagagtttgttgagtttcttggagtcatagat | |
| | | ttatgtcttttatcaaattttggacatatttggctattat | |
| | | ttcttcaatttttttcactgcttctttcttttccttctga | |
| | | aatattcttaatgtatatgttggtctgtttgatgctgtct | |
| | | caccagtttcttaggctgtgttctcttttgttcctcagac | |
| | | ttgattattgcagttgcccttctttttatttttttcaagt | |
| | | ttgttgattcttctccctgttcagatcaactgttgaactc | |
| | | ctctagtgaatttatttcagttactgtacttttcagctcc | |
| | | aagatttatctttggttcctttttataacgtctgtgtctt | |
| | | tattgatattctcattttgttcatatgtctctttcttcct | |
| | | ttagttctttgtccatgttttcctttagctctttgggctt | |
| | | atttaagacaattgtttaaagtctttgcatagtaagtcca | |
| | | atgtctgtgtttcttcagggatggttttcattattttgtt | |
| | | ttcaatgagccatactttcctgtgtctttgtatgctgtct | |
| | | ttttgttgttgaaaactgtatgtttgaacatcataacgtg | |
| | | gtggccctgaaaatcagatattccccccttcctgagagtt | |
| | | agttttatttttattattgaagattgtagcagtctattgc | |
| | | tacatgtgcagtcatttccaaactatttttgcaaagactg | |
| | | tattccttctgtgtgtcatcactgaagtctctgttcctta | |
| | | gtttgtgtttaatagtttgacatagatttccttgaaagga | |
| | | gttaaaactagcagaaaaatctctctcccagtctttccag | |
| | | tctttgtagattggttctgtgctgggcttttccattaata | |
| | | cttagccaggcttgtactgagcctaacaatcaggcccaaa | |
| | | agcgtagggtctttgcagatcttgtctgagcatgcttctt | |
| | | gctgtgtatgcacgtagttttctaaatctccctgtatgtg | |
| | | ctgttgaatattctaatttcccaaagaaactcctttgcag | |
| | | ctttttctcacagaacatagatggttttttggatatcttg | |
| | | accatagtctttcgacccaggtgtttgcggttgttagttc | |
| | | accttacacttttttcaagcattgcctactgcttacgatg | |
| | | agtgctctgtcaatcctttaagtagccccagacaggctac | |
| | | cagagacttaaacaagaatttgtaagttctgctcagcttc | |
| | | ctctagaaatggggatcagggtccaagacagaatgcagtt | |
| | | gctgatttcaagactgctgcaacaccagggagcttgtggg | |
| | | ggaagggcaagcagaaatgtcacaaagctttcttgccatt | |
| | | ttaaagttgcctgttcttgactcagcatttgcttcattgc | |
| | | tataaactttttactgtttttcagagttctgataaaattg | |
| | | gctatgcctgttcctgctttaaaaaatatatatatatttt | |
| | | ttagggattggggtctcactatactgaccaggctggtctt | |
| | | gaacttctggcctcaagccatcctctcatttcagcttccc | |
| | | aaagtgctgcaattacacgcgtgaaccaccacacccagcc | |
| | | cctgcttgtttttcaatgtgcctactccaccatgttgctc | |
| | | aagtatgtatattttctaaactaccttgtagtgttgtgat | |
| | | gggaaataaatccctgagccttttgaataactcagagaga | |
| | | tcaaaaacttagtttatcctattcgaaggattagaaaaat | |
| | | gatatatctttcactttttcagggataggctcctcattag | |
| | | aaggctcctatgtgccgatgctgtacaagacatttcattt | |
| | | ctcttaatgtttacaacaagcttgttgccaaggctgatct | |
| | | tgaactcctggcctcaaacgatcctcccagctcagtctca | |
| | | caaagtgttgggatgtctggccaactaatgactatcttaa | |
| | | ctcttgtgtttcaatgtttatgccttcttttatcttgact | |
| | | gattgtatgactatgtcttctagaacaatgttgaacagaa | |
| | | atggtgagagcagacatccttgctttaatatttcaccatt | |
| | | atatatgatgttaggtatagatttttctcacagatgcctt | |
| | | ttatcagattgaggaatttatattcctactttgccgaaag | |
| | | gtttttgtagtatgagggggtgctgaattttgtcaaacac | |
| | | tttttcggtaataattgagatgattggttctgcagtcatc | |
| | | gagatgtggattttctcctttattctgttcgtgagtgatt | |
| | | acactggttgactaatgttaaaacaaccttactttccagg | |
| | | aataaaccctattatcttttttataca | |
| PSMC4 | NM_153001.2 | tgcgggtacggacagcgcatgagcttatgttgagggcgga | 16 |
| | | gcccagaccagcccttcgtcctatcctgcccttccagcac | |
| | | ctctcagccgtaacttaaactacacttcccagaagcctcc | |
| | | tcagccagggacttccgttgtcgtcagcggaagcggtgac | |
| | | agatcatcccaggccacacagaggccggcttggtcactat | |
| | | ggaggagataggcatcttggtggagaaggctcaggatgag | |
| | | atcccagcactgtccgtgtcccggccccagaccggcctgt | |
| | | ccttcctgggccctgagcctgaggacctggaggacctgta | |
| | | cagccgctacaaggaggaggtgaagcgaatccaaagcatc | |
| | | ccgctggtcatcggacaatttctggaggctgtggatcaga | |
| | | atacagccatcgtgggctctaccacaggctccaactatta | |
| | | tgtgcgcatcctgagcaccatcgatcgggagctgctcaag | |
| | | cccaacgcctcagtggccctccacaagcacagcaatgcac | |
| | | tggtggacgtgctgccccccgaagccgacagcagcatcat | |
| | | gatgctcacctcagaccagaagccagatgtgatgtacgcg | |
| | | gacatcggaggcatggacatccagaagcaggaggtgcggg | |
| | | aggccgtggagctcccgctcacgcatttcgagctctacaa | |
| | | gcagatcggcatcgatcccccccgaggcgtcctcatgtat | |
| | | ggcccacctggctgtgggaagaccatgttggcaaaggcgg | |
| | | tggcacatcacacaacagctgcattcatccgggtcgtggg | |
| | | ctcggagtttgtacagaagtatctgggtgagggcccccgc | |
| | | atggtccgggatgtgttccgcctggccaaggagaatgcac | |
| | | ctgccatcatcttcatagacgagattgatgccatcgccac | |
| | | caagagattcgatgctcagacaggggccgacagggaggtt | |
| | | cagaggatcctgctggagctgctgaatcagatggatggat | |
| | | ttgatcagaatgtcaatgtcaaggtaatcatggccacaaa | |
| | | cagagcagacaccctggatccggccctgctacggccagga | |
| | | cggctggaccgtaaaattgaatttccacttcctgaccgcc | |
| | | gccagaagagattgattttctccactatcactagcaagat | |
| | | gaacctctctgaggaggttgacttggaagactatgtggcc | |
| | | cggccagataagatttcaggagctgatattaactccatct | |
| | | gtcaggagagtggaatgttggctgtccgtgaaaaccgcta | |
| | | cattgtcctggccaaggacttcgagaaagcatacaagact | |
| | | gtcatcaagaaggacgagcaggagcatgagttttacaagt | |
| | | gacccttcccttccctccaccacaccactcaggggctggg | |
| | | gcttctctcgcacccccagcacctctgtcccaaaacctca | |
| | | ttcccttttttctttacccaggattggtttcttcaataaa | |
| | | tagataagatcgaatccatttaatttcttcttagaagttt | |
| | | aactcctttggagaatgtgggccttgaataggatcctctg | |
| | | ggtccctcttaatctgacagatgagcagacgaggtgcatg | |
| | | gcctgggttgcagcttgagagaaccaaaatattcaaacca | |
| | | gatgacttccaaaatgtggggaaagggatggaaaatgaac | |
| | | ctgagatggagtccttaatcacgggataaagccctgtgca | |
| | | tctccctcatttcctacaggtaaaagacagtaaagaaatt | |
| | | caggtcacaggccttgggagttcataggaaggagatgtcc | |
| | | agtgctgtccagtagaacttt | |
| SF3A1 | NM_005877.5 | ggtcccggaagtgcgccagtcgtaccttcgcggccgcaac | 17 |
| | | tcgctcggccgccgccatcttgcgagctcgtcgtactgac | |
| | | cgagcggggaggctgtcttgaggcggcaccgctcaccgac | |
| | | accgaggcggactggcagccctgagcgtcgcagtcatgcc | |
| | | ggccggacccgtgcaggcggtgcccccgccgccgcccgtg | |
| | | cccacggagcccaaacagcccacagaagaagaagcatctt | |
| | | caaaggaggattctgcaccttctaagccagttgtggggat | |
| | | tatttaccctcctccagaggtcagaaatattgttgacaag | |
| | | actgccagctttgtggccagaaacgggcctgaatttgaag | |
| | | ctaggatccgacagaacgagatcaacaaccccaagttcaa | |
| | | ctttctgaaccccaatgacccttaccatgcctactaccgc | |
| | | cacaaggtcagcgagttcaaggaagggaaggctcaggagc | |
| | | cgtccgccgccatccccaaggtcatgcagcagcagcagca | |
| | | gaccacccagcagcagctgccccagaaggtccaagcccaa | |
| | | gtaatccaagagaccatcgtgcccaaagagcctcctcctg | |
| | | agtttgagttcattgctgatcctccctctatctcagcctt | |
| | | cgacttggatgtggtgaagctgacggctcagtttgtggcc | |
| | | aggaatgggcgccagtttctgacccagctgatgcagaaag | |
| | | agcagcgcaactaccagtttgactttctccgcccacagca | |
| | | cagcctcttcaactacttcacgaagctagtggaacagtac | |
| | | accaagatcttgattccacccaaaggtttattttcaaagc | |
| | | tcaagaaagaggctgaaaacccccgagaagttttggatca | |
| | | ggtgtgttaccgagtggaatgggccaaattccaggaacgt | |
| | | gagaggaagaaggaagaagaggagaaggagaaggagcggg | |
| | | tggcctatgctcagatcgactggcatgattttgtggtggt | |
| | | ggaaacagtggacttccaacccaatgagcaagggaacttc | |
| | | cctccccccaccacgccagaggagctgggggcccgaatcc | |
| | | tcattcaggagcgctatgaaaagtttggggagagtgagga | |
| | | agttgagatggaggtcgagtctgatgaggaggatgacaaa | |
| | | caggagaaggcggaggagcctccttcccagctggaccagg | |
| | | acacccaagtacaagatatggatgagggttcagatgatga | |
| | | agaagaagggcagaaagtgcccccacccccagagacaccc | |
| | | atgcctccacctctgcccccaactccagaccaagtcattg | |
| | | tccgcaaggattatgatcccaaagcctccaagcccttgcc | |
| | | tccagcccctgctccagatgagtatcttgtgtcccccatt | |
| | | actggggagaagatccccgccagcaaaatgcaggaacaca | |
| | | tgcgcattggacttcttgaccctcgctggctggagcagcg | |
| | | ggatcgctccatccgtgagaagcagagcgatgatgaggtg | |
| | | tacgcaccaggtctggatattgagagcagcttgaagcagt | |
| | | tggctgagcggcgtactgacatcttcggtgtagaggaaac | |
| | | agccattggtaagaagatcggtgaggaggagatccagaag | |
| | | ccagaggaaaaggtgacctgggatggccactcaggcagca | |
| | | tggcccggacccagcaggctgcccaggccaacatcaccct | |
| | | ccaggagcagattgaggccattcacaaggccaaaggcctg | |
| | | gtgccagaggatgacactaaagagaagattggccccagca | |
| | | agcccaatgaaatccctcaacagccaccgccaccatcttc | |
| | | agccaccaacatccccagctcggctccacccatcacttca | |
| | | gtgccccgaccacccacaatgccacctccagttcgtacta | |
| | | cagttgtctccgcagtacccgtcatgccccggcccccaat | |
| | | ggcatctgtggtccggctgcccccaggctcagtgatcgcc | |
| | | cccatgccgcccatcatccacgcgcccagaatcaacgtgg | |
| | | tgcccatgcctccctcggcccctcctattatggccccccg | |
| | | cccaccccccatgattgtgccaacagcctttgtgcctgct | |
| | | ccacctgtggcacctgtcccagctccagccccaatgcccc | |
| | | ctgtgcatcccccacctcccatggaagatgagcccacctc | |
| | | caaaaaactgaagacagaggacagcctcatgccagaggag | |
| | | gagttcctgcgcagaaacaagggtccagtgtccatcaaag | |
| | | tccaggtgcccaacatgcaggataagacggaatggaaact | |
| | | gaatgggcaggtgctggtcttcacccteccactcacggac | |
| | | caggtctctgtcattaaggtgaagattcatgaagccacag | |
| | | gcatgcctgcagggaaacagaagctacagtatgagggtat | |
| | | cttcatcaaagattccaactcactggcttactacaacatg | |
| | | gccaatggcgcagtcatccacctggccctcaaggagagag | |
| | | gcgggaggaagaagtagacaagaggaacctgctgtcaagt | |
| | | ccctgccattttgcctctcctgtctcccaccccctgcccc | |
| | | agacccaggagcccccctgaggctttgccttgcctgcata | |
| | | tttgtttcgctcttactcagtttgggaattcaaattgtcc | |
| | | tgcagaggttcattcccctgaccctttccccacattggta | |
| | | agagtagctgggttttctaagccactctctggaatctctt | |
| | | tgtgttagggtctcgatttgaggacattcatttcttcagc | |
| | | agcccattagcaactgagagcccagggatgtcctacagga | |
| | | tagtttcatagtgacaggtggcacttggctaatagaatat | |
| | | ggctgatattgtcattaatcattttgtaccttgacatggg | |
| | | ttgtctaataaaactcggacccttcttgtgaaatcagtta | |
| | | aataagacttgtctcggtcacctgtgccctgtccagactc | |
| | | gaggcagtggtaacactgcacagtgctatgtggcttctct | |
| | | ttgaggatttttgggttttgtaactaaattcttgctgccc | |
| | | tcatactttttatgtattagaatcatattcgtattgccct | |
| | | tttaaaacattgggatcctccaaaggcctgccccatgtat | |
| | | ttaacagtaatacaggaagcatggcaggcaccatgcaaac | |
| | | caaggatggatggtgcagtccctgtgtcagtgggcggtgg | |
| | | tttcctgctggcctggaatcactcatcacctgattgattg | |
| | | gctctgtggtcctgggcaggtgcctcataggtgtgtggat | |
| | | atgatgacgtttctttaaaatgtatgtatttaacaaatac | |
| | | ttaattgtattaaggtcatgtaccaaggatttgataaagt | |
| | | ttaaataatttactctctacttttatccattttatccatt | |
| | | ttaactcatgtaatcctcatgtgagtattcctgtttaaca | |
| | | cttgagtaaactgaggcacagagaacataagttgcatgcc | |
| | | atagtcacacactgtgaaagtgaaaagagaatgtgtgcaa | |
| | | aacacgtcacagtcctggtttctgagtaaaggcaggctgt | |
| | | tatctttagaatcaagctatcacagggagataggcaatgc | |
| | | tgtgggtgttggaggaaggtgagagcctgttgctaacaat | |
| | | ttcctggttttaaagctaaggctgattttattgggaagat | |
| | | ctcacatgtgtgtggcccctgagagttcccagtgcctttt | |
| | | atttgcagtccttccatttggacctcctagctgccccatc | |
| | | aggtcatctccagggctcagaggggtgagaccatttccca | |
| | | aggtcacagaaccagctctctagtcaccaccctgcctctc | |
| | | cctctcacccagagtcagtaccagttttatggctttatta | |
| | | caaactgctgggtccctcccattttcaacttgattgatgg | |
| | | gatgtcatcccttatcctgtctgacatttgcctctggcct | |
| | | ggttgctagaagtttgccccaggggcaagagttgaaattt | |
| | | ggcttcctgaggtgggctttgtggtttgcgtccctaaagt | |
| | | gagcccactactggttgcttgtccatggccaacaccagaa | |
| | | atcccctgagcactacctgggtctcattccaagaaggaag | |
| | | agggtcaggagacctggggagtctcatattccaagttctt | |
| | | ctttctttctgggagcagtgggcagttcatggtgttaggg | |
| | | cactcacccccacagactggcaaaccctgcaggacttccg | |
| | | tggctgaggctgtgaccggaggccaggaatgccgttgggt | |
| | | ggattgtgagtgaatgggccctttgagctgccctctagag | |
| | | agcaaatccagtttcctggagctcctgaatgaatatctgt | |
| | | actggctcgctcagatgcagaagctccattgaccatgagg | |
| | | ccttgtgaacatcagtggccacaggcccagtgtgctgctt | |
| | | ggcactgcactagtttaggacctgcagcatgtaggtagcg | |
| | | tcctagtgtttataatacaaagctgctctgcacagctttt | |
| | | ctgattcttcttgcaatctcctgaggattatctgccccat | |
| | | ttttaaaacgaggtggaatacccaaggtcatgtagccagt | |
| | | gagtgctctggaaagccaaagcagctcatcccttcctggg | |
| | | gaccacactgctctgctccaccagaccacactatgaaata | |
| | | ggaataagtgctcctgttgcaggactgctgggaaaacagg | |
| | | tggtgtgggacttaagtcaccataattttgaagacttgca | |
| | | tgcagagggctccaggaattgtagacattaaggaatttca | |
| | | ctttcagttctacccactacttaagtacttgtcatgtact | |
| | | cttagaggaggccagtaatgatcagaaccattttacttta | |
| | | aaattaataatattgtattagagaatatattaaatggtta | |
| | | tattgggttatgttaggatatatacttgaatggaaataca | |
| | | tgtactattagcaatcatatttcatttatccctgtaatta | |
| | | gacaagaaagcataatatagctctactcatgggtacacat | |
| | | accagtgtataagatttttagaagtttactttttaaaaat | |
| | | aaaagcaaaatgtaagatcttaaaaaaaaaaaaaaaaaa | |
| PUM1 | NM_001020658.1 | agtgggccgccatgttgtcggagtgaaaggtaagggggag | 18 |
| | | cgagagcgccagagagagaagatcggggggctgaaatcca | |
| | | tcttcatcctaccgctccgcccgtgttggtggaatgagcg | |
| | | ttgcatgtgtcttgaagagaaaagcagtgctttggcagga | |
| | | ctctttcagcccccacctgaaacatcaccctcaagaacca | |
| | | gctaatcccaacatgcctgttgttttgacatctggaacag | |
| | | ggtcgcaagcgcagccacaaccagctgcaaatcaggctct | |
| | | tgcagctgggactcactccagccctgtcccaggatctata | |
| | | ggagttgcaggccgttcccaggacgacgctatggtggact | |
| | | acttctttcagaggcagcatggtgagcagcttgggggagg | |
| | | aggaagtggaggaggcggctataataatagcaaacatcga | |
| | | tggcctactggggataacattcatgcagaacatcaggtgc | |
| | | gttccatggatgaactgaatcatgattttcaagcacttgc | |
| | | tctggagggaagagcgatgggagagcagctcttgccaggt | |
| | | aaaaagttttgggaaacagatgaatccagcaaagatggac | |
| | | caaaaggaatattcctgggtgatcaatggcgagacagtgc | |
| | | ctggggaacatcagatcattcagtttcccagccaatcatg | |
| | | gtgcagagaagacctggtcagagtttccatgtgaacagtg | |
| | | aggtcaattctgtactgtccccacgatcggagagtggggg | |
| | | actaggcgttagcatggtggagtatgtgttgagctcatcc | |
| | | ccgggcgattcctgtctaagaaaaggaggatttggcccaa | |
| | | gggatgcagacagtgatgaaaacgacaaaggtgaaaagaa | |
| | | gaacaagggtacgtttgatggagataagctaggagatttg | |
| | | aaggaggagggtgatgtgatggacaagaccaatggtttac | |
| | | cagtgcagaatgggattgatgcagacgtcaaagattttag | |
| | | ccgtacccctggtaattgccagaactctgctaatgaagtg | |
| | | gatcttctgggtccaaaccagaatggttctgagggcttag | |
| | | cccagctgaccagcaccaatggtgccaagcctgtggagga | |
| | | tttctccaacatggagtcccagagtgtccccttggacccc | |
| | | atggaacatgtgggcatggagcctcttcagtttgattatt | |
| | | caggcacgcaggtacctgtggactcagcagcagcaactgt | |
| | | gggactttttgactacaattctcaacaacagctgttccaa | |
| | | agacctaatgcgcttgctgtccagcagttgacagctgctc | |
| | | agcagcagcagtatgcactggcagctgctcatcagccgca | |
| | | catcggtttagctcccgctgcgtttgtccccaatccatac | |
| | | atcatcagcgctgctcccccagggacggacccctacacag | |
| | | ctggattggctgcagcagcgacactaggcccagctgtggt | |
| | | ccctcaccagtattatggagttactccctggggagtctac | |
| | | cctgccagtcttttccagcagcaagctgccgctgccgctg | |
| | | cagcaactaattcagctaatcaacagaccaccccacaggc | |
| | | tcagcaaggacagcagcaggttctccgtggaggagccagc | |
| | | caacgtcctttgaccccaaaccagaaccagcagggacagc | |
| | | aaacggatccccttgtggcagctgcagcagtgaattctgc | |
| | | ccttgcatttggacaaggtctggcagcaggcatgccaggt | |
| | | tatccggtgttggctcctgctgcttactatgaccaaactg | |
| | | gtgcccttgtagtgaatgcaggcgcgagaaatggtcttgg | |
| | | agctcctgttcgacttgtagctcctgccccagtcatcatt | |
| | | agttcctcagctgcacaagcagctgttgcagcagccgcag | |
| | | cttcagcaaatggagcagctggtggtcttgctggaacaac | |
| | | aaatggaccatttcgccctttaggaacacagcagcctcag | |
| | | ccccagccccagcagcagcccaataacaacctggcatcca | |
| | | gttctttctacggcaacaactctctgaacagcaattcaca | |
| | | gagcagctccctcttctcccagggctctgcccagcctgcc | |
| | | aacacatccttgggattcggaagtagcagttctctcggcg | |
| | | ccaccctgggatccgcccttggagggtttggaacagcagt | |
| | | tgcaaactccaacactggcagtggctcccgccgtgactcc | |
| | | ctgactggcagcagtgacctttataagaggacatcgagca | |
| | | gcttgacccccattggacacagtttttataacggccttag | |
| | | cttttcctcctctcctggacccgtgggcatgcctctccct | |
| | | agtcagggaccaggacattcacagacaccacctccttccc | |
| | | tctcttcacatggatcctcttcaagcttaaacctgggagg | |
| | | actcacgaatggcagtggaagatacatctctgctgctcca | |
| | | ggcgctgaagccaagtaccgcagtgcaagcagcgcctcca | |
| | | gcctcttcagcccgagcagcactcttttctcttcctctcg | |
| | | tttgcgatatggaatgtctgatgtcatgccttctggcagg | |
| | | agcaggcttttggaagattttcgaaacaaccggtacccca | |
| | | atttacaactgcgggagattgctggacatataatggaatt | |
| | | ttcccaagaccagcatgggtccagattcattcagctgaaa | |
| | | ctggagcgtgccacaccagctgagcgccagcttgtcttca | |
| | | atgaaatcctccaggctgcctaccaactcatggtggatgt | |
| | | gtttggtaattacgtcattcagaagttctttgaatttggc | |
| | | agtcttgaacagaagctggctttggcagaacggattcgag | |
| | | gccacgtcctgtcattggcactacagatgtatggctgccg | |
| | | tgttatccagaaagctcttgagtttattccttcagaccag | |
| | | caggtaattaatgagatggttcgggaactagatggccatg | |
| | | tcttgaagtgtgtgaaagatcagaatggcaatcacgtggt | |
| | | tcagaaatgcattgaatgtgtacagccccagtctttgcaa | |
| | | tttatcatcgatgcgtttaagggacaggtatttgccttat | |
| | | ccacacatccttatggctgccgagtgattcagagaatcct | |
| | | ggagcactgtctccctgaccagacactccctattttagag | |
| | | gagcttcaccagcacacagagcagcttgtacaggatcaat | |
| | | atggaaattatgtaatccaacatgtactggagcacggtcg | |
| | | tcctgaggataaaagcaaaattgtagcagaaatccgaggc | |
| | | aatgtacttgtattgagtcagcacaaatttgcaagcaatg | |
| | | ttgtggagaagtgtgttactcacgcctcacgtacggagcg | |
| | | cgctgtgctcatcgatgaggtgtgcaccatgaacgacggt | |
| | | ccccacagtgccttatacaccatgatgaaggaccagtatg | |
| | | ccaactacgtggtccagaagatgattgacgtggcggagcc | |
| | | aggccagcggaagatcgtcatgcataagatccggccccac | |
| | | atcgcaactcttcgtaagtacacctatggcaagcacattc | |
| | | tggccaagctggagaagtactacatgaagaacggtgttga | |
| | | cttagggcccatctgtggcccccctaatggtatcatctga | |
| | | ggcagtgtcacccgctgttccctcattcccgctgacctca | |
| | | ctggcccactggcaaatccaaccagcaaccagaaatgttc | |
| | | tagtgtagagtctgagacgggcaagtggttgctccaggat | |
| | | tactccctcctccaaaaaaggaatcaaatccacgagtgga | |
| | | aaagcctttgtaaatttaattttattacacataacatgta | |
| | | ctattttttttaattgactaattgccctgctgttttactg | |
| | | gtgtataggatacttgtacataggtaaccaatgtacatgg | |
| | | gaggccacatattttgttcactgttgtatctatatttcac | |
| | | atgtggaaactttcagggtggttggtttaacaaaaaaaaa | |
| | | aagctttaaaaaaaaaagaaaaaaaggaaaaggtttttag | |
| | | ctcatttgcctggccggcaagttttgcaaatagctcttcc | |
| | | ccacctcctcattttagtaaaaaacaaacaaaaacaaaaa | |
| | | aacctgagaagtttgaattgtagttaaatgaccccaaact | |
| | | ggcatttaacactgtttataaaaaatatatatatatatat | |
| | | atatatatataatgaaaaaggtttcagagttgctaaagct | |
| | | tcagtttgtgacattaagtttatgaaattctaaaaaatgc | |
| | | cttttttggagactatattatgctgaagaaggctgttcgt | |
| | | gaggaggagatgcgagcacccagaacgtcttttgaggctg | |
| | | ggcgggtgtgattgtttactgcctactggatttttttcta | |
| | | ttaacattgaaaggtaaaatctgattatttagcatgagaa | |
| | | aaaaaaatccaactctgcttttggtcttgcttctataaat | |
| | | atatagtgtatacttggtgtagactttgcatatatacaaa | |
| | | tttgtagtattttcttgttttgatgtctaatctgtatcta | |
| | | taatgtaccctagtagtcgaacatacttttgattgtacaa | |
| | | ttgtacatttgtatacctgtaatgtaaatgtggagaagtt | |
| | | tgaatcaacataaacacgttttttggtaagaaaagagaat | |
| | | tagccagccctgtgcattcagtgtatattctcacctttta | |
| | | tggtcgtagcatatagtgttgtatattgtaaattgtaatt | |
| | | tcaaccagaagtaaatttttttcttttgaaggaataaatg | |
| | | ttctttatacagcctagttaatgtttaaaaagaaaaaaat | |
| | | agcttggttttatttgtcatctagtctcaagtatagcgag | |
| | | attctttctaaatgttattcaagattgagttctcactagt | |
| | | gtttttttaatcctaaaaaagtaatgttttgattttgtga | |
| | | cagtcaaaaggacgtgcaaaagtctagccttgcccgagct | |
| | | ttccttacaatcagagcccctctcaccttgtaaagtgtga | |
| | | atcgcccttcccttttgtacagaagatgaactgtattttg | |
| | | cattttgtctacttgtaagtgaatgtaacatactgtcaat | |
| | | tttccttgtttgaatatagaattgtaacactacacggtgt | |
| | | acatttccagagccttgtgtatatttccaatgaacttttt | |
| | | tgcaagcacacttgtaaccatatgtgtataattaacaaac | |
| | | ctgtgtatgcttatgcctgggcaactattttttgtaactc | |
| | | ttgtgtagattgtctctaaacaatgtgtgatctttatttt | |
| | | gaaaaatacagaactttggaatctgaaaaaaaaaaaaaaa | |
| | | aaaaaaaaaaaaaaaa | |
| ACTB | NM_001101.4 | gagtgagcggcgcggggccaatcagcgtgcgccgttccga | 19 |
| | | aagttgccttttatggctcgagcggccgcggcggcgccct | |
| | | ataaaacccagcggcgcgacgcgccaccaccgccgagacc | |
| | | gcgtccgccccgcgagcacagagcctcgcctttgccgatc | |
| | | cgccgcccgtccacacccgccgccagctcaccatggatga | |
| | | tgatatcgccgcgctcgtcgtcgacaacggctccggcatg | |
| | | tgcaaggccggcttcgcgggcgacgatgccccccgggccg | |
| | | tcttcccctccatcgtggggcgccccaggcaccagggcgt | |
| | | gatggtgggcatgggtcagaaggattcctatgtgggcgac | |
| | | gaggcccagagcaagagaggcatcctcaccctgaagtacc | |
| | | ccatcgagcacggcatcgtcaccaactgggacgacatgga | |
| | | gaaaatctggcaccacaccttctacaatgagctgcgtgtg | |
| | | gctcccgaggagcaccccgtgctgctgaccgaggcccccc | |
| | | tgaaccccaaggccaaccgcgagaagatgacccagatcat | |
| | | gtttgagaccttcaacaccccagccatgtacgttgctatc | |
| | | caggctgtgctatccctgtacgcctctggccgtaccactg | |
| | | gcatcgtgatggactccggtgacggggtcacccacactgt | |
| | | gcccatctacgaggggtatgccctcccccatgccatcctg | |
| | | cgtctggacctggctggccgggacctgactgactacctca | |
| | | tgaagatcctcaccgagcgcggctacagcttcaccaccac | |
| | | ggccgagcgggaaatcgtgcgtgacattaaggagaagctg | |
| | | tgctacgtcgccctggacttcgagcaagagatggccacgg | |
| | | ctgcttccagctcctccctggagaagagctacgagctgcc | |
| | | tgacggccaggtcatcaccattggcaatgagcggttccgc | |
| | | tgccctgaggcactcttccagccttccttcctgggcatgg | |
| | | agtcctgtggcatccacgaaactaccttcaactccatcat | |
| | | gaagtgtgacgtggacatccgcaaagacctgtacgccaac | |
| | | acagtgctgtctggcggcaccaccatgtaccctggcattg | |
| | | ccgacaggatgcagaaggagatcactgccctggcacccag | |
| | | cacaatgaagatcaagatcattgctcctcctgagcgcaag | |
| | | tactccgtgtggatcggcggctccatcctggcctcgctgt | |
| | | ccaccttccagcagatgtggatcagcaagcaggagtatga | |
| | | cgagtccggcccctccatcgtccaccgcaaatgcttctag | |
| | | gcggactatgacttagttgcgttacaccctttcttgacaa | |
| | | aacctaacttgcgcagaaaacaagatgagattggcatggc | |
| | | tttatttgttttttttgttttgttttggtttttttttttt | |
| | | ttttggcttgactcaggatttaaaaactggaacggtgaag | |
| | | gtgacagcagtcggttggagcgagcatcccccaaagttca | |
| | | caatgtggccgaggactttgattgcacattgttgtttttt | |
| | | taatagtcattccaaatatgagatgcgttgttacaggaag | |
| | | tcccttgccatcctaaaagccaccccacttctctctaagg | |
| | | agaatggcccagtcctctcccaagtccacacaggggaggt | |
| | | gatagcattgctttcgtgtaaattatgtaatgcaaaattt | |
| | | ttttaatcttcgccttaatacttttttattttgttttatt | |
| | | ttgaatgatgagccttcgtgcccccccttccccctttttt | |
| | | gtcccccaacttgagatgtatgaaggcttttggtctccct | |
| | | gggagtgggtggaggcagccagggcttacctgtacactga | |
| | | cttgagaccagttgaataaaagtgcacaccttaaaaatga | |
| | | ggaaaaaaaaaaaaaaaaaa | |
| GAPD | NM_002046.6 | gctctctgctcctcctgttcgacagtcagccgcatcttct | 20 |
| | | tttgcgtcgccagccgagccacatcgctcagacaccatgg | |
| | | ggaaggtgaaggtcggagtcaacggatttggtcgtattgg | |
| | | gcgcctggtcaccagggctgcttttaactctggtaaagtg | |
| | | gatattgttgccatcaatgaccccttcattgacctcaact | |
| | | acatggtttacatgttccaatatgattccacccatggcaa | |
| | | attccatggcaccgtcaaggctgagaacgggaagcttgtc | |
| | | atcaatggaaatcccatcaccatcttccaggagcgagatc | |
| | | cctccaaaatcaagtggggcgatgctggcgctgagtacgt | |
| | | cgtggagtccactggcgtcttcaccaccatggagaaggct | |
| | | ggggctcatttgcaggggggagccaaaagggtcatcatct | |
| | | ctgccccctctgctgatgcccccatgttcgtcatgggtgt | |
| | | gaaccatgagaagtatgacaacagcctcaagatcatcagc | |
| | | aatgcctcctgcaccaccaactgcttagcacccctggcca | |
| | | aggtcatccatgacaactttggtatcgtggaaggactcat | |
| | | gaccacagtccatgccatcactgccacccagaagactgtg | |
| | | gatggcccctccgggaaactgtggcgtgatggccgcgggg | |
| | | ctctccagaacatcatccctgcctctactggcgctgccaa | |
| | | ggctgtgggcaaggtcatccctgagctgaacgggaagctc | |
| | | actggcatggccttccgtgtccccactgccaacgtgtcag | |
| | | tggtggacctgacctgccgtctagaaaaacctgccaaata | |
| | | tgatgacatcaagaaggtggtgaagcaggcgtcggagggc | |
| | | cccctcaagggcatcctgggctacactgagcaccaggtgg | |
| | | tctcctctgacttcaacagcgacacccactcctccacctt | |
| | | tgacgctggggctggcattgccctcaacgaccactttgtc | |
| | | aagctcatttcctggtatgacaacgaatttggctacagca | |
| | | acagggtggtggacctcatggcccacatggcctccaagga | |
| | | gtaagacccctggaccaccagccccagcaagagcacaaga | |
| | | ggaagagagagaccctcactgctggggagtccctgccaca | |
| | | ctcagtcccccaccacactgaatctcccctcctcacagtt | |
| | | gccatgtagaccccttgaagaggggaggggcctagggagc | |
| | | cgcaccttgtcatgtaccatcaataaagtaccctgtgctc | |
| | | aaccagttaaaaaaaaaaaaaaaaaaaaa | |
| GUSB | NM_000181.3 | gtcctcaaccaagatggcgcggatggcttcaggcgcatca | 21 |
| | | cgacaccggcgcgtcacgcgacccgccctacgggcacctc | |
| | | ccgcgcttttcttagcgccgcagacggtggccgagcgggg | |
| | | gaccgggaagcatggcccgggggtcggcggttgcctgggc | |
| | | ggcgctcgggccgttgttgtggggctgcgcgctggggctg | |
| | | cagggcgggatgctgtacccccaggagagcccgtcgcggg | |
| | | agtgcaaggagctggacggcctctggagcttccgcgccga | |
| | | cttctctgacaaccgacgccggggcttcgaggagcagtgg | |
| | | taccggcggccgctgtgggagtcaggccccaccgtggaca | |
| | | tgccagttccctccagcttcaatgacatcagccaggactg | |
| | | gcgtctgcggcattttgtcggctgggtgtggtacgaacgg | |
| | | gaggtgatcctgccggagcgatggacccaggacctgcgca | |
| | | caagagtggtgctgaggattggcagtgcccattcctatgc | |
| | | catcgtgtgggtgaatggggtcgacacgctagagcatgag | |
| | | gggggctacctccccttcgaggccgacatcagcaacctgg | |
| | | tccaggtggggcccctgccctcccggctccgaatcactat | |
| | | cgccatcaacaacacactcacccccaccaccctgccacca | |
| | | gggaccatccaatacctgactgacacctccaagtatccca | |
| | | agggttactttgtccagaacacatattttgactttttcaa | |
| | | ctacgctggactgcagcggtctgtacttctgtacacgaca | |
| | | cccaccacctacatcgatgacatcaccgtcaccaccagcg | |
| | | tggagcaagacagtgggctggtgaattaccagatctctgt | |
| | | caagggcagtaacctgttcaagttggaagtgcgtcttttg | |
| | | gatgcagaaaacaaagtcgtggcgaatgggactgggaccc | |
| | | agggccaacttaaggtgccaggtgtcagcctctggtggcc | |
| | | gtacctgatgcacgaacgccctgcctatctgtattcattg | |
| | | gaggtgcagctgactgcacagacgtcactggggcctgtgt | |
| | | ctgacttctacacactccctgtggggatccgcactgtggc | |
| | | tgtcaccaagagccagttcctcatcaatgggaaacctttc | |
| | | tatttccacggtgtcaacaagcatgaggatgcggacatcc | |
| | | gagggaagggcttcgactggccgctgctggtgaaggactt | |
| | | caacctgcttcgctggcttggtgccaacgctttccgtacc | |
| | | agccactacccctatgcagaggaagtgatgcagatgtgtg | |
| | | accgctatgggattgtggtcatcgatgagtgtcccggcgt | |
| | | gggcctggcgctgccgcagttcttcaacaacgtttctctg | |
| | | catcaccacatgcaggtgatggaagaagtggtgcgtaggg | |
| | | acaagaaccaccccgcggtcgtgatgtggtctgtggccaa | |
| | | cgagcctgcgtcccacctagaatctgctggctactacttg | |
| | | aagatggtgatcgctcacaccaaatccttggacccctccc | |
| | | ggcctgtgacctttgtgagcaactctaactatgcagcaga | |
| | | caagggggctccgtatgtggatgtgatctgtttgaacagc | |
| | | tactactcttggtatcacgactacgggcacctggagttga | |
| | | ttcagctgcagctggccacccagtttgagaactggtataa | |
| | | gaagtatcagaagcccattattcagagcgagtatggagca | |
| | | gaaacgattgcagggtttcaccaggatccacctctgatgt | |
| | | tcactgaagagtaccagaaaagtctgctagagcagtacca | |
| | | tctgggtctggatcaaaaacgcagaaaatacgtggttgga | |
| | | gagctcatttggaattttgccgatttcatgactgaacagt | |
| | | caccgacgagagtgctggggaataaaaaggggatcttcac | |
| | | tcggcagagacaaccaaaaagtgcagcgttccttttgcga | |
| | | gagagatactggaagattgccaatgaaaccaggtatcccc | |
| | | actcagtagccaagtcacaatgtttggaaaacagcctgtt | |
| | | tacttgagcaagactgataccacctgcgtgtcccttcctc | |
| | | cccgagtcagggcgacttccacagcagcagaacaagtgcc | |
| | | tcctggactgttcacggcagaccagaacgtttctggcctg | |
| | | ggttttgtggtcatctattctagcagggaacactaaaggt | |
| | | ggaaataaaagattttctattatggaaataaagagttggc | |
| | | atgaaagtggctactgaaaaaaaaaaaaaaaaaaaaaaaa | |
| | | a | |
| RPLP0 | NM_001002.3 | gtctgacgggcgatggcgcagccaatagacaggagcgcta | 22 |
| | | tccgcggtttctgattggctactttgttcgcattataaaa | |
| | | ggcacgcgcgggcgcgaggcccttctctcgccaggcgtcc | |
| | | tcgtggaagtgacatcgtctttaaaccctgcgtggcaatc | |
| | | cctgacgcaccgccgtgatgcccagggaagacagggcgac | |
| | | ctggaagtccaactacttccttaagatcatccaactattg | |
| | | gatgattatccgaaatgtttcattgtgggagcagacaatg | |
| | | tgggctccaagcagatgcagcagatccgcatgtcccttcg | |
| | | cgggaaggctgtggtgctgatgggcaagaacaccatgatg | |
| | | cgcaaggccatccgagggcacctggaaaacaacccagctc | |
| | | tggagaaactgctgcctcatatccgggggaatgtgggctt | |
| | | tgtgttcaccaaggaggacctcactgagatcagggacatg | |
| | | ttgctggccaataaggtgccagctgctgcccgtgctggtg | |
| | | ccattgccccatgtgaagtcactgtgccagcccagaacac | |
| | | tggtctcgggcccgagaagacctcctttttccaggcttta | |
| | | ggtatcaccactaaaatctccaggggcaccattgaaatcc | |
| | | tgagtgatgtgcagctgatcaagactggagacaaagtggg | |
| | | agccagcgaagccacgctgctgaacatgctcaacatctcc | |
| | | cccttctcctttgggctggtcatccagcaggtgttcgaca | |
| | | atggcagcatctacaaccctgaagtgcttgatatcacaga | |
| | | ggaaactctgcattctcgcttcctggagggtgtccgcaat | |
| | | gttgccagtgtctgtctgcagattggctacccaactgttg | |
| | | catcagtaccccattctatcatcaacgggtacaaacgagt | |
| | | cctggccttgtctgtggagacggattacaccttcccactt | |
| | | gctgaaaaggtcaaggccttcttggctgatccatctgcct | |
| | | ttgtggctgctgcccctgtggctgctgccaccacagctgc | |
| | | tcctgctgctgctgcagccccagctaaggttgaagccaag | |
| | | gaagagtcggaggagtcggacgaggatatgggatttggtc | |
| | | tctttgactaatcaccaaaaagcaaccaacttagccagtt | |
| | | ttatttgcaaaacaaggaaataaaggcttacttctttaaa | |
| | | aagtaaaaaaaaaaaaaaaaaaaaaaaaa | |
| TFRC | NM_003234.3 | agagcgtcgggatatcgggtggcggctcgggacggaggac | 23 |
| | | gcgctagtgtgagtgcgggcttctagaactacaccgaccc | |
| | | tcgtgtcctcccttcatcctgcggggctggctggagcggc | |
| | | cgctccggtgctgtccagcagccatagggagccgcacggg | |
| | | gagcgggaaagcggtcgcggccccaggcggggcggccggg | |
| | | atggagcggggccgcgagcctgtggggaaggggctgtggc | |
| | | ggcgcctcgagcggctgcaggttcttctgtgtggcagttc | |
| | | agaatgatggatcaagctagatcagcattctctaacttgt | |
| | | ttggtggagaaccattgtcatatacccggttcagcctggc | |
| | | tcggcaagtagatggcgataacagtcatgtggagatgaaa | |
| | | cttgctgtagatgaagaagaaaatgctgacaataacacaa | |
| | | aggccaatgtcacaaaaccaaaaaggtgtagtggaagtat | |
| | | ctgctatgggactattgctgtgatcgtctttttcttgatt | |
| | | ggatttatgattggctacttgggctattgtaaaggggtag | |
| | | aaccaaaaactgagtgtgagagactggcaggaaccgagtc | |
| | | tccagtgagggaggagccaggagaggacttccctgcagca | |
| | | cgtcgcttatattgggatgacctgaagagaaagttgtcgg | |
| | | agaaactggacagcacagacttcaccggcaccatcaagct | |
| | | gctgaatgaaaattcatatgtccctcgtgaggctggatct | |
| | | caaaaagatgaaaatcttgcgttgtatgttgaaaatcaat | |
| | | ttcgtgaatttaaactcagcaaagtctggcgtgatcaaca | |
| | | ttttgttaagattcaggtcaaagacagcgctcaaaactcg | |
| | | gtgatcatagttgataagaacggtagacttgtttacctgg | |
| | | tggagaatcctgggggttatgtggcgtatagtaaggctgc | |
| | | aacagttactggtaaactggtccatgctaattttggtact | |
| | | aaaaaagattttgaggatttatacactcctgtgaatggat | |
| | | ctatagtgattgtcagagcagggaaaatcacctttgcaga | |
| | | aaaggttgcaaatgctgaaagcttaaatgcaattggtgtg | |
| | | ttgatatacatggaccagactaaatttcccattgttaacg | |
| | | cagaactttcattctttggacatgctcatctggggacagg | |
| | | tgacccttacacacctggattcccttccttcaatcacact | |
| | | cagtttccaccatctcggtcatcaggattgcctaatatac | |
| | | ctgtccagacaatctccagagctgctgcagaaaagctgtt | |
| | | tgggaatatggaaggagactgtccctctgactggaaaaca | |
| | | gactctacatgtaggatggtaacctcagaaagcaagaatg | |
| | | tgaagctcactgtgagcaatgtgctgaaagagataaaaat | |
| | | tcttaacatctttggagttattaaaggctttgtagaacca | |
| | | gatcactatgttgtagttggggcccagagagatgcatggg | |
| | | gccctggagctgcaaaatccggtgtaggcacagctctcct | |
| | | attgaaacttgcccagatgttctcagatatggtcttaaaa | |
| | | gatgggtttcagcccagcagaagcattatctttgccagtt | |
| | | ggagtgctggagactttggatcggttggtgccactgaatg | |
| | | gctagagggatacctttcgtccctgcatttaaaggctttc | |
| | | acttatattaatctggataaagcggttcttggtaccagca | |
| | | acttcaaggtttctgccagcccactgttgtatacgcttat | |
| | | tgagaaaacaatgcaaaatgtgaagcatccggttactggg | |
| | | caatttctatatcaggacagcaactgggccagcaaagttg | |
| | | agaaactcactttagacaatgctgctttccctttccttgc | |
| | | atattctggaatcccagcagtttctttctgtttttgcgag | |
| | | gacacagattatccttatttgggtaccaccatggacacct | |
| | | ataaggaactgattgagaggattcctgagttgaacaaagt | |
| | | ggcacgagcagctgcagaggtcgctggtcagttcgtgatt | |
| | | aaactaacccatgatgttgaattgaacctggactatgaga | |
| | | ggtacaacagccaactgctttcatttgtgagggatctgaa | |
| | | ccaatacagagcagacataaaggaaatgggcctgagttta | |
| | | cagtggctgtattctgctcgtggagacttcttccgtgcta | |
| | | cttccagactaacaacagatttcgggaatgctgagaaaac | |
| | | agacagatttgtcatgaagaaactcaatgatcgtgtcatg | |
| | | agagtggagtatcacttcctctctccctacgtatctccaa | |
| | | aagagtctcctttccgacatgtcttctggggctccggctc | |
| | | tcacacgctgccagctttactggagaacttgaaactgcgt | |
| | | aaacaaaataacggtgcttttaatgaaacgctgttcagaa | |
| | | accagttggctctagctacttggactattcagggagctgc | |
| | | aaatgccctctctggtgacgtttgggacattgacaatgag | |
| | | ttttaaatgtgatacccatagcttccatgagaacagcagg | |
| | | gtagtctggtttctagacttgtgctgatcgtgctaaattt | |
| | | tcagtagggctacaaaacctgatgttaaaattccatccca | |
| | | tcatcttggtactactagatgtctttaggcagcagctttt | |
| | | aatacagggtagataacctgtacttcaagttaaagtgaat | |
| | | aaccacttaaaaaatgtccatgatggaatattcccctatc | |
| | | tctagaattttaagtgctttgtaatgggaactgcctcttt | |
| | | cctgttgttgttaatgaaaatgtcagaaaccagttatgtg | |
| | | aatgatctctctgaatcctaagggctggtctctgctgaag | |
| | | gttgtaagtggtcgcttactttgagtgatcctccaacttc | |
| | | atttgatgctaaataggagataccaggttgaaagaccttc | |
| | | tccaaatgagatctaagcctttccataaggaatgtagctg | |
| | | gtttcctcattcctgaaagaaacagttaactttcagaaga | |
| | | gatgggcttgttttcttgccaatgaggtctgaaatggagg | |
| | | tccttctgctggataaaatgaggttcaactgttgattgca | |
| | | ggaataaggccttaatatgttaacctcagtgtcatttatg | |
| | | aaaagaggggaccagaagccaaagacttagtatattttct | |
| | | tttcctctgtcccttcccccataagcctccatttagttct | |
| | | ttgttatttttgtttcttccaaagcacattgaaagagaac | |
| | | cagtttcaggtgtttagttgcagactcagtttgtcagact | |
| | | ttaaagaataatatgctgccaaattttggccaaagtgtta | |
| | | atcttaggggagagctttctgtccttttggcactgagata | |
| | | tttattgtttatttatcagtgacagagttcactataaatg | |
| | | gtgtttttttaatagaatataattatcggaagcagtgcct | |
| | | tccataattatgacagttatactgtcggttttttttaaat | |
| | | aaaagcagcatctgctaataaaacccaacagatactggaa | |
| | | gttttgcatttatggtcaacacttaagggttttagaaaac | |
| | | agccgtcagccaaatgtaattgaataaagttgaagctaag | |
| | | atttagagatgaattaaatttaattaggggttgctaagaa | |
| | | gcgagcactgaccagataagaatgctggttttcctaaatg | |
| | | cagtgaattgtgaccaagttataaatcaatgtcacttaaa | |
| | | ggctgtggtagtactcctgcaaaattttatagctcagttt | |
| | | atccaaggtgtaactctaattcccattttgcaaaatttcc | |
| | | agtacctttgtcacaatcctaacacattatcgggagcagt | |
| | | gtcttccataatgtataaagaacaaggtagtttttaccta | |
| | | ccacagtgtctgtatcggagacagtgatctccatatgtta | |
| | | cactaagggtgtaagtaattatcgggaacagtgtttccca | |
| | | taattttcttcatgcaatgacatcttcaaagcttgaagat | |
| | | cgttagtatctaacatgtatcccaactcctataattccct | |
| | | atcttttagttttagttgcagaaacattttgtggtcatta | |
| | | agcattgggtgggtaaattcaaccactgtaaaatgaaatt | |
| | | actacaaaatttgaaatttagcttgggtttttgttacctt | |
| | | tatggtttctccaggtcctctacttaatgagatagtagca | |
| | | tacatttataatgtttgctattgacaagtcattttaactt | |
| | | tatcacattatttgcatgttacctcctataaacttagtgc | |
| | | ggacaagttttaatccagaattgaccttttgacttaaagc | |
| | | agagggactttgtatagaaggtttgggggctgtggggaag | |
| | | gagagtcccctgaaggtctgacacgtctgcctacccattc | |
| | | gtggtgatcaattaaatgtaggtatgaataagttcgaagc | |
| | | tccgtgagtgaaccatcattataaacgtgatgatcagctg | |
| | | tttgtcatagggcagttggaaacggcctcctagggaaaag | |
| | | ttcatagggtctcttcaggttcttagtgtcacttacctag | |
| | | atttacagcctcacttgaatgtgtcactactcacagtctc | |
| | | tttaatcttcagttttatctttaatctcctcttttatctt | |
| | | ggactgacatttagcgtagctaagtgaaaaggtcatagct | |
| | | gagattcctggttcgggtgttacgcacacgtacttaaatg | |
| | | aaagcatgtggcatgttcatcgtataacacaatatgaata | |
| | | cagggcatgcattttgcagcagtgagtctcttcagaaaac | |
| | | ccttttctacagttagggttgagttacttcctatcaagcc | |
| | | agtacgtgctaacaggctcaatattcctgaatgaaatatc | |
| | | agactagtgacaagctcctggtcttgagatgtcttctcgt | |
| | | taaggagatgggccttttggaggtaaaggataaaatgaat | |
| | | gagttctgtcatgattcactattctagaacttgcatgacc | |
| | | tttactgtgttagctctttgaatgttcttgaaattttaga | |
| | | ctttctttgtaaacaaatgatatgtccttatcattgtata | |
| | | aaagctgttatgtgcaacagtgtggagattccttgtctga | |
| | | tttaataaaatacttaaacactgaaaaaaaaaaa | |
| 18S | X03205.1 | tacctggttgatcctgccagtagcatatgcttgtctcaaa | 24 |
| | | gattaagccatgcatgtctaagtacgcacggccggtacag | |
| | | tgaaactgcgaatggctcattaaatcagttatggttcctt | |
| | | tggtcgctcgctcctctcccacttggataactgtggtaat | |
| | | tctagagctaatacatgccgacgggcgctgacccccttcg | |
| | | cgggggggatgcgtgcatttatcagatcaaaaccaacccg | |
| | | gtcagcccctctccggccccggccggggggcgggcgccgg | |
| | | cggctttggtgactctagataacctcgggccgatcgcacg | |
| | | ccccccgtggcggcgacgacccattcgaacgtctgcccta | |
| | | tcaactttcgatggtagtcgccgtgcctaccatggtgacc | |
| | | acgggtgacggggaatcagggttcgattccggagagggag | |
| | | cctgagaaacggctaccacatccaaggaaggcagcaggcg | |
| | | cgcaaattacccactcccgacccggggaggtagtgacgaa | |
| | | aaataacaatacaggactctttcgaggccctgtaattgga | |
| | | atgagtccactttaaatcctttaacgaggatccattggag | |
| | | ggcaagtctggtgccagcagccgcggtaattccagctcca | |
| | | atagcgtatattaaagttgctgcagttaaaaagctcgtag | |
| | | ttggatcttgggagcgggcgggcggtccgccgcgaggcga | |
| | | gccaccgcccgtccccgccccttgcctctcggcgccccct | |
| | | cgatgctcttagctgagtgtcccgcggggcccgaagcgtt | |
| | | tactttgaaaaaattagagtgttcaaagcaggcccgagcc | |
| | | gcctggataccgcagctaggaataatggaataggaccgcg | |
| | | gttctattttgttggttttcggaactgaggccatgattaa | |
| | | gagggacggccgggggcattcgtattgcgccgctagaggt | |
| | | gaaattcttggaccggcgcaagacggaccagagcgaaagc | |
| | | atttgccaagaatgttttcattaatcaagaacgaaagtcg | |
| | | gaggttcgaagacgatcagataccgtcgtagttccgacca | |
| | | taaacgatgccgaccggcgatgcggcggcgttattcccat | |
| | | gaccegccgggcagcttccgggaaaccaaagtctttgggt | |
| | | tccggggggagtatggttgcaaagctgaaacttaaaggaa | |
| | | ttgacggaagggcaccaccaggagtggagcctgcggctta | |
| | | atttgactcaacacgggaaacctcacccggcccggacacg | |
| | | gacaggattgacagattgatagctctttctcgattccgtg | |
| | | ggtggtggtgcatggccgttcttagttggtggagcgattt | |
| | | gtctggttaattccgataacgaacgagactctggcatgct | |
| | | aactagttacgcgacccccgagcggtcggcgtcccccaac | |
| | | ttcttagagggacaagtggcgttcagccacccgagattga | |
| | | gcaataacaggtctgtgatgcccttagatgtccggggctg | |
| | | cacgcgcgctacactgactggctcagcgtgtgcctaccct | |
| | | acgccggcaggcgcgggtaacccgttgaaccccattcgtg | |
| | | atggggatcggggattgcaattattccccatgaacgagga | |
| | | attcccagtaagtgcgggtcataagcttgcgttgattaag | |
| | | tccctgccctttgtacacaccgcccgtcgctactaccgat | |
| | | tggatggtttagtgaggccctcggatcggccccgccgggg | |
| | | tcggcccacggccctggcggagcgctgagaagacggtcga | |
| | | acttgactatctagaggaagtaaaagtcgtaacaaggttt | |
| | | ccgtaggtgaacctgcggaaggatcatta | |
| PPIA | NM_021130.4 | ggggccgaacgtggtataaaaggggcgggaggccaggctc | 25 |
| | | gtgccgttttgcagacgccaccgccgaggaaaaccgtgta | |
| | | ctattagccatggtcaaccccaccgtgttcttcgacattg | |
| | | ccgtcgacggcgagcccttgggccgcgtctcctttgagct | |
| | | gtttgcagacaaggtcccaaagacagcagaaaattttcgt | |
| | | gctctgagcactggagagaaaggatttggttataagggtt | |
| | | cctgctttcacagaattattccagggtttatgtgtcaggg | |
| | | tggtgacttcacacgccataatggcactggtggcaagtcc | |
| | | atctatggggagaaatttgaagatgagaacttcatcctaa | |
| | | agcatacgggtcctggcatcttgtccatggcaaatgctgg | |
| | | acccaacacaaatggttcccagtttttcatctgcactgcc | |
| | | aagactgagtggttggatggcaagcatgtggtgtttggca | |
| | | aagtgaaagaaggcatgaatattgtggaggccatggagcg | |
| | | ctttgggtccaggaatggcaagaccagcaagaagatcacc | |
| | | attgctgactgtggacaactcgaataagtttgacttgtgt | |
| | | tttatcttaaccaccagatcattccttctgtagctcagga | |
| | | gagcacccctccaccccatttgctcgcagtatcctagaat | |
| | | ctttgtgctctcgctgcagttccctttgggttccatgttt | |
| | | tccttgttccctcccatgcctagctggattgcagagttaa | |
| | | gtttatgattatgaaataaaaactaaataacaattgtcct | |
| | | cgtttgagttaagagtgttgatgtaggctttattttaagc | |
| | | agtaatgggttacttctgaaacatcacttgtttgcttaat | |
| | | tctacacagtacttagattttttttactttccagtcccag | |
| | | gaagtgtcaatgtttgttgagtggaatattgaaaatgtag | |
| | | gcagcaactgggcatggtggctcactgtctgtaatgtatt | |
| | | acctgaggcagaagaccacctgagggtaggagtcaagatc | |
| | | agcctgggcaacatagtgagacgctgtctctacaaaaaat | |
| | | aattagcctggcctggtggtgcatgcctagtcctagctga | |
| | | tctggaggctgacgtgggaggattgcttgagcctagagtg | |
| | | agctattatcatgccactgtacagcctgggtgttcacaga | |
| | | tcttgtgtctcaaaggtaggcagaggcaggaaaagcaagg | |
| | | agccagaattaagaggttgggtcagtctgcagtgagttca | |
| | | tgcatttagaggtgttcttcaagatgactaatgtcaaaaa | |
| | | ttgagacatctgttgcggttttttttttttttttttcccc | |
| | | tggaatgcagtggcgtgatctcagctcactgcagcctccg | |
| | | cctcctgggttcaagtgattctagtgcctcagcctcctga | |
| | | gtagctgggataatgggcgtgtgccaccatgcccagctaa | |
| | | tttttgtatttttagtatagatggggtttcatcattttga | |
| | | ccaggctggtctcaaactcttgacctcagctgatgcgcct | |
| | | gccttggcctcccaaactgctgagattacagatgtgagcc | |
| | | accgcaccctacctcattttctgtaacaaagctaagcttg | |
| | | aacactgttgatgttcttgagggaagcatattgggcttta | |
| | | ggctgtaggtcaagtttatacatcttaattatggtggaat | |
| | | tcctatgtagagtctaaaaagccaggtacttggtgctaca | |
| | | gtcagtctccctgcagagggttaaggcgcagactacctgc | |
| | | agtgaggaggtactgcttgtagcatatagagcctctccct | |
| | | agctttggttatggaggctttgaggttttgcaaacctgac | |
| | | caatttaagccataagatctggtcaaagggatacccttcc | |
| | | cactaaggacttggtttctcaggaaattatatgtacagtg | |
| | | cttgctggcagttagatgtcaggacaatctaagctgagaa | |
| | | aaccccttctctgcccaccttaacagacctctagggttct | |
| | | taacccagcaatcaagtttgcctatcctagaggtggcgga | |
| | | tttgatcatttggtgtgttgggcaatttttgttttactgt | |
| | | ctggttccttctgcgtgaattaccaccaccaccacttgtg | |
| | | catctcagtcttgtgtgttgtctggttacgtattccctgg | |
| | | gtgataccattcaatgtcttaatgtacttgtggctcagac | |
| | | ctgagtgcaaggtggaaataaacatcaaacatcttttcat | |
| | | tatcccta | |
| PGK1 | NM_000291.3 | gagagcagcggccgggaaggggcggtgcgggaggcggggt | 26 |
| | | gtggggcggtagtgtgggccctgttcctgcccgcgcggtg | |
| | | ttccgcattctgcaagcctccggagcgcacgtcggcagtc | |
| | | ggctccctcgttgaccgaatcaccgacctctctccccagc | |
| | | tgtatttccaaaatgtcgctttctaacaagctgacgctgg | |
| | | acaagctggacgttaaagggaagcgggtcgttatgagagt | |
| | | cgacttcaatgttcctatgaagaacaaccagataacaaac | |
| | | aaccagaggattaaggctgctgtcccaagcatcaaattct | |
| | | gcttggacaatggagccaagtcggtagtccttatgagcca | |
| | | cctaggccggcctgatggtgtgcccatgcctgacaagtac | |
| | | tccttagagccagttgctgtagaactcaaatctctgctgg | |
| | | gcaaggatgttctgttcttgaaggactgtgtaggcccaga | |
| | | agtggagaaagcctgtgccaacccagctgctgggtctgtc | |
| | | atcctgctggagaacctccgctttcatgtggaggaagaag | |
| | | ggaagggaaaagatgcttctgggaacaaggttaaagccga | |
| | | gccagccaaaatagaagctttccgagcttcactttccaag | |
| | | ctaggggatgtctatgtcaatgatgcttttggcactgctc | |
| | | acagagcccacagctccatggtaggagtcaatctgccaca | |
| | | gaaggctggtgggtttttgatgaagaaggagctgaactac | |
| | | tttgcaaaggccttggagagcccagagcgacccttcctgg | |
| | | ccatcctgggcggagctaaagttgcagacaagatccagct | |
| | | catcaataatatgctggacaaagtcaatgagatgattatt | |
| | | ggtggtggaatggcttttaccttccttaaggtgctcaaca | |
| | | acatggagattggcacttctctgtttgatgaagagggagc | |
| | | caagattgtcaaagacctaatgtccaaagctgagaagaat | |
| | | ggtgtgaagattaccttgcctgttgactttgtcactgctg | |
| | | acaagtttgatgagaatgccaagactggccaagccactgt | |
| | | ggcttctggcatacctgctggctggatgggcttggactgt | |
| | | ggtcctgaaagcagcaagaagtatgctgaggctgtcactc | |
| | | gggctaagcagattgtgtggaatggtcctgtgggggtatt | |
| | | tgaatgggaagcttttgcccggggaaccaaagctctcatg | |
| | | gatgaggtggtgaaagccacttctaggggctgcatcacca | |
| | | tcataggtggtggagacactgccacttgctgtgccaaatg | |
| | | gaacacggaggataaagtcagccatgtgagcactgggggt | |
| | | ggtgccagtttggagctcctggaaggtaaagtccttcctg | |
| | | gggtggatgctctcagcaatatttagtactttcctgcctt | |
| | | ttagttcctgtgcacagcccctaagtcaacttagcatttt | |
| | | ctgcatctccacttggcattagctaaaaccttccatgtca | |
| | | agattcagctagtggccaagagatgcagtgccaggaaccc | |
| | | ttaaacagttgcacagcatctcagctcatcttcactgcac | |
| | | cctggatttgcatacattcttcaagatcccatttgaattt | |
| | | tttagtgactaaaccattgtgcattctagagtgcatatat | |
| | | ttatattttgcctgttaaaaagaaagtgagcagtgttagc | |
| | | ttagttctcttttgatgtaggttattatgattagctttgt | |
| | | cactgtttcactactcagcatggaaacaagatgaaattcc | |
| | | atttgtaggtagtgagacaaaattgatgatccattaagta | |
| | | aacaataaaagtgtccattgaaaccgtgattttttttttt | |
| | | ttcctgtcatactttgttaggaagggtgagaatagaatct | |
| | | tgaggaacggatcagatgtctatattgctgaatgcaagaa | |
| | | gtggggcagcagcagtggagagatgggacaattagataaa | |
| | | tgtccattctttatcaagggcctactttatggcagacatt | |
| | | gtgctagtgcttttattctaacttttatttttatcagtta | |
| | | cacatgatcataatttaaaaagtcaaggcttataacaaaa | |
| | | aagccccagcccattcctcccattcaagattcccactccc | |
| | | cagaggtgaccactttcaactcttgagtttttcaggtata | |
| | | tacctccatgtttctaagtaatatgcttatattgttcact | |
| | | tcttttttttttattttttaaagaaatctatttcatacca | |
| | | tggaggaaggctctgttccacatatatttccacttcttca | |
| | | ttctctcggtatagttttgtcacaattatagattagatca | |
| | | aaagtctacataactaatacagctgagctatgtagtatgc | |
| | | tatgattaaatttacttatgtaaaaaaaaaaaaaaaaaa | |
| RPL13A | NM_012423.3 | cacttctgccgcccctgtttcaagggataagaaaccctgc | 27 |
| | | gacaaaacctcctccttttccaagcggctgccgaagatgg | |
| | | cggaggtgcaggtcctggtgcttgatggtcgaggccatct | |
| | | cctgggccgcctggcggccatcgtggctaaacaggtactg | |
| | | ctgggccggaaggtggtggtcgtacgctgtgaaggcatca | |
| | | acatttctggcaatttctacagaaacaagttgaagtacct | |
| | | ggctttcctccgcaagcggatgaacaccaacccttcccga | |
| | | ggcccctaccacttccgggcccccagccgcatcttctggc | |
| | | ggaccgtgcgaggtatgctgccccacaaaaccaagcgagg | |
| | | ccaggccgctctggaccgtctcaaggtgtttgacggcatc | |
| | | ccaccgccctacgacaagaaaaagcggatggtggttcctg | |
| | | ctgccctcaaggtcgtgcgtctgaagcctacaagaaagtt | |
| | | tgcctatctggggcgcctggctcacgaggttggctggaag | |
| | | taccaggcagtgacagccaccctggaggagaagaggaaag | |
| | | agaaagccaagatccactaccggaagaagaaacagctcat | |
| | | gaggctacggaaacaggccgagaagaacgtggagaagaaa | |
| | | attgacaaatacacagaggtcctcaagacccacggactcc | |
| | | tggtctgagcccaataaagactgttaattcctcatgcgtt | |
| | | gcctgcccttcctccattgttgccctggaatgtacgggac | |
| | | ccaggggcagcagcagtccaggtgccacaggcagccctgg | |
| | | gacataggaagctgggagcaaggaaagggtcttagtcact | |
| | | gcctcccgaagttgcttgaaagcactcggagaattgtgca | |
| | | ggtgtcatttatctatgaccaataggaagagcaaccagtt | |
| | | actatgagtgaaagggagccagaagactgattggagggcc | |
| | | ctatcttgtgagtggggcatctgttggactttccacctgg | |
| | | tcatatactctgcagctgttagaatgtgcaagcacttggg | |
| | | gacagcatgagcttgctgttgtacacagggtatttctaga | |
| | | agcagaaatagactgggaagatgcacaaccaaggggttac | |
| | | aggcatcgcccatgctcctcacctgtattttgtaatcaga | |
| | | aataaattgcttttaaagaaaaaaaaaaaaaaaaaa | |
| B2M | NM_004048.2 | aatataagtggaggcgtcgcgctggcgggcattcctgaag | 28 |
| | | ctgacagcattcgggccgagatgtctcgctccgtggcctt | |
| | | agctgtgctcgcgctactctctctttctggcctggaggct | |
| | | atccagcgtactccaaagattcaggtttactcacgtcatc | |
| | | cagcagagaatggaaagtcaaatttcctgaattgctatgt | |
| | | gtctgggtttcatccatccgacattgaagttgacttactg | |
| | | aagaatggagagagaattgaaaaagtggagcattcagact | |
| | | tgtctttcagcaaggactggtctttctatctcttgtacta | |
| | | cactgaattcacccccactgaaaaagatgagtatgcctgc | |
| | | cgtgtgaaccatgtgactttgtcacagcccaagatagtta | |
| | | agtgggatcgagacatgtaagcagcatcatggaggtttga | |
| | | agatgccgcatttggattggatgaattccaaattctgctt | |
| | | gcttgctttttaatattgatatgcttatacacttacactt | |
| | | tatgcacaaaatgtagggttataataatgttaacatggac | |
| | | atgatcttctttataattctactttgagtgctgtctccat | |
| | | gtttgatgtatctgagcaggttgctccacaggtagctcta | |
| | | ggagggctggcaacttagaggtggggagcagagaattctc | |
| | | ttatccaacatcaacatcttggtcagatttgaactcttca | |
| | | atctcttgcactcaaagcttgttaagatagttaagcgtgc | |
| | | ataagttaacttccaatttacatactctgcttagaatttg | |
| | | ggggaaaatttagaaatataattgacaggattattggaaa | |
| | | tttgttataatgaatgaaacattttgtcatataagattca | |
| | | tatttacttcttatacatttgataaagtaaggcatggttg | |
| | | tggttaatctggtttatttttgttccacaagttaaataaa | |
| | | tcataaaacttgatgtgttatctctta | |
| YWHAZ | NM_003406.3 | ctttctccttccccttcttccgggctcccgtcccggctca | 29 |
| | | tcacccggcctgtggcccactcccaccgccagctggaacc | |
| | | ctggggactacgacgtccctcaaaccttgcttctaggaga | |
| | | taaaaagaacatccagtcatggataaaaatgagctggttc | |
| | | agaaggccaaactggccgagcaggctgagcgatatgatga | |
| | | catggcagcctgcatgaagtctgtaactgagcaaggagct | |
| | | gaattatccaatgaggagaggaatcttctctcagttgctt | |
| | | ataaaaatgttgtaggagcccgtaggtcatcttggagggt | |
| | | cgtctcaagtattgaacaaaagacggaaggtgctgagaaa | |
| | | aaacagcagatggctcgagaatacagagagaaaattgaga | |
| | | cggagctaagagatatctgcaatgatgtactgtctctttt | |
| | | ggaaaagttcttgatccccaatgcttcacaagcagagagc | |
| | | aaagtcttctatttgaaaatgaaaggagattactaccgtt | |
| | | acttggctgaggttgccgctggtgatgacaagaaagggat | |
| | | tgtcgatcagtcacaacaagcataccaagaagcttttgaa | |
| | | atcagcaaaaaggaaatgcaaccaacacatcctatcagac | |
| | | tgggtctggcccttaacttctctgtgttctattatgagat | |
| | | tctgaactccccagagaaagcctgctctcttgcaaagaca | |
| | | gcttttgatgaagccattgctgaacttgatacattaagtg | |
| | | aagagtcatacaaagacagcacgctaataatgcaattact | |
| | | gagagacaacttgacattgtggacatcggatacccaagga | |
| | | gacgaagctgaagcaggagaaggaggggaaaattaaccgg | |
| | | ccttccaacttttgtctgcctcattctaaaatttacacag | |
| | | tagaccatttgtcatccatgctgtcccacaaatagttttt | |
| | | tgtttacgatttatgacaggtttatgttacttctatttga | |
| | | atttctatatttcccatgtggtttttatgtttaatattag | |
| | | gggagtagagccagttaacatttagggagttatctgtttt | |
| | | catcttgaggtggccaatatggggatgtggaatttttata | |
| | | caagttataagtgtttggcatagtacttttggtacattgt | |
| | | ggcttcaaaagggccagtgtaaaactgcttccatgtctaa | |
| | | gcaaagaaaactgcctacatactggtttgtcctggcgggg | |
| | | aataaaagggatcattggttccagtcacaggtgtagtaat | |
| | | tgtgggtactttaaggtttggagcacttacaaggctgtgg | |
| | | tagaatcataccccatggataccacatattaaaccatgta | |
| | | tatctgtggaatactcaatgtgtacacctttgactacagc | |
| | | tgcagaagtgttcctttagacaaagttgtgacccatttta | |
| | | ctctggataagggcagaaacggttcacattccattatttg | |
| | | taaagttacctgctgttagctttcattatttttgctacac | |
| | | tcattttatttgtatttaaatgttttaggcaacctaagaa | |
| | | caaatgtaaaagtaaagatgcaggaaaaatgaattgcttg | |
| | | gtattcattacttcatgtatatcaagcacagcagtaaaac | |
| | | aaaaacccatgtatttaacttttttttaggatttttgctt | |
| | | ttgtgatttttttttttttgatacttgcctaacatgcatg | |
| | | tgctgtaaaaatagttaacagggaaataacttgagatgat | |
| | | ggctagctttgtttaatgtcttatgaaattttcatgaaca | |
| | | atccaagcataattgttaagaacacgtgtattaaattcat | |
| | | gtaagtggaataaaagttttatgaatggacttttcaacta | |
| | | ctttctctacagcttttcatgtaaattagtcttggttctg | |
| | | aaacttctctaaaggaaattgtacattttttgaaatttat | |
| | | tccttattccctcttggcagctaatgggctcttaccaagt | |
| | | ttaaacacaaaatttatcataacaaaaatactactaatat | |
| | | aactactgtttccatgtcccatgatcccctctcttcctcc | |
| | | ccaccctgaaaaaaatgagttcctattttttctgggagag | |
| | | ggggggattgattagaaaaaaatgtagtgtgttccattta | |
| | | aaattttggcatatggcattttctaacttaggaagccaca | |
| | | atgttcttggcccatcatgacattgggtagcattaactgt | |
| | | aagttttgtgcttccaaatcactttttggtttttaagaat | |
| | | ttcttgatactcttatagcctgccttcaattttgatcctt | |
| | | tattctttctatttgtcaggtgcacaagattaccttcctg | |
| | | ttttagccttctgtcttgtcaccaaccattcttacttggt | |
| | | ggccatgtacttggaaaaaggccgcatgatctttctggct | |
| | | ccactcagtgtctaaggcaccctgcttcctttgcttgcat | |
| | | cccacagactatttccctcatcctatttactgcagcaaat | |
| | | ctctccttagttgatgagactgtgtttatctccctttaaa | |
| | | accctacctatcctgaatggtctgtcattgtctgccttta | |
| | | aaatccttcctctttcttcctcctctattctctaaataat | |
| | | gatggggctaagttatacccaaagctcactttacaaaata | |
| | | tttcctcagtactttgcagaaaacaccaaacaaaaatgcc | |
| | | attttaaaaaaggtgtattttttcttttagaatgtaagct | |
| | | cctcaagagcagggacaatgttttctgtatgttctattgt | |
| | | gcctagtacactgtaaatgctcaataaatattgatgatgg | |
| | | gaggcagtgagtcttgatgataagggtgagaaactgaaat | |
| | | cccaaacactgttttgttgcttgttttattatgacctcag | |
| | | attaaattgggaaatattggcccttttgaataattgtccc | |
| | | aaatattacattcaaataaaagtgcaatggagaaaaaaaa | |
| | | aaa | |
| SDHA | NM_004168.3 | actgcagccccgctcgactccggcgtggtgcgcaggcgcg | 30 |
| | | gtatcccccctcccccgccagctcgaccccggtgtggtgc | |
| | | gcaggcgcagtctgcgcagggactggcgggactgcgcggc | |
| | | ggcaacagcagacatgtcgggggtccggggcctgtcgcgg | |
| | | ctgctgagcgctcggcgcctggcgctggccaaggcgtggc | |
| | | caacagtgttgcaaacaggaacccgaggttttcacttcac | |
| | | tgttgatgggaacaagagggcatctgctaaagtttcagat | |
| | | tccatttctgctcagtatccagtagtggatcatgaatttg | |
| | | atgcagtggtggtaggcgctggaggggcaggcttgcgagc | |
| | | tgcatttggcctttctgaggcagggtttaatacagcatgt | |
| | | gttaccaagctgtttcctaccaggtcacacactgttgcag | |
| | | cacagggaggaatcaatgctgctctggggaacatggagga | |
| | | ggacaactggaggtggcatttctacgacaccgtgaagggc | |
| | | tccgactggctgggggaccaggatgccatccactacatga | |
| | | cggagcaggcccccgccgccgtggtcgagctagaaaatta | |
| | | tggcatgccgtttagcagaactgaagatgggaagatttat | |
| | | cagcgtgcatttggtggacagagcctcaagtttggaaagg | |
| | | gcgggcaggcccatcggtgctgctgtgtggctgatcggac | |
| | | tggccactcgctattgcacaccttatatggaaggtctctg | |
| | | cgatatgataccagctattttgtggagtattttgccttgg | |
| | | atctcctgatggagaatggggagtgccgtggtgtcatcgc | |
| | | actgtgcatagaggacgggtccatccatcgcataagagca | |
| | | aagaacactgttgttgccacaggaggctacgggcgcacct | |
| | | acttcagctgcacgtctgcccacaccagcactggcgacgg | |
| | | cacggccatgatcaccagggcaggccttccttgccaggac | |
| | | ctagagtttgttcagttccaccctacaggcatatatggtg | |
| | | ctggttgtctcattacggaaggatgtcgtggagagggagg | |
| | | cattctcattaacagtcaaggcgaaaggtttatggagcga | |
| | | tacgcccctgtcgcgaaggacctggcgtctagagatgtgg | |
| | | tgtctcggtccatgactctggagatccgagaaggaagagg | |
| | | ctgtggccctgagaaagatcacgtctacctgcagctgcac | |
| | | cacctacctccagagcagctggccacgcgcctgcctggca | |
| | | tttcagagacagccatgatcttcgctggcgtggacgtcac | |
| | | gaaggagccgatccctgtcctccccaccgtgcattataac | |
| | | atgggcggcattcccaccaactacaaggggcaggtcctga | |
| | | ggcacgtgaatggccaggatcagattgtgcccggcctgta | |
| | | cgcctgtggggaggccgcctgtgcctcggtacatggtgcc | |
| | | aaccgcctcggggcaaactcgctcttggacctggttgtct | |
| | | ttggtcgggcatgtgccctgagcatcgaagagtcatgcag | |
| | | gcctggagataaagtccctccaattaaaccaaacgctggg | |
| | | gaagaatctgtcatgaatcttgacaaattgagatttgctg | |
| | | atggaagcataagaacatcggaactgcgactcagcatgca | |
| | | gaagtcaatgcaaaatcatgctgccgtgttccgtgtggga | |
| | | agcgtgttgcaagaaggttgtgggaaaatcagcaagctct | |
| | | atggagacctaaagcacctgaagacgttcgaccggggaat | |
| | | ggtctggaacacggacctggtggagaccctggagctgcag | |
| | | aacctgatgctgtgtgcgctgcagaccatctacggagcag | |
| | | aggcacggaaggagtcacggggcgcgcatgccagggaaga | |
| | | ctacaaggtgcggattgatgagtacgattactccaagccc | |
| | | atccaggggcaacagaagaagccctttgaggagcactgga | |
| | | ggaagcacaccctgtcctatgtggacgttggcactgggaa | |
| | | ggtcactctggaatatagacccgtgatcgacaaaactttg | |
| | | aacgaggctgactgtgccaccgtcccgccagccattcgct | |
| | | cctactgatgagacaagatgtggtgatgacagaatcagct | |
| | | tttgtaattatgtataatagctcatgcatgtgtccatgtc | |
| | | ataactgtcttcatacgcttctgcactctggggaagaagg | |
| | | agtacattgaagggagattggcacctagtggctgggagct | |
| | | tgccaggaacccagtggccagggagcgtggcacttacctt | |
| | | tgtcccttgcttcattcttgtgagatgataaaactgggca | |
| | | cagctcttaaataaaatataaatgaacaaactttctttta | |
| | | tttccaaatccatttgaaatattttactgttgtgacttta | |
| | | gtcatatttgttgacctaaaaatcaaatgtaatctttgta | |
| | | ttgtgttacatcaaaatccagatattttgtatagtttctt | |
| | | ttttctttttcttttcttttttttttttgagacaggatcg | |
| | | gtgcagtagtacaatcacagctcactgcagcctcaaactc | |
| | | ctgggcagctcaggtgatcttcctgactcagccttctgag | |
| | | tagttggggctacaggtgtgcaccaccatgcccagctcat | |
| | | ttattttgtaattgtagggacagggtctcactgtgttgcc | |
| | | taggctggtctcaagtgatcctccctccttggcctcccaa | |
| | | ggtgctggaattataggtgtgaacaaaccaaaaaaaaaaa | |
| | | aaa | |
| HPRT1 | NM_000194.2 | ggcggggcctgcttctcctcagcttcaggcggctgcgacg | 31 |
| | | agccctcaggcgaacctctcggctttcccgcgcggcgccg | |
| | | cctcttgctgcgcctccgcctcctcctctgctccgccacc | |
| | | ggcttcctcctcctgagcagtcagcccgcgcgccggccgg | |
| | | ctccgttatggcgacccgcagccctggcgtcgtgattagt | |
| | | gatgatgaaccaggttatgaccttgatttattttgcatac | |
| | | ctaatcattatgctgaggatttggaaagggtgtttattcc | |
| | | tcatggactaattatggacaggactgaacgtcttgctcga | |
| | | gatgtgatgaaggagatgggaggccatcacattgtagccc | |
| | | tctgtgtgctcaaggggggctataaattctttgctgacct | |
| | | gctggattacatcaaagcactgaatagaaatagtgataga | |
| | | tccattcctatgactgtagattttatcagactgaagagct | |
| | | attgtaatgaccagtcaacaggggacataaaagtaattgg | |
| | | tggagatgatctctcaactttaactggaaagaatgtcttg | |
| | | attgtggaagatataattgacactggcaaaacaatgcaga | |
| | | ctttgctttccttggtcaggcagtataatccaaagatggt | |
| | | caaggtcgcaagcttgctggtgaaaaggaccccacgaagt | |
| | | gttggatataagccagactttgttggatttgaaattccag | |
| | | acaagtttgttgtaggatatgcccttgactataatgaata | |
| | | cttcagggatttgaatcatgtttgtgtcattagtgaaact | |
| | | ggaaaagcaaaatacaaagcctaagatgagagttcaagtt | |
| | | gagtttggaaacatctggagtcctattgacatcgccagta | |
| | | aaattatcaatgttctagttctgtggccatctgcttagta | |
| | | gagctttttgcatgtatcttctaagaattttatctgtttt | |
| | | gtactttagaaatgtcagttgctgcattcctaaactgttt | |
| | | atttgcactatgagcctatagactatcagttccctttggg | |
| | | cggattgttgtttaacttgtaaatgaaaaaattctcttaa | |
| | | accacagcactattgagtgaaacattgaactcatatctgt | |
| | | aagaaataaagagaagatatattagttttttaattggtat | |
| | | tttaatttttatatatgcaggaaagaatagaagtgattga | |
| | | atattgttaattataccaccgtgtgttagaaaagtaagaa | |
| | | gcagtcaattttcacatcaaagacagcatctaagaagttt | |
| | | tgttctgtcctggaattattttagtagtgtttcagtaatg | |
| | | ttgactgtattttccaacttgttcaaattattaccagtga | |
| | | atctttgtcagcagttcccttttaaatgcaaatcaataaa | |
| | | ttcccaaaaatttaaaaaaaaaaaaaaaaaaaaaa | |

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the terms “polynucleotide” and “nucleic acid molecule” are used interchangeably to mean a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In other aspects, a nucleic acid molecule or nucleic acid sequence comprises other kinds of nucleic acid structures such a for instance a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees centigrade and temperatures for washing in 0.1×SSC/0.1% SDS are above 55 degrees C., and preferably to stringent hybridization conditions.

As used herein, the term “normalization” or “normalizer” refers to the expression of a differential value in terms of a standard value to adjust for effects which arise from technical variation due to sample handling, sample preparation, and measurement methods rather than biological variation of biomarker concentration in a sample. For example, when measuring the expression of a differentially expressed protein, the absolute value for the expression of the protein can be expressed in terms of an absolute value for the expression of a standard protein that is substantially constant in expression.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarkers. Examples of biological samples include tissue, organs, or bodily fluids such as whole blood, plasma, serum, tissue, lavage or any other specimen used for detection of disease.

The term “subject” as used herein refers to a mammal, preferably a human. In some aspects, a subject can have at least one colon cancer symptom. In some aspects, a subject can have a predisposition or familial history for developing a colon cancer. A subject can also have been previously diagnosed with a colon cancer and is tested for cancer recurrence.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold, 2-fold, 3-fold, 4-fold, 5-fold or more, or any values in between 1-fold and five-fold. The change may be statistically significant with a p value of 0.1, 0.05, 0.001, or 0.0001.

The term “stable disease” refers to a diagnosis for the presence of a colon cancer,

however the colon cancer has been treated and remains in a stable condition, i.e. one that that is not progressive, as determined by imaging data and/or best clinical judgment.

The term “progressive disease” refers to a diagnosis for the presence of a highly active state of a colon cancer, i.e. one has not been treated and is not stable or has been treated and has not responded to therapy, or has been treated and active disease remains, as determined by imaging data and/or best clinical judgment.

The term “neoplastic disease” refers to any abnormal growth of cells or tissues being either benign (non-cancerous) or malignant (cancerous). For example, the neoplastic disease can be a colon cancer.

The term “neoplastic tissue” refers to a mass of cells that grow abnormally.

The term “non-neoplastic tissue” refers to a mass of cells that grow normally.

The term “immunotherapy” can refer to activating immunotherapy or suppressing immunotherapy. As will be appreciated by those in the art, activating immunotherapy refers to the use of a therapeutic agent that induces, enhances, or promotes an immune response, including, e.g., a T cell response while suppressing immunotherapy refers to the use of a therapeutic agent that interferes with, suppresses, or inhibits an immune response, including, e.g., a T cell response. Activating immunotherapy may comprise the use of checkpoint inhibitors. Activating immunotherapy may comprise administering to a subject a therapeutic agent that activates a stimulatory checkpoint molecule. Stimulatory checkpoint molecules include, but are not limited to, CD27, CD28, CD40, CD122, CD137, OX40, GITR and ICOS. Therapeutic agents that activate a stimulatory checkpoint molecule include, but are not limited to, MEDI0562, TGN1412, CDX-1127, lipocalin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 108 M or less, e.g. from 108 M to 1013 M, e.g., from 109 M to 1013 M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

Administering chemotherapy to a subject can comprise administering a therapeutically effective dose of at least one chemotherapeutic agent. Chemotherapeutic agents include, but are not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abemaciclib, Abiraterone acetate, Abraxane, Accutane, Actinomycin-D, Adcetris, Ado-Trastuzumab Emtansine, Adriamycin, Adrucil, Afatinib, Afinitor, Agrylin, Ala-Cort, Aldesleukin, Alemtuzumab, Alecensa, Alectinib, Alimta, Alitretinoin, Alkaban-AQ, Alkeran, All-transretinoic Acid, Alpha Interferon, Altretamine, Alunbrig, Amethopterin, Amifostine, Aminoglutethimide, Anagrelide, Anandron, Anastrozole, Apalutamide, Arabinosylcytosine, Ara-C, Aranesp, Aredia, Arimidex, Aromasin, Arranon, Arsenic Trioxide, Arzerra, Asparaginase, Atezolizumab, Atra, Avastin, Avelumab, Axicabtagene Ciloleucel, Axitinib, Azacitidine, Bavencio, Bcg, Beleodaq, Belinostat, Bendamustine, Bendeka, Besponsa, Bevacizumab, Bexarotene, Bexxar, Bicalutamide, Bicnu, Blenoxane, Bleomycin, Blinatumomab, Blincyto, Bortezomib, Bosulif, Bosutinib, Brentuximab Vedotin, Brigatinib, Busulfan, Busulfex, C225, Cabazitaxel, Cabozantinib, Calcium Leucovorin, Campath, Camptosar, Camptothecin-11, Capecitabine, Caprelsa, Carac, Carboplatin, Carfilzomib, Carmustine, Carmustine Wafer, Casodex, CCI-779, Ccnu, Cddp, Ceenu, Ceritinib, Cerubidine, Cetuximab, Chlorambucil, Cisplatin, Citrovorum Factor, Cladribine, Clofarabine, Clolar, Cobimetinib, Cometriq, Cortisone, Cosmegen, Cotellic, Cpt-11, Crizotinib, Cyclophosphamide, Cyramza, Cytadren, Cytarabine, Cytarabine Liposomal, Cytosar-U, Cytoxan, Dabrafenib, Dacarbazine, Dacogen, Dactinomycin, Daratumumab, Darbepoetin Alfa, Darzalex, Dasatinib, Daunomycin, Daunorubicin, Daunorubicin Cytarabine (Liposomal), daunorubicin-hydrochloride, Daunorubicin Liposomal, DaunoXome, Decadron, Decitabine, Degarelix, Delta-Cortef, Deltasone, Denileukin Diftitox, Denosumab, DepoCyt, Dexamethasone, Dexamethasone Acetate, Dexamethasone Sodium Phosphate, Dexasone, Dexrazoxane, Dhad, Dic, Diodex, Docetaxel, Doxil, Doxorubicin, Doxorubicin Liposomal, Droxia, DTIC, Dtic-Dome, Duralone, Durvalumab, Eculizumab, Efudex, Ellence, Elotuzumab, Eloxatin, Elspar, Eltrombopag, Emcyt, Empliciti, Enasidenib, Enzalutamide, Epirubicin, Epoetin Alfa, Erbitux, Eribulin, Erivedge, Erleada, Erlotinib, Erwinia L-asparaginase, Estramustine, Ethyol, Etopophos, Etoposide, Etoposide Phosphate, Eulexin, Everolimus, Evista, Exemestane, Fareston, Farydak, Faslodex, Femara, Filgrastim, Firmagon, Floxuridine, Fludara, Fludarabine, Fluoroplex, Fluorouracil, Fluorouracil (cream), Fluoxymesterone, Flutamide, Folinic Acid, Folotyn, Fudr, Fulvestrant, G-Csf, Gazyva, Gefitinib, Gemcitabine, Gemtuzumab ozogamicin, Gemzar, Gilotrif, Gleevec, Gleostine, Gliadel Wafer, Gm-Csf, Goserelin, Granix, Granulocyte-Colony Stimulating Factor, Granulocyte Macrophage Colony Stimulating Factor, Halaven, Halotestin, Herceptin, Hexadrol, Hexalen, Hexamethylmelamine, Hmm, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibrance, Ibritumomab, Ibritumomab Tiuxetan, Ibrutinib, Iclusig, Idamycin, Idarubicin, Idelalisib, Idhifa, Ifex, IFN-alpha, Ifosfamide, IL-11, IL-2, Imbruvica, Imatinib Mesylate, Imfinzi, Imidazole Carboxamide, Imlygic, Inlyta, Inotuzumab Ozogamicin, Interferon-Alfa, Interferon Alfa-2b (PEG Conjugate), Interleukin-2, Interleukin-11, Intron A (interferon alfa-2b), Ipilimumab, Iressa, Irinotecan, Irinotecan (Liposomal), Isotretinoin, Istodax, Ixabepilone, Ixazomib, Ixempra, Jakafi, Jevtana, Kadcyla, Keytruda, Kidrolase, Kisqali, Kymriah, Kyprolis, Lanacort, Lanreotide, Lapatinib, Lartruvo, L-Asparaginase, Lbrance, Lcr, Lenalidomide, Lenvatinib, Lenvima, Letrozole, Leucovorin, Leukeran, Leukine, Leuprolide, Leurocristine, Leustatin, Liposomal Ara-C, Liquid Pred, Lomustine, Lonsurf, L-PAM, L-Sarcolysin, Lupron, Lupron Depot, Lynparza, Marqibo, Matulane, Maxidex, Mechlorethamine, Mechlorethamine Hydrochloride, Medralone, Medrol, Megace, Megestrol, Megestrol Acetate, Mekinist, Mercaptopurine, Mesna, Mesnex, Methotrexate, Methotrexate Sodium, Methylprednisolone, Meticorten, Midostaurin, Mitomycin, Mitomycin-C, Mitoxantrone, M-Prednisol, MTC, MTX, Mustargen, Mustine, Mutamycin, Myleran, Mylocel, Mylotarg, Navelbine, Necitumumab, Nelarabine, Neosar, Neratinib, Nerlynx, Neulasta, Neumega, Neupogen, Nexavar, Nilandron, Nilotinib, Nilutamide, Ninlaro, Nipent, Niraparib, Nitrogen Mustard, Nivolumab, Nolvadex, Novantrone, Nplate, Obinutuzumab, Octreotide, Octreotide Acetate, Odomzo, Ofatumumab, Olaparib, Olaratumab, Omacetaxine, Oncospar, Oncovin, Onivyde, Ontak, Onxal, Opdivo, Oprelvekin, Orapred, Orasone, Osimertinib, Otrexup, Oxaliplatin, Paclitaxel, Paclitaxel Protein-bound, Palbociclib, Pamidronate, Panitumumab, Panobinostat, Panretin, Paraplatin, Pazopanib, Pediapred, Peg Interferon, Pegaspargase, Pegfilgrastim, Peg-Intron, PEG-L-asparaginase, Pembrolizumab, Pemetrexed, Pentostatin, Perjeta, Pertuzumab, Phenylalanine Mustard, Platinol, Platinol-AQ, Pomalidomide, Pomalyst, Ponatinib, Portrazza, Pralatrexate, Prednisolone, Prednisone, Prelone, Procarbazine, Procrit, Proleukin, Prolia, Prolifeprospan 20 with Carmustine Implant, Promacta, Provenge, Purinethol, Radium 223 Dichloride, Raloxifene, Ramucirumab, Rasuvo, Regorafenib, Revlimid, Rheumatrex, Ribociclib, Rituxan, Rituxan Hycela, Rituximab, Rituximab Hyalurodinase, Roferon-A (Interferon Alfa-2a), Romidepsin, Romiplostim, Rubex, Rubidomycin Hydrochloride, Rubraca, Rucaparib, Ruxolitinib, Rydapt, Sandostatin, Sandostatin LAR, Sargramostim, Siltuximab, Sipuleucel-T, Soliris, Solu-Cortef, Solu-Medrol, Somatuline, Sonidegib, Sorafenib, Sprycel, Sti-571, Stivarga, Streptozocin, SU11248, Sunitinib, Sutent, Sylvant, Synribo, Tafinlar, Tagrisso, Talimogene Laherparepvec, Tamoxifen, Tarceva, Targretin, Tasigna, Taxol, Taxotere, Tecentriq, Temodar, Temozolomide, Temsirolimus, Teniposide, Tespa, Thalidomide, Thalomid, TheraCys, Thioguanine, Thioguanine Tabloid, Thiophosphoamide, Thioplex, Thiotepa, Tice, Tisagenlecleucel, Toposar, Topotecan, Toremifene, Torisel, Tositumomab, Trabectedin, Trametinib, Trastuzumab, Treanda, Trelstar, Tretinoin, Trexall, Trifluridine/Tipiricil, Triptorelin pamoate, Trisenox, Tspa, T-VEC, Tykerb, Valrubicin, Valstar, Vandetanib, VCR, Vectibix, Velban, Velcade, Vemurafenib, Venclexta, Venetoclax, VePesid, Verzenio, Vesanoid, Viadur, Vidaza, Vinblastine, Vinblastine Sulfate, Vincasar Pfs, Vincristine, Vincristine Liposomal, Vinorelbine, Vinorelbine Tartrate, Vismodegib, Vlb, VM-26, Vorinostat, Votrient, VP-16, Vumon, Vyxeos, Xalkori Capsules, Xeloda, Xgeva, Xofigo, Xtandi, Yervoy, Yescarta, Yondelis, Zaltrap, Zanosar, Zarxio, Zejula, Zelboraf, Zevalin, Zinecard, Ziv-aflibercept, Zoladex, Zoledronic Acid, Zolinza, Zometa, Zydelig, Zykadia, Zytiga, or any combination thereof.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### EXAMPLES

The disclosure is further illustrated by the following examples, which are not to be construed as limiting this disclosure in scope or spirit to the specific procedures herein described. It is to be understood that the examples are provided to illustrate certain aspects and that no limitation to the scope of the disclosure is intended thereby. It is to be further understood that resort may be had to various other aspects, embodiments, modifications, and equivalents thereof which may suggest themselves to those skilled in the art without departing from the spirit of the present disclosure and/or scope of the appended claims.

### Example 1. Derivation of a 13-Marker Gene Panel

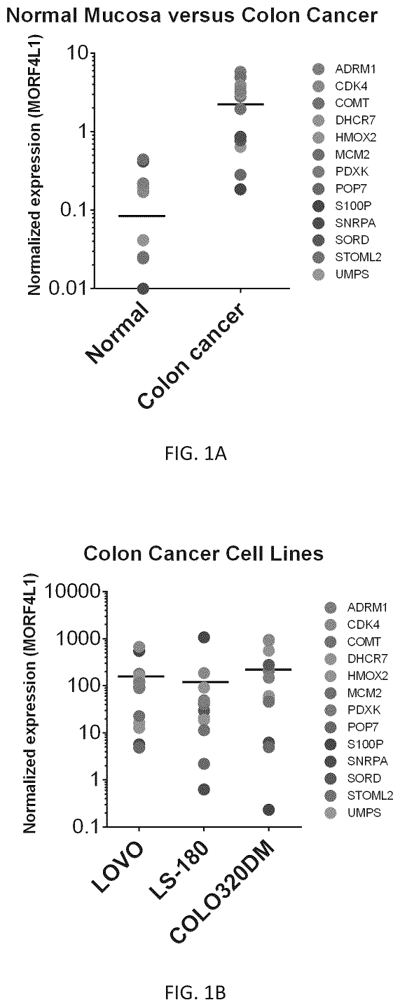

Raw probe intensities from n=24 colon cancer tumor tissue samples were compared to n =22 control colon mucosa to identify genes that best discriminated between disease using the transcriptional profile of E-MTAB-57. Gene co-expression networks were generated to identify temporal patterns of gene regulation associated with colon cancer. A total of 513 nodes with 53,786 links were identified. Differential expression analysis identified 103 genes were upregulated in tumor tissue compared to blood. To identify blood-specific colon cancer gene biomarkers, we evaluated expression of the 103 genes in peripheral blood transcriptomes (n=7). Thirty-three (32%) of the 103 genes were below the level of detection in blood identifying these as candidate genes. Evaluation of transcripts in a preliminary dataset of blood samples from colon cancer (n=20) and matched normal blood (n=20) identified thirteen genes and one house-keeping gene as markers of colon cancer (Table 2). These genes were demonstrated to be highly expressed in colon cancer tumor tissue compared to normal mucosa and in three different colon cancer cell lines, LOVO (metastatic, hyperdiploid, MSI unstable cell line), LS-180 (derived from a Duke's B, colorectal adenocarcinoma) and Colo 320DM (derived from a Duke's C, colorectal adenocarcinoma). These data demonstrate target transcripts are produced by neoplastically transformed colon mucosal cells (FIGS. 1A-1B).

An artificial intelligence model of colon cancer disease was built using normalized gene expression of these 13 markers in whole blood from Controls (n=120) and Colon Cancers (n=272) samples. The dataset was randomly split into training and testing partitions for model creation and validation respectively. Twelve algorithms were evaluated (XGB, RF, glmnet, cforest, CART, treebag, knn, nnet, SVM-radial, SVM-linear, NB and mlp). The top performing algorithm (XGB—- “gradient boosting”) best predicted the training data. In the test set, XGB produced probability scores that predicted the sample. Each probability score reflects the “certainty” of an algorithm that an unknown sample belongs to either “Control” or “Colon Cancer” class. For example, an unknown sample S1 can have the following probability vector [Control=20%, Colon Cancer=80%]. This sample would be considered a colon cancer sample.

### Example 2. Clinical Utility

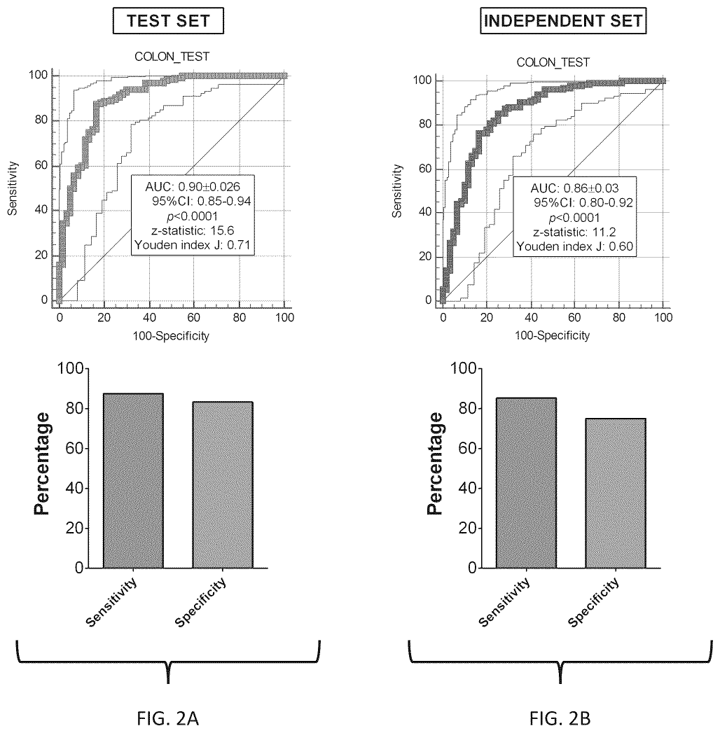

The data (receiver operator cuver analysis and metrics) for the utility of the test to differentiate patients with colon cancer (n=136) from controls (n=60) in the training and test sets are included in FIGS. 2A-2B. The score exhibited an area under the curve (AUC) of 0.90 (training) and 0.86 (test set). The metrics are: sensitivity: 85.3-87.5% and specificity: 75-83.3%.

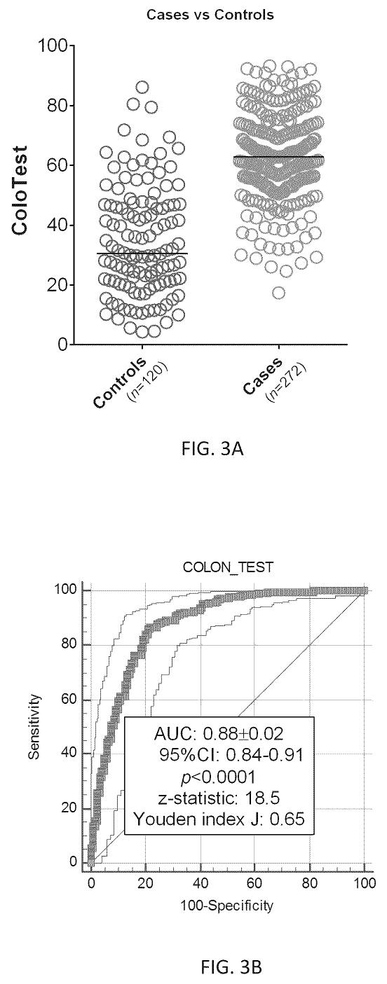

Overall, ColoTest scores were significantly elevated in cancers (63±1%) and controls (34±2%) (FIGS. 3A-3B). The overall accuracy (training and test cohort) is 84%, with an AUC: 0.88. The z-statistic for differentiating controls was 18.5.

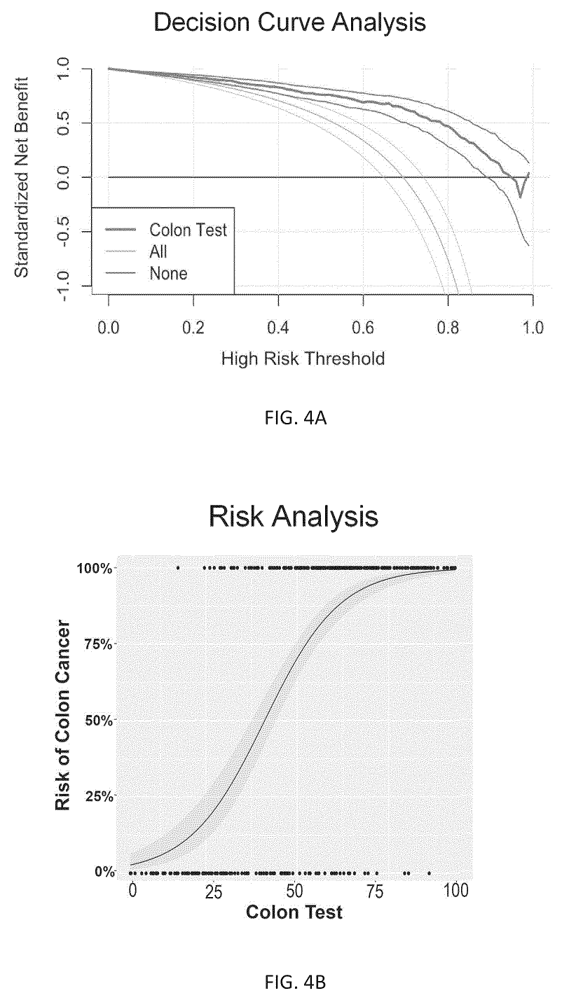

A decision curve analysis was used to quantify the clinical benefit of the diagnostic test (FIGS. 4A-4B). The ColoTest exhibited >50% standardized predictive benefit up to a risk threshold of 80%. The probit risk assessment plot identified a ColoTest score >50% was 75% accurate for predicting colon cancer in a blood sample. This was increased to >80% at a ColoTest score ≥60%. The tool can therefore accurately differentiate between controls and colon cancer disease.

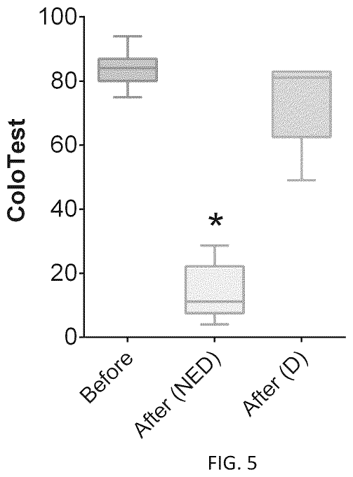

Specific evaluation of a colon cancer cohort before and after surgery identified that complete removal of a tumor and no evidence of disease was associated with a significant decrease (p<0.0001) in the ColoTest (FIG. 5). Levels were not significantly different in those with evidence of residual disease.

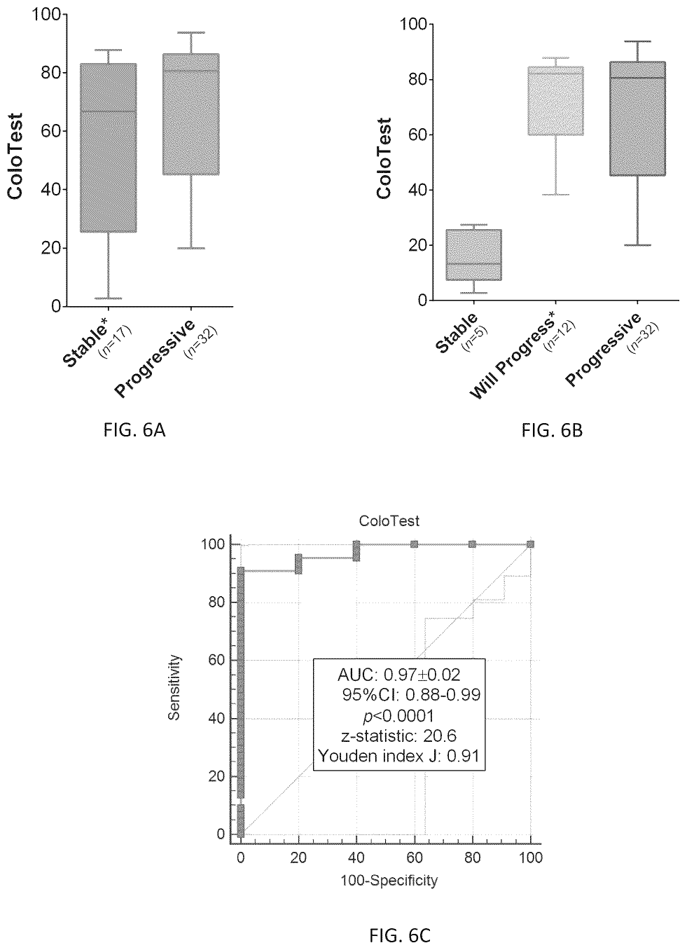

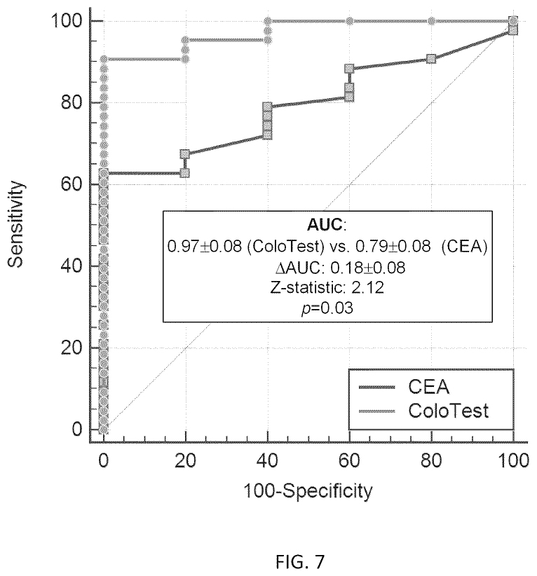

Examination of a separate colon cancer cohort by disease status (clinical evaluation at time of blood-draw) identified that the ColoTest was not significantly different between stable (n=17: 56±7%) and progressive disease (n=32: 68±4%) (FIGS. 6A-6C). However, 12 of the 17 patients progressed with 3 months of blood collection. Those that did progress exhibited elevated ColoTest scores at time of blood draw (n=12: 73±4%) that were not different to those with progressive disease at time of blood draw (n=32: 68±4%) (FIGS. 6A-6C). Levels in patients with stable disease were significantly lower (n=5: 16±4%, p<0.0001). A direct comparison between the ColoTest and CEA in these samples identified that the gene expression assay was significantly more sensitive (p<0.05) than CEA for predicting disease progression (FIG. 7). The ColoTest tool can therefore accurately predict progressive colon cancer disease.

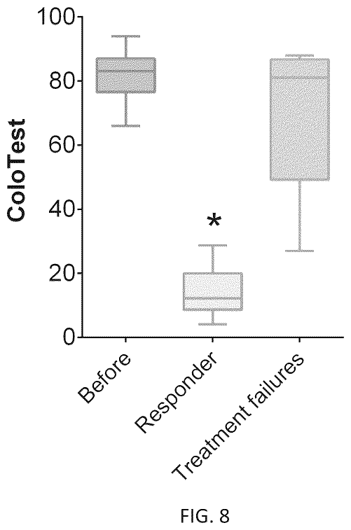

ROC analysis identified the ColoTest had an AUC: 0.97 for differentiating stable from progressive disease. The z-statistic for differentiating controls was 20.6. Further evaluation of this cohort identified that patients who exhibited disease progression despite therapy exhibited higher scores than those responding to therapy (FIG. 8). Therapies included bevacizumab, chemotherapy and EGFR TKI inhibitors. The tool can therefore accurately identify treatment failure in colon cancer disease.

TABLE 2

| Colon Cancer Biomarker or | | | | | | | |
|---|---|---|---|---|---|---|---|
| Housekeeping Genes | | NCBI Chromosome | | | Amplicon | Exon | Assay |
| Symbol | Name | location | UniGene ID | RefSeq | length | Boundary | Location |
| ADRM1 | adhesion regulating | Chr.20: 62302056- | Hs.90107 | NM_007002.3 | 60 | 3-4 | 486 |
| | molecule 1 | 62308862 | | | | | |
| CDK4 | cyclin dependent | Chr.12: 57747727- | Hs.95577 | NM_000075.3 | 65 | 5-6 | 928 |
| | kinase 4 | 57752447 | | | | | |
| COMT | catechol-O- | Chr.22: 19941740- | Hs.370408 | NM_000754.3 | 118 | 5-6 | 864 |
| | methyltransferase | 19969975 | | | | | |
| DHCR7 | 7-dehydrocholesterol | Chr.11: 71434411- | Hs.503134 | NM_001163817.1 | 74 | 3-4 | 351 |
| | reductase | 71448431 | | | | | |
| HMOX2 | heme oxygenase 2 | Chr.16: 4474697- | Hs.284279 | NM_001127204.1 | 81 | 5-6 | 1002 |
| | | 4510347 | | | | | |
| MCM2 | minichromosome | Chr.3: 127598357- | Hs.477481 | NM_004526.3 | 67 | 13-14 | 2374 |
| | maintenance complex | 127622436 | | | | | |
| | component 2 | | | | | | |
| MORF4L1 | mortality factor 4 | Chr.15: 78872781- | Hs.374503 | NM_001265603.1 | 62 | 1 | 116 |
| (housekeeping | like 1 | 78897739 | | | | | |
| gene) | | | | | | | |
| PDXK | pyridoxal (pyridoxine, | Chr.21: 43719097- | Hs.284491 | NM_003681.4 | 103 | 9-10 | 959 |
| | vitamin B6) kinase | 43762307 | | | | | |
| POP7 | POP7 homolog, | Chr.7: 100706053- | Hs.416994 | NM_005837.2 | 136 | 2 | 828 |
| | ribonuclease P/MRP | 100707500 | | | | | |
| | subunit | | | | | | |
| S100P | S100 calcium binding | Chr.4: 6693839- | Hs.2962 | NM_005980.2 | 73 | 1-2 | 234 |
| | protein P | 6697170 | | | | | |
| SNRPA | small nuclear | Chr.19: 40750854- | Hs.466775 | NM_004596.4 | 123 | 3-4 | 986 |
| | ribonucleoprotein | 40765392 | | | | | |
| | polypeptide A | | | | | | |
| SORD | sorbitol | Chr.15: 45023104- | Hs.878 | NM_003104.5 | 72 | 4-5 | 601 |
| | dehydrogenase | 45075089 | | | | | |
| STOML2 | stomatin like 2 | Chr.9: 35099776- | Hs.3439 | NM_001287031.1 | 68 | 2-3 | 290 |
| | | 35103195 | | | | | |
| UMPS | uridine | Chr.3: 124730366- | Hs.2057 | NM_000373.3 | 85 | 3-4 | 1082 |
| | monophosphate | 124749273 | | | | | |
| | synthetase | | | | | | |

### References

1. Siegel R L, Miller K D, Jemal A. Cancer Statistics, 2017. CA Cancer J Clin. 2017; 67: 7-30. doi: 10.3322/caac.21387. Epub 2017 Jan. 5.
2. Ferlay J, Steliarova-Foucher E, Lortet-Tieulent J, Rosso S, Coebergh J W, Comber H, Forman D, Bray F. Cancer incidence and mortality patterns in Europe: estimates for 40 countries in 2012. Eur J Cancer. 2013; 49: 1374-403.
3. Fritzmann J, Morkel M, Besser D, Budczies J, Kosel F, Brembeck F H, Stein U, Fichtner I, Schlag P M, Birchmeier W. A colorectal cancer expression profile that includes transforming growth factor beta inhibitor BAMBI predicts metastatic potential. Gastroenterology. 2009; 137: 165-75.
4. Chen V W, Hsieh M C, Charlton M E, Ruiz B A, Karlitz J, Altekruse S F, Ries L A, Jessup J M. Analysis of stage and clinical/prognostic factors for colon and rectal cancer from SEER registries: AJCC and collaborative stage data collection system. Cancer. 2014; 120: 3793-806.
5. Heald R J, Lockhart-Mummery H E. The lesion of the second cancer of the large bowel. Br J Surg. 1972; 59: 16-9.
6. Mokhles S, Macbeth F, Farewell V, Fiorentino F, Williams N R, Younes R N, Takkenberg J J, Treasure T. Meta-analysis of colorectal cancer follow-up after potentially curative resection. Br J Surg. 2016; 103: 1259-68.
7. Thomas S N, Zhu F, Schnaar R L, Alves C S, Konstantopoulos K. Carcinoembryonic antigen and CD44 variant isoforms cooperate to mediate colon carcinoma cell adhesion to E- and L-selectin in shear flow. J Biol Chem. 2008; 283: 15647-55.
8. Amri R, Bordeianou L G, Sylla P, Berger D L. Preoperative carcinoembryonic antigen as an outcome predictor in colon cancer. J Surg Oncol. 2013; 108: 14-8.
9. Jansen N, Coy J F. Diagnostic use of epitope detection in monocytes blood test for early detection of colon cancer metastasis. Future Oncol. 2013; 9: 605-9.
10. Locker G Y, Hamilton S, Harris J, Jessup J M, Kemeny N, Macdonald J S, Somerfield M R, Hayes D F, Bast R C, Jr. ASCO 2006 update of recommendations for the use of tumor markers in gastrointestinal cancer. J Clin Oncol. 2006; 24: 5313-27.
11. Warren J D, Xiong W, Bunker A M, Vaughn C P, Furtado L V, Roberts W L, Fang J C, Samowitz W S, Heichman K A. Septin 9 methylated DNA is a sensitive and specific blood test for colorectal cancer. BMC Med. 2011; 9: 133.: 10.1186/741-7015-9-133.
12. Mead R, Duku M, Bhandari P, Cree I A. Circulating tumour markers can define patients with normal colons, benign polyps, and cancers. Br J Cancer. 2011; 105: 239-45.
13. Molnar B, Floro L, Sipos F, Toth B, Sreter L, Tulassay Z. Elevation in peripheral blood circulating tumor cell number correlates with macroscopic progression in UICC stage IV colorectal cancer patients. Dis Markers. 2008; 24: 141-50. doi:
14. Mishaeli M, Klein B, Sadikov E, Bayer I, Koren R, Gal R, Rakowsky E, Levin I, Kfir B, Schachter J, Klein T. Initial TPS serum level as an indicator of relapse and survival in colorectal cancer. Anticancer Res. 1998; 18: 2101-5.
15. Piepoli A, Cotugno R, Merla G, Gentile A, Augello B, Quitadamo M, Merla A, Panza A, Carella M, Maglietta R, D'Addabbo A, Ancona N, Fusilli S, et al. Promoter methylation correlates with reduced NDRG2 expression in advanced colon tumour. BMC Med Genomics. 2009; 2: 11.: 10.1186/755-8794-2-11.

### Equivalents

While the present invention has been described in conjunction with the specific aspects set forth above, many alternatives, modifications and other variations thereof will be apparent to those of ordinary skill in the art. All such alternatives, modifications and variations are intended to fall within the spirit and scope of the present invention.

What is claimed is:

1. A method for detecting a colon cancer in a subject in need thereof, comprising:
determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene;
normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS;
inputting each normalized expression level into an algorithm to generate a score;
comparing the score with a predetermined cutoff value; and
producing a report, wherein the report identifies the presence of a colon cancer in the subject when the score is equal to or greater than the predetermined cutoff value or identifies the absence of a colon cancer in the subject when the score is less than the predetermined cutoff value.

2. A method for determining whether a colon cancer in a subject is stable or progressive, the method comprising:
determining the expression level of at least 14 biomarkers from a test sample from the subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene;
normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS;
inputting each normalized expression level into an algorithm to generate a score;
comparing the score with a predetermined cutoff value; and
producing a report, wherein the report identifies that the colon cancer is progressive when the score is equal to or greater than the predetermined cutoff value or identifies that the colon cancer is stable when the score is less than the predetermined cutoff value.

3. A method for determining the completeness of surgery in a subject having a colon cancer, the method comprising:
determining the expression level of at least 14 biomarkers from a test sample from the subject after the surgery by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene;
normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS;
inputting each normalized expression level into an algorithm to generate a score;
comparing the score with a predetermined cutoff value; and
producing a report, wherein the report identifies that the colon cancer is not completely removed when the score is equal to or greater than the predetermined cutoff value or identifies that the colon cancer is completely removed when the score is less than the predetermined cutoff value.

4. A method for evaluating the response of a subject having a colon cancer to a first therapy, the method comprising:
(1) at a first time point:
(a) determining the expression level of at least 14 biomarkers from a first test sample from the subject by contacting the first test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene;
(b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS;
(c) inputting each normalized expression level into an algorithm to generate a first score;
(2) at a second time point, wherein the second time point is after the first time point and after the administration of the therapy to the subject:
(a) determining the expression level of at least 14 biomarkers from a second test sample from the subject by contacting the second test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and the housekeeping gene,
(b) normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS;
(c) inputting each normalized expression level into the algorithm to generate a second score;
(3) comparing the first score with the second score; and
(4) producing a report, wherein the report identifies that the subject is responsive to the first therapy when the second score is significantly decreased as compared to the first score or identifies that the subject is not responsive to the first therapy when the second score is not significantly decreased as compared to the first score.

5. A method comprising:
determining the expression level of at least 14 biomarkers from a test sample from a subject by contacting the test sample with a plurality of agents specific to detect the expression of the at least 14 biomarkers, wherein the 14 biomarkers comprise ADRM1, CDK4, COMT, DHCR7, HMOX2, MCM2, PDXK, POP7, S100P, SNRPA, SORD, STOML2, UMPS, and a housekeeping gene;
normalizing the expression level of each of ADRM1, CDK4, COMT, DHCR7, HMOX2, MCM2, PDXK, POP7, S100P, SNRPA, SORD, STOML2, and UMPS to the expression level of the housekeeping gene, thereby obtaining a normalized expression level of each of ADRM1, CDK4, COMT, DHCR7, HMOX2, MCM2, PDXK, POP7, S100P, SNRPA, SORD, STOML2, and UMPS;
inputting each normalized expression level into an algorithm to generate a score;
comparing the score with a predetermined cutoff value; and
administering a first therapy to the subject when the score is equal to or greater than the predetermined cutoff value.

6. The method of claim 1, wherein the predetermined cutoff value is at least 50% on a scale of 0-100%.

7. The method of claim 1, wherein the predetermined cutoff value is at least 60% on a scale of 0-100%.

8. The method of claim 1, wherein the housekeeping gene is selected from the group consisting of MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, and HPRT1.

9. The method of claim 8, wherein the housekeeping gene is MORF4L1.

10. The method of claim 1, having a sensitivity greater than 85%.

11. The method of claim 1, having a specificity greater than 85%.

12. The method of claim 1, wherein at least one of the at least 14 biomarkers is RNA, cDNA or protein.

13. The method of claim 12, wherein when the biomarker is RNA, the RNA is reverse transcribed to produce cDNA, and the produced cDNA expression level is detected.

14. The method of claim 12, wherein the expression level of the biomarker is detected by forming a complex between the biomarker and a labeled probe or primer.

15. The method of claim 1, wherein the predetermined cutoff value is derived from a plurality of reference samples obtained from subjects not having or not diagnosed with a neoplastic disease.

16. The method of claim 15, wherein the neoplastic disease is colon cancer.

17. The method of claim 1, wherein the algorithm is XGBoost (XGB), Random Forest (RF), glmnet, cforest, Classification and Regression Trees for Machine Learning (CART), treebag, K-Nearest Neighbors (kNN), neural network (nnet), Support Vector Machine radial (SVM-radial), Support Vector Machine linear (SVM-linear), Naïve Bayes (NB), or multilayer perceptron (mlp).

18. The method of claim 17, wherein the algorithm is XGBoost.

19. The method of claim 1, further comprising administering to the subject a first therapy when the score is equal to or greater than the predetermined cutoff.

20. The method of claim 4, wherein the first time point is prior to the administration of the first therapy to the subject.

21. The method of claim 4, wherein the first time point is after the administration of the first therapy to the subject.

22. The method of claim 4, further comprising, continuing to administer the first therapy to the subject when the second score is significantly decreased as compared to the first score.

23. The method of claim 4, further comprising, discontinuing administration of the first therapy to the subject when the second score is not significantly decreased as compared to the first score.

24. The method of claim 4, further comprising administering a second therapy to the subject when the second score is not significantly decreased as compared to the first score.

25. The method of claims 4, wherein the second score is significantly decreased as compared to the first score when the second score is at least 25% less than the first score.

26. The method of claim 5, wherein the first therapy comprises anti-cancer therapy, surgery, chemotherapy, targeted drug therapy, radiation therapy, immunotherapy or any combination thereof.

27. The method of claim 26, wherein when the first therapy comprises surgery, the surgery comprises removing a polyp during a colonoscopy, endoscopic mucosal resection, a partial colectomy, an ostomy, removing at least one cancerous lesion from the liver, or any combination thereof.

28. The method of claim 26, wherein when the first therapy comprises chemotherapy, the chemotherapy comprises FOLFOX, FOLFIRI, a combination of 5-FU and leucovorin, capecitabine, irinotecan, CapeOx or any combination thereof.

29. The method of claim 26, wherein when the first therapy comprises targeted drug therapy, the targeted drug therapy comprises bevacizumab, cetuximab, panitumumab, regorafenib, a combination of trifluridine and tipiracil, an EGFR TKI inhibitor or any combination thereof.

30. The method of claim 26, wherein when the first therapy or the second therapy comprises anti-cancer therapy, the anticancer therapy comprises anti-colon cancer therapy.

31. The method of claim 26, wherein when the first therapy comprises immunotherapy, the immunotherapy comprises pembrolizumab, nivolumab or a combination of pembrolizumab and nivolumab.


SEQUENCE LISTING (31 sequences)

SEQ ID NO: 1 - DNA, 1496 bp, Homo sapiens
  source 1..1496: mol_type = other DNA
gttagagccggctgcgcggcttacggggctcaatcggcggcgagagcggcaggcggggcgggccgaacgcgggtttccggcggggcccggcaggcgccgaggaggaagagcgagcccggacggcgcctctcgaacgagtgtgggcgcgaggcaggatgacgacctcaggcgcgctctttccaagcctggtgccaggctctcggggcgcctccaacaagtacttggtggagtttcgggcgggaaagatgtccctgaaggggaccaccgtgactccggataagcggaaagggctggtgtacattcagcagacggacgactcgcttattcacttctgctggaaggacaggacgtccgggaacgtggaagacgacttgatcatcttccctgacgactgtgagttcaagcgggtgccgcagtgccccagcgggagggtctacgtgctgaagttcaaggcagggtccaagcggcttttcttctggatgcaggaacccaagacagaccaggatgaggagcattgccggaaagtcaacgagtatctgaacaaccccccgatgcctggggcgctgggggccagcggaagcagcggccacgaactctctgcgctaggcggtgagggtggcctgcagagcctgctgggaaacatgagccacagccagctcatgcagctcatcggaccagccggcctcggaggactgggtgggctgggggccctgactggacctggcctggccagcttactggggagcagtgggcctccagggagcagctcctcctccagctcccggagccagtcggcagcggtcaccccgtcatccaccacctcttccacccgtgccaccccagccccttctgctccagcagctgcctcagcaactagcccgagccccgcgcccagttccgggaatggagccagcacagcagccagcccgacccagcccatccagctgagcgacctccagagcatcctggccacgatgaacgtaccagccgggccagcaggcggccagcaagtggacctggccagtgtgctgacgccggagataatggctcccatcctcgccaacgcggatgtccaggagcgcctgcttccctacttgccatctggggagtcgctgccgcagaccgcggatgagatccagaataccctgacctcgccccagttccagcaggccctgggcatgttcagcgcagccttggcctcggggcagctgggccccctcatgtgccagttcggtctgcctgcagaggctgtggaggccgccaacaagggcgatgtggaagcgtttgccaaagccatgcagaacaacgccaagcccgagcagaaagagggcgacacgaaggacaagaaggacgaagaggaggacatgagcctggactgagccacgcgccgtcctccgaggaactgggcgcttgcagtgcgttgcacaccctcacctcccacccactgattattaataaagtcttttcttttacctgccaaaaaaaaaaaaaaaaaa

SEQ ID NO: 2 - DNA, 2020 bp, Homo sapiens
  source 1..2020: mol_type = other DNA
cacctcctgtccgcccctcagcgcatgggtggcggtcacgtgcccagaacgtccggcgttcgccccgccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaatgtttcttcctctgtttggctttgccattgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgcctataatcccagcacgttgggaggccaacgcgggtggatcacctgaggtcaggagttcgagaccagcctgactaacatggtgaaaccctgtctctactaaaaatacaaaattagtcaggcgtggtggtgcacacctgtaatcccagctacttgggagactgaggcaggaggatcgcttgaacccgggaggcagaggttgcggtgagccgagatcatgccattgcactccagcctgggcaacagagcaagactctgtgtcaaaaaaaaaaaaagaatatagatttttaaatggcaaaaaaaaaaaaaaaaaa

SEQ ID NO: 3 - DNA, 2304 bp, Homo sapiens
  source 1..2304: mol_type = other DNA
cggcctgcgtccgccaccggaagcgccctcctaatccccgcagcgccaccgccattgccgccatcgtcgtggggcttctggggcagctagggctgcccgccgcgctgcctgcgccggaccggggcgggtccagtcccgggcgggccgtcgcgggagagaaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 4 - DNA, 2642 bp, Homo sapiens
  source 1..2642: mol_type = other DNA
aatcgctgacatcatccgggggcgggcgcccctgccctgcgggtgactccgacccctggctagagggtaggcggcgtggagcagcgcgcgcaagcgaggccaggggaaggtgggcgcaggactttagccggttgagaaggatcaagcaggcatttggagcacaggtgtctagaaacttttaaggggccggttcaagaaggaaaagttcccttctgctgtgaaactatttggcaagaggctggagggcccaatggctgcaaaatcgcaacccaacattcccaaagccaagagtctagatggcgtcaccaatgacagaaccgcatctcaagggcagtggggccgtgcctgggaggtggactggttttcactggcgagcgtcatcttcctactgctgttcgcccccttcatcgtctactacttcatcatggcttgtgaccagtacagctgcgccctgactggccctgtggtggacatcgtcaccggacatgctcggctctcggacatctgggccaagactccacctataacgaggaaagccgcccagctctataccttgtgggtcaccttccaggtgcttctgtacacgtctctccctgacttctgccataagtttctacccggctacgtaggaggcatccaggagggggccgtgactcctgcaggggttgtgaacaagtatcagatcaatggcctgcaagcctggctcctcacgcacctgctctggtttgcaaacgctcatctcctgtcctggttctcgcccaccatcatcttcgacaactggatcccactgctgtggtgcgccaacatccttggctatgccgtctccaccttcgccatggtcaagggctacttcttccccaccagcgccagagactgcaaattcacaggcaatttcttttacaactacatgatgggcatcgagtttaaccctcggatcgggaagtggtttgacttcaagctgttcttcaatgggcgccccgggatcgtcgcctggaccctcatcaacctgtccttcgcagcgaagcagcgggagctccacagccatgtgaccaatgccatggtcctggtcaacgtcctgcaggccatctacgtgattgacttcttctggaacgaaacctggtacctgaagaccattgacatctgccatgaccacttcgggtggtacctgggctggggcgactgtgtctggctgccttatctttacacgctgcagggtctgtacttggtgtaccaccccgtgcagctgtccaccccgcacgccgtgggcgtcctgctgctgggcctggtgggctactacatcttccgggtggccaaccaccagaaggacctgttccgccgcacggatgggcgctgcctcatctggggcaggaagcccaaggtcatcgagtgctcctacacatccgccgatgggcagaggcaccacagcaagctgctggtgtcgggcttctggggcgtggcccgccacttcaactacgtcggcgacctgatgggcagcctggcctactgcctggcctgtggcggcggccacctgctgccctacttctacatcatctacatggccatcctgctgacccaccgctgcctccgggacgagcaccgctgcgccagcaagtacggccgggactgggagcgctacaccgccgcagtgccttaccgcctgctgcctggaatcttctaagggcacgccctagggagaagccctgtggggctgtcaagagcgtgttctgccaggtccatgggggctggcatcccagctccaactcgaggagcctcagtttcctcatctgtaaactggagagagcccagcacttggcaggtgtccagtacctaatcacgctctgttccttgcttttgccttcaagggaattccgagtgtccagcactgccgtattgccagcacagacggattttctctaatcagtgtccctggggcaggaggatgacccagtcacctttactagtcctttggagacaatttacctgtattaggagcccaggccacgctacactctgcccacactggtgagcaggaggtcttcccacgccctgtcattaggctgcatttactcttgctaaataaaagtgggagtggggcgtgcgcgttatccatgtattgcctttcagctctagatccccctcccctgcctgctctgcagtcgtgggtggggcccgtgcgccgtttctccttggtagcgtgcacggtgttgaactgggacactggggagaaaggggctttcatgtcgtttccttcctgctcctgctgcacagctgccaggagtgctctgcctggagtctgcagacctcagagaggtcccagcaccggctgtggcctttcaggtgtaggcaggtgggctctgcttcccgattccctgtgagcgcccaccctctcgaaagaattttctgcttgccctatgactgtgcagactctggctcgagcaacccggggaacttcaccctcaggggcctcccacaccttctccagcgaggaggtctcagtcccagcctcgggagggcacctccttttctgtgctttcttccctgaggcattcttcctcatccctagggtgttgtgtagaactctttttaaactctatgctccgagtagagttcatctttatattaaacttcccctgttcaaataa

SEQ ID NO: 5 - DNA, 1872 bp, Homo sapiens
  source 1..1872: mol_type = other DNA
catctctaggccccgccccgcgctgcgtgcccacgttgcgccggcctcgcgccagtccgctgggctgcagggactgcggcgcctgagggagtcgctgacgggcacgctgactggaggctggcggacaggcgacagcgacctgcggcagagtcttgctgcgacacccaggctggagtgcaatggcgctatctcggctcactgcaacctccgcttcccggattcaagcgattctcctgcctcagcctcccgagtaggtgggactacaggaccagaggagcgagagcagcaagaaccacacccagcagcaatgtcagcggaagtggaaacctcagagggggtagacgagtcagaaaaaaagaactctggggccctagaaaaggagaaccaaatgagaatggctgacctctcggagctcctgaaggaagggaccaaggaagcacacgaccgggcagaaaacacccagtttgtcaaggacttcttgaaaggcaacattaagaaggagctgtttaagctggccaccacggcactttacttcacatactcagccctcgaggaggaaatggagcgcaacaaggaccatccagcctttgcccctttgtacttccccatggagctgcaccggaaggaggcgctgaccaaggacatggagtatttctttggtgaaaactgggaggagcaggtgcagtgccccaaggctgcccagaagtacgtggagcggatccactacatagggcagaacgagccggagctactggtggcccatgcatacacccgctacatgggggatctctcggggggccaggtgctgaagaaggtggcccagcgagcactgaaactccccagcacaggggaagggacccagttctacctgtttgagaatgtggacaatgcccagcagttcaagcagctctaccgggccaggatgaacgccctggacctgaacatgaagaccaaagagaggatcgtggaggaggccaacaaggcttttgagtataacatgcagatattcaatgaactggaccaggccggctccacactggccagagagaccttggaggatgggttccctgtacacgatgggaaaggagacatgcgtaaatgccctttctacgctgctgaacaagacaaaggtgccctggagggcagcagctgtcccttccgaacagctatggctgtgctgaggaagcccagcctccagttcatcctggccgctggtgtggccctagctgctggactcttggcctggtactacatgtgaagcacccatcatgccacaccggtaccctcctcccgactgaccactggcctacccctttctccagccctgactaaactaccacctcaggtgactttttaaaaaatgctgggtttaagaaaggcaaccaataaaagccagatgctagagcctctgcctgacagcatcctctctatgggccatattccgcactgggcacaggccgtcaccctgggagcagtcggcacagtgcagcaagcctggcccccgacccagctctactccaggcttccacacttctgggccctaggctgcttccggtagtccctgtttttgcagtacatgggtgactatctcccctgttggaggtgagtggcctgtaagtccaagctgtgcgagggggccttgctggatgctgctgtacaacttctgggcctctcttggaccctgggagtgagggtgggtgtgggtggaagcctcagaggccttgggagctcatccctctcacccagaatccctctaaccccttgggtgcggtttgctcagccccagcttatctcctcctccgcgctgtgtaaatgctccagcactcaataaagtgggctttgcaagctaaaaaaaaaaaaaaaaaaaaaaaa

SEQ ID NO: 6 - DNA, 3504 bp, Homo sapiens
  source 1..3504: mol_type = other DNA
atgacgtcgcgttccgtagggctcttcccgggctttggtgggtcacgtgaaccacttttcgcgcgaaacctggttgttgctgtagtggcggagaggatcgtggtactgctatggcggaatcatcggaatccttcaccatggcatccagcccggcccagcgtcggcgaggcaatgatcctctcacctccagccctggccgaagctcccggcgtactgatgccctcacctccagccctggccgtgaccttccaccatttgaggatgagtccgaggggctcctaggcacagaggggcccctggaggaagaagaggatggagaggagctcattggagatggcatggaaagggactaccgcgccatcccagagctggacgcctatgaggccgagggactggctctggatgatgaggacgtagaggagctgacggccagtcagagggaggcagcagagcgggccatgcggcagcgtgaccgggaggctggccggggcctgggccgcatgcgccgtgggctcctgtatgacagcgatgaggaggacgaggagcgccctgcccgcaagcgccgccaggtggagcgggccacggaggacggcgaggaggacgaggagatgatcgagagcatcgagaacctggaggatctcaaaggccactctgtgcgcgagtgggtgagcatggcgggcccccggctggagatccaccaccgcttcaagaacttcctgcgcactcacgtcgacagccacggccacaacgtcttcaaggagcgcatcagcgacatgtgcaaagagaaccgtgagagcctggtggtgaactatgaggacttggcagccagggagcacgtgctggcctacttcctgcctgaggcaccggcggagctgctgcagatctttgatgaggctgccctggaggtggtactggccatgtaccccaagtacgaccgcatcaccaaccacatccatgtccgcatctcccacctgcctctggtggaggagctgcgctcgctgaggcagctgcatctgaaccagctgatccgcaccagtggggtggtgaccagctgcactggcgtcctgccccagctcagcatggtcaagtacaactgcaacaagtgcaatttcgtcctgggtcctttctgccagtcccagaaccaggaggtgaaaccaggctcctgtcctgagtgccagtcggccggcccctttgaggtcaacatggaggagaccatctatcagaactaccagcgtatccgaatccaggagagtccaggcaaagtggcggctggccggctgccccgctccaaggacgccattctcctcgcagatctggtggacagctgcaagccaggagacgagatagagctgactggcatctatcacaacaactatgatggctccctcaacactgccaatggcttccctgtctttgccactgtcatcctagccaaccacgtggccaagaaggacaacaaggttgctgtaggggaactgaccgatgaagatgtgaagatgatcactagcctctccaaggatcagcagatcggagagaagatctttgccagcattgctccttccatctatggtcatgaagacatcaagagaggcctggctctggccctgttcggaggggagcccaaaaacccaggtggcaagcacaaggtacgtggtgatatcaacgtgctcttgtgcggagaccctggcacagcgaagtcgcagtttctcaagtatattgagaaagtgtccagccgagccatcttcaccactggccagggggcgtcggctgtgggcctcacggcgtatgtccagcggcaccctgtcagcagggagtggaccttggaggctggggccctggttctggctgaccgaggagtgtgtctcattgatgaatttgacaagatgaatgaccaggacagaaccagcatccatgaggccatggagcaacagagcatctccatctcgaaggctggcatcgtcacctccctgcaggctcgctgcacggtcattgctgccgccaaccccataggagggcgctacgacccctcgctgactttctctgagaacgtggacctcacagagcccatcatctcacgctttgacatcctgtgtgtggtgagggacaccgtggacccagtccaggacgagatgctggcccgcttcgtggtgggcagccacgtcagacaccaccccagcaacaaggaggaggaggggctggccaatggcagcgctgctgagcccgccatgcccaacacgtatggcgtggagcccctgccccaggaggtcctgaagaagtacatcatctacgccaaggagagggtccacccgaagctcaaccagatggaccaggacaaggtggccaagatgtacagtgacctgaggaaagaatctatggcgacaggcagcatccccattacggtgcggcacatcgagtccatgatccgcatggcggaggcccacgcgcgcatccatctgcgggactatgtgatcgaagacgacgtcaacatggccatccgcgtgatgctggagagcttcatagacacacagaagttcagcgtcatgcgcagcatgcgcaagacttttgcccgctacctttcattccggcgtgacaacaatgagctgttgctcttcatactgaagcagttagtggcagagcaggtgacatatcagcgcaaccgctttggggcccagcaggacactattgaggtccctgagaaggacttggtggataaggctcgtcagatcaacatccacaacctctctgcattttatgacagtgagctcttcaggatgaacaagttcagccacgacctgaaaaggaaaatgatcctgcagcagttctgaggccctatgccatccataaggattccttgggattctggtttggggtggtcagtgccctctgtgctttatggacacaaaaccagagcacttgatgaactcggggtactagggtcagggcttatagcaggatgtctggctgcacctggcatgactgtttgtttctccaagcctgctttgtgcttctcacctttgggtgggatgccttgccagtgtgtcttacttggttgctgaacatcttgccacctccgagtgctttgtctccactcagtaccttggatcagagctgctgagttcaggatgcctgcgtgtggtttaggtgttagccttcttacatggatgtcaggagagctgctgccctcttggcgtgagttgcgtattcaggctgcttttgctgcctttggccagagagctggttgaagatgtttgtaatcgttttcagtctcctgcaggtttctgtgcccctgtggtggaagagggcacgacagtgccagcgcagcgttctgggctcctcagtcgcaggggtgggatgtgagtcatgcggattatccactcgccacagttatcagctgccattgctccctgtctgtttccccactctcttatttgtgcattcggtttggtttctgtagttttaatttttaataaagttgaataaaatataaaaaaaaaaaaaaaaaaa

SEQ ID NO: 7 - DNA, 7390 bp, Homo sapiens
  source 1..7390: mol_type = other DNA
cggaactcgcgggttcggagccgcccgctgaggtcagaaggaggcgtctgcgctgatcgggtccgccgcgcgccagagccagagtcgcagccgaggggagccggggccggagcccgagcccgagccgagccggagcccgagcgagcggcggagaccgtgcccccgcctcggccccgcgccgccgcggccaggcccggcatggaggaggagtgccgggtgctctccatacagagccacgtcatccgcggctacgtgggcaaccgggcggccacgttcccgctgcaggttttgggatttgagattgacgcggtgaactctgtccagttttcaaaccacacaggctatgcccactggaagggccaagtgctgaattcagatgagctccaggagttgtacgaaggcctgaggctgaacaacatgaataaatatgactacgtgctcacaggttatacgagggacaagtcgttcctggccatggtggtggacattgtgcaggagctgaagcagcagaaccccaggctggtgtacgtgtgtgatccagtcttgggtgacaagtgggacggcgaaggctcgatgtacgtcccggaggacctccttcccgtctacaaagaaaaagtggtgccgcttgcagacattatcacgcccaaccagtttgaggccgagttactgagtggccggaagatccacagccaggaggaagccttgcgggtgatggacatgctgcactctatgggccccgacaccgtggtcatcaccagctccgacctgccctccccgcagggcagcaactacctgattgtgctggggagtcagaggaggaggaatcccgctggctccgtggtgatggaacgcatccggatggacattcgcaaagtggacgccgtctttgtgggcactggggacctgtttgctgccatgctcctggcgtggacacacaagcaccccaataacctcaaggtggcctgtgagaagaccgtgtctaccttgcaccacgttctgcagaggaccatccagtgtgcaaaagcccaggccggggaaggagtgaggcccagccccatgcagctggagctgcggatggtgcagagcaaaagggacatcgaggacccagagatcgtcgtccaggccacggtgctgtgagggccccgccgcttgcccgtgacacgcagcgcgttggtgtctccgtgtttgtccctgtgaaaacatgtaacgtctgccttagagccatgaccgaaacttgatatttttttctttcatgagtgtccggcatctgctggtcttcattgtgaaacgtgccagtcgtgctttgtgaaaaataacaaagtggtcacagaaatttgtgatctgaaaacccggctcccttccccacaaggctcctgggcctccgggaagacgggcccctgtttgccatctcgggggtgttccctgtgggagggtgagtgggtgaggccgagcctgctgcgtgtggagcctcgagtgggccctggctgccactaccgtacagaggccgtgtcgcgctgggctgggcctgggtggcctctgtctttgcatctctgagaaggagtcgggtggtaacggttggggtcaggaagaattctgccaagtatctttactgtcattctgaccatagcctctttgttcccgcattcgaacttttggttcttactttgctgctcgtttagtccctggggatttcagatcttaggctgttgtttcaccgtatgggagggttgatgtgagcttgcttggagacacacggtgcagcatcagggaccttcccaggccccagcaaattcaagtcggtctgcagacctctcagctacccgcgggacctcttgtaacccatcggcatcttccaggaatccgccgagtgacttgaggaagatgctaacgcagtaaggtctgtgctgggccaagagcagctttgaagctccagagaaccaccccgtcaggttccttgtggaagctcccctcatccgtggtgcagcaggctgagcactgcgcgtttgccacgtgctgcccgtgacagcacattgagccacagcatttgtagacaggacagaggggtgcctgccccctgcccctgctggcacatttaacccttgtcccctgacctcagttctgtgccccaccaaatgcccaggggcaagaggccaccctggaagctgccaatcttccaaggtgggtgtggggcacggtgggggcgggcagctcccaggcccttgggcaggctggggtgacggcagaggccacagcaccagctctgacaagtcctatcatcctctgctcagcagtgacctccctggccccactttgcccagagtttggggtccccccaggtatagctataggcggcagtgcctgtccctggcctgccttgatttcagccacacccctgcagccctgcatcccagctctggggtgtgcagaggtttgtgtctccagggaacccacggctggagagaaatagggagatgcaggaagtgggggcccatggggcccccaagaagcggactctccaaggggtacccccaccccgctaccttccccacggacgggcccctcctggagcccataccctcctgtgaggccattccagtgtcttctagaaagactcgcttgccaggagtgcgttctttgttgaaaaatgccctgaagcgaaaagatgcaggtttatatggaacccccaccccctcccccactctcccactctgttcgttctgaatgtcttcacgagcgtgcatcagggcgcctggctcccccacctcagccagtgagtcagacacgggtttcgcagccatgtttcctggctccgaggacacgggtggcaggcccgttgcagcccagagccactggtccctacagggcgccgccacaccagcaggaaggaggatggctgtgtccggagcctggcggggaggcggcctccccagtatgtgagtgcagggatctgccagaaccacctggccctctgtagggcgtttaactggaaataccctcactgccaagtggagactggggcgtgtgccacattgccagccaccaggaaagcttttctttttcttttttttttttttttaaacaccaagagcacgtatagcatgggggaaagaacctaaatgtctctctgtcctgtgagctggtgaaaaacccagcatgagaacgcagtgtcaggtgtgggactccttctgcccctgcagtgggtgttacgggcggtgtgccctggcgagcaagctttgattcttggttctttgagctcgtttcagaggctgagtccccacatcagctttagttcttggacttccctgtattaagcaagaattaggagaatggctgtccctgcaggcgcctcccgtaaatcctgagctctctggcgcaatctgaaacttctcttctgttttctttggctgtatcagccgaaccaggagaggcctgggctgcgactaaggagaaagaaatcgggggtttctgagagcagatggtgcctttgtgggtgcagggcttttgtggaaattgtcagcctctacgggcagagtccggcatcccctccccagactgcctgctgtcaaaccacggagcagctggagcctgccctgtccacggcccgtttccacccgggcatgttcgtctctcatgacttcggcagaggcccctggtggccttcagtttcagtttctcatccaggaaggtaaccttgggcattggcagtgggtttccctatggcttggatccagattagaattgatctttgttttcactttccatagttaataacatgcaaaataatgagaagaatttattttaaggtgacagctatactggtccaacatcgcctgcttattgtcagggtacagaagtttaatactttcttaatccagtttttcaaacttctccctgtagaccgtaaggatgaattccacaataggatcctttttaaaatcgattttaaattgttgcctagtcctgccaaggttattatgtgcatctgttatttttccaatacatgtaaacagttgcagcatgatgctttgtttaatgtcctgttcttaagctcgttagagccagttttgaaacgtttggtcttaccgtgaacggaggctggcttggcttagccacgctgatgagtaagtgagggatgtctccatcttgagatcaccaggcaagagagttgcctgcaccaggtaagaggccaaagcccctggggtaacagtccccaccgctacccgaggtaaaacaataaaagctatgtggttgagctcaggcctctcgtgcctggtgtcagagaaggcagagcccacagtaggtgcacggtgcaaggccctgggagggcactggccagggaaggtggtatagatggccctcagattgcggggccccgagcagctccccactctgcccgtccaccttccctggctccagcctcattctctctttagtttaactatgcaaagagaggaggttgagagtgttctggcagctggagctcttttccttgtccttcctgccctccgatggggccacctgtgtcggggcagcagtgtccatgtttatggagatcagaggtgtccccactgtgtggctggactgtactctgctgcccgggtagccaggagtctctccctctctcccctgccgcctgcctggtctcatgggcctccttcacacacccctccctgtggatcgcctgcctgggcccagagcaggggaactggagtttgtgagtgagcagagcaggttatgtgcagacagggaaacgagaactttggacctggctttctgagtccaggtgagagctgtgtggccccccgatgccactctgcccgccggagggatgtgcctgctgagccttttccttccacgccgcctctcactgccaggccagcggcttccgctgagactcgctggagaggcggctcccgtgtccgtccaccgagcactcagatggatgctgatcaccagggccgagggggctcccagaaggaccccaggccctggggagggtggctgtgggaggccaagtccactgcccggaagtcttgtcagccctaagccagggaagcctggagcgtggcctggcgggtctgggtggacaccgtccccactccggactcccagcacaggggaggatacctgagcctgtatggccctgtagccctgggcagagctgggcctgtcgtgtgttcctgcctggcaggtgcaggtgctggccatctgcaggtggaaggaggtgggaatcttggattttttgtttttttttgtttttttttttttgagatgaagtctcgctctgacacccaggctggcgtgcagtggtgtgatctcggctcactgcaaactccgcttcctgggttcaagtggttctcctgccccagcctcccaagtagctgggattacaggcatgcgccaccacgctcagctgatttttgtatttttagtagagatggggtttcaccatgttggccaagctggtctcaaactcctgacctcaagtgatctgcccgcctcggcctcccagagtgctgggattacaggcatgagccagtgcacccggcggaatcttggaatttttatagacagcacctcagtttctgactccagccgcacacctcctgcctctgccagcaggggttgccgccagaccagagccagggccaggtccctgcgtccatcccccccggtaggatggacgtgagccatccttctaggggacttttttcagtgtgcgactcgtctctgttaggtggtaggagccagtttgtgtggcctgtgccacgctccacagtgcgtggctgggctctgtgtgtggcctgtgtcccctgtccctgcaggacccagcaggcatcgtggcgtgacagctgtgtccaagccactgcccgggcatcccatcacccaccagggtgcacggtctctcctgctgggggctttctgtcgcatgtgtgtctcctgtcgactctgcagtttgttctcagagcagaatgtttcctgttctcagtgcacaaagacactggttttcaatcggcgtctaaaaccacgttcctgcctttcattgcaacacggtgtgttcatttgtttaaaacagtttaatgagtaagtttagatgactggtcaatatcttaaaaatgtatattagtaagaagttcttcctggaatttttctttcgattctggcagaataaacaggtgtttttagttttcccactgtctgagccaagcaggaccctgtcccagagcaagagatgtccccttccatctctgacccttgcctgggacaagctttgatggggggccccagcttcaaggctgtggtgggaacagcacccccaaatgccagcctctcctttcttcccatccaccagtatactgcggggccatttctggtctttgtccaacaggaaacccatttctggtgggatatgccttccagtgccacagggccactcaccccatgcatctctgtcctgcccgtcagtgctgggacggacagcaagggcaagcccagtgtctggcggataggtgggtgggaacagagaggggagaatgccgtcctaagcttctgcttggggatcccccacacgacctgggtactgcctgggaaacctgtcctaagtaaaactatggacctcgcctcgcccaccggcctgcgaagccagcatctccgtgaaggtggatggaagcgcctttgtcctcattttgagctgcaagctgggtcagcggctctgaagccctcgagtgactttctaacccaagacccagcccctggcaggaggagggtgggtgcagggctggtgggacaaaaagaggcctcagcaggcctggaagacccttccagtacatcccacagcgtgtcgagcagctgggagaacctgtgtcaagctcgagccgtcataggtccccatgaggtgtctgaagccccttcttggtgatgggaggcagaggtgctgacgttctggagcatggacgtgagtcctcagctggctccgcgtgggcccttggagggtgccaggtgtgtggtgaccttctggatgcctttaacttcatggctgcgtcattcctgatttagaactttaaccggagcttcatctagtgattgcaaaactggaccaatgggaggacggcggcgcagcccgctccctccgtggaatggagctcagctcttcggaggcatcaaagcacctgtcgcctccgtggtccccctgctgagggagtgcggcctctgcaaggttcgggggtggcttcgtttgcctggagtggccggccctgcttgtgccatgtggatgtttgtgagcctcggtcctacagcactgtgtaggctgcatctgtttcgtgctggtcctgttgacttgtatgatatccacaaataaatattttcatggcggtcgtgttgaaaaaaaaaa

SEQ ID NO: 8 - DNA, 949 bp, Homo sapiens
  source 1..949: mol_type = other DNA
ggaaggggcggggcgaacggaagccgggaaggcgattcatagctcgcggggtacgggcgcgcgtgcgcactccgcagcccgttcaggaccccggcgcgggcagggcgcccacgagctggctggctgcttgcacccacatccttctttctctgggacctggggtcgcggttacttgggctggccggcgaacccttgagtggcctggcggggagcgggcctcgcgcgcctggagggccctgtggaacgaagagaggcacacagcatggcagaaaaccgagagccccgcggtgctgtggaggctgaactggatccagtggaatacacccttaggaaaaggcttcccagccgcctgccccggagacccaatgacatttatgtcaacatgaagacggactttaaggcccagctggcccgctgccagaagctgctggacggaggggcccggggtcagaacgcgtgctctgagatctacattcacggcttgggcctggccatcaaccgcgccatcaacatcgcgctgcagctgcaggcgggcagcttcgggtccttgcaggtggctgccaatacctccaccgtggagcttgttgatgagctggagccagagaccgacacacgggagccactgactcggatccgcaacaactcagccatccacatccgagtcttcagggtcacacccaagtaattgaaaagacactcctccacttatcccctccgtgatatggctcttcgcatgctgagtactggacctcggaccagagccatgtaagaaaaggcctgttccctggaagcccaaaggactctgcattgagggtgggggtaattgtctcttggtgggcccagttagtgggccttcctgagtgtgtgtatgcggtctgtaactattgccatataaataaaaaatcctgttgcactagtgtcctgccatcccaaaaaaaaaaaaaaaaaa

SEQ ID NO: 9 - DNA, 510 bp, Homo sapiens
  source 1..510: mol_type = other DNA
tgaggctgccttataaagcaccaagaggctgccagtgggacattttctcggccctgccagcccccaggaggaaggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatga

SEQ ID NO: 10 - DNA, 1646 bp, Homo sapiens
  source 1..1646: mol_type = other DNA
ggcggggccaggagagaaagctttgtggtttggtctcagggaagtagcaggcgccggttgagagaactacggccctgtcggaaggtaacctccggtgcaaacgaccatcggcggcaggcgagcggtacgcttggcgtccgggccttcctgggcccgtctgaggaaacttgctgctcgaggccaggctgcctaggacctgtcccttttttctatactggctcccacatccgggttttttctccgggacggcccttcggatgcttgggccaatgggaatcgccatttagggtgctccgcccaccgggtcgcgtagagcatcctggaagtcgtagtaaatctctcgagagttctctccgcacgcgggctggagaagcgggtcctacgcacgctttgttgtcgcgctttgcctccgtccttgcccctactcccgccttacctgacttccttttcggaggaagatccttgagcagccgacgttgggacaaaggatttggagaaacccagggctaaagtcacgtttttcctcctttaagacttacctcaacacttcactccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaaggcaccttcgtggagcgggaccgcaagcgggagaagaggaagcccaagagccaggagaccccggccaccaagaaggctgtgcaaggcgggggagccacccccgtggtgggggctgtccaggggcctgtcccgggcatgccgccgatgactcaggcgccccgcattatgcaccacatgccgggccagccgccctacatgccgccccctggtatgatccccccgccaggccttgcacctggccagatcccaccaggggccatgcccccgcagcagcttatgccaggacagatgccccctgcccagcctctttctgagaatccaccgaatcacatcttgttcctcaccaacctgccagaggagaccaacgagctcatgctgtccatgcttttcaatcagttccctggcttcaaggaggtccgtctggtacccgggcggcatgacatcgccttcgtggagtttgacaatgaggtacaggcaggggcagctcgcgatgccctgcagggctttaagatcacgcagaacaacgccatgaagatctcctttgccaagaagtagcaccttttccccccatgcctgccccttcccctgttctggggccacccctttcccccttggctcagccccctgaaggtaagtccccccttgggggccttcttggagccgtgtgtgagtgagtggtcgccacacagcattgtacccagagtctgtccccagacattgcacctggcgctgttaggccggaattaaagtggctttttgaggtttggtttttcacaatcaaaaaaaaaaaaaaaaaa

SEQ ID NO: 11 - DNA, 2813 bp, Homo sapiens
  source 1..2813: mol_type = other DNA
ctccacgctagcgccgcccaggctggcacaaaggaggaagcctagtcccgcccctgcgtgcggcgcttctcccaggccccaccttccatccagtgccctggaccctcggctgggtagcgccaccagagcgaccaaacgtcccgcgccttccaggccgcactccagagccaaaagagctccatggcggcggcggccaagcccaacaacctttccctggtggtgcacggaccgggggacttgcgcctggagaactatcctatccctgaaccaggcccaaatgaggtcttgctgaggatgcattctgttggaatctgtggctcagatgtccactactgggagtatggtcgaattgggaattttattgtgaaaaagcccatggtgctgggacatgaagcttcgggaacagtcgaaaaagtgggatcatcggtaaagcacctaaaaccaggtgatcgtgttgccatcgagcctggtgctccccgagaaaatgatgaattctgcaagatgggccgatacaatctgtcaccttccatcttcttctgtgccacgccccccgatgacgggaacctctgccggttctataagcacaatgcagccttttgttacaagcttcctgacaatgtcacctttgaggaaggcgccctgatcgagccactttctgtggggatccatgcctgcaggagaggcggagttaccctgggacacaaggtccttgtgtgtggagctgggccaatcgggatggtcactttgctcgtggccaaagcaatgggagcagctcaagtagtggtgactgatctgtctgctacccgattgtccaaagccaaggagattggggctgatttagtcctccagatctccaaggagagccctcaggaaatcgccaggaaagtagaaggtcagctggggtgcaagccggaagtcaccatcgagtgcacgggggcagaggcctccatccaggcgggcatctacgccactcgctctggtgggaacctcgtgcttgtggggctgggctctgagatgaccaccgtacccctactgcatgcagccatccgggaggtggatatcaagggcgtgtttcgatactgcaacacgtggccagtggcgatttcgatgcttgcgtccaagtctgtgaatgtaaaacccctcgtcacccataggtttcctctggagaaagctctggaggcctttgaaacatttaaaaagggattggggttgaaaatcatgctcaagtgtgaccccagtgaccagaatccctgatgttaatgggctctgccctcatccccacagtcttgggatctcagggcacaatggctggacatgggtgggctctgatgcagaactttctcttttgaatgttaagaataactaatacaattcattgtgaacagaagtccttaagcagaggaattggtgtgccttaaagatacaatctgggatagtttgggggaacttgtagccagaatgccctgttcatgctgagcaaagttcagcaagtagagcagagtttggcaggcaggtgccaggaactccccttcttcctggagtgccttcattgaggaaggaaatctggcccttgggtttcctggttccactgctactgacccagaggggaatgagggctgagttatgaaaagataacttcatgaagacttaactggcccagaagctgattttcatgaaaatctgccactcagggtctgggatgaaggcttgtcagcacttccagtttagaacgcaatgtttctagagacatattggctgtttgttttgatgataaaaggagaataagaaaaggcatcactttcctggatccaggataatttttaaaccaatcaaatgaaaaaaacaaacaaacaaaaaaggaaatgtcatgtgaggttaaaccagtttgcattcccctaatgtggaaaaagtaagaggactactcagcactgtttgaagattgcctcttctacagcttctgagaattgtgttatttcacttgccaagtgaaggaccccctccccaacatgccccagcccacccctaagcatggtcccttgtcaccaggcaaccaggaaactgctacttgtggacctcaccagagaccaggagggtttggttagctcacaggacttcccccaccccagaagattagcatcccatactagactcatactcaactcaactaggctcatactcaattgatggttattagacaattccatttctttctggttattataaacagaaaatctttcctcttctcattaccagtaaaggctcttggtatctttctgttggaatgatttctatgaacttgtcttattttaatggtgggttttttttctggtaagatttagacctaaatcgcatcatgccaacttgtgactttgagactattcatcaagaatgaggatatagtagccatgacatagcttgagctatagcctttaattccttactttggctatgggtggagggtgagtttgaagaggttctgattttcttgtaacctgggaaagccatgaccttgtgcccgattctttcagattgctttgggtaataaatattggtggtggtatctgactcatgctgctgtttatggtcctgtttagtggggaatggactcaggttacccatttcccagagggaaggatcccaggatttttgaaggttacatattttctgtaccaaatataatttcattgacatgaattatctctaatcctcatgacaagccacatacacaatcattttgtagataaagaagatataaatgccagaggagaccttaagattgtcttacaacacaacccttcagttaacgagagagg

SEQ ID NO: 12 - DNA, 1296 bp, Homo sapiens
  source 1..1296: mol_type = other DNA
tccgggggagcggaactgcaagaggaaaggctcgggtaggcttctgggagcgaccgctccgctcgtctcgttggttccggaggtcgctgcggcggtgggaaatgctggcgcgcgcggcgcggggcactggggcccttttgctgaggggctctctactggcttctggccgcgctccgcgccgcgcctcctctggattgccccgaaacaccgtggtactgttcgtgccgcagcaggaggcctgggtggtggagcgaatgggccgattccaccggatcctggagcctggtttgaacatcctcatccctgtgttagaccggatccgatatgtgcagagtctcaaggaaattgtcatcaacgtgcctgagcagtcggctgtgactctcgacaatgtaactctgcaaatcgatggagtcctttacctgcgcatcatggacccttacaaggcaagctacggtgtggaggaccctgagtatgccgtcacccagctagctcaaacaaccatgagatcagagctcggcaaactctctctggacaaagtcttccgggtggaggcagagcggcggaaacgggccacagttctagagtctgaggggacccgagagtcggccatcaatgtggcagaagggaagaaacaggcccagatcctggcctccgaagcagaaaaggctgaacagataaatcaggcagcaggagaggccagtgcagttctggcgaaggccaaggctaaagctgaagctattcgaatcctggctgcagctctgacacaacataatggagatgcagcagcttcactgactgtggccgagcagtatgtcagcgcgttctccaaactggccaaggactccaacactatcctactgccctccaaccctggcgatgtcaccagcatggtggctcaggccatgggtgtatatggagccctcaccaaagccccagtgccagggactccagactcactctccagtgggagcagcagagatgtccagggtacagatgcaagtcttgatgaggaacttgatcgagtcaagatgagttagtggagctgggcttggccagggagtctgggaacaaggaagcagattttcctgattctggctctagcttccctgccaagattttggtttttatttttttatttgaactttagtcgtgtaataaactcaccagtggcaaaccagaaactgtcctctttgattggggaatgaagttgggaaagtcactagcattttccttggatccagtcctgtcagcatgatgcctccatgaataagagtgaacttcttgtaaagtgaaact

SEQ ID NO: 13 - DNA, 6738 bp, Homo sapiens
  source 1..6738: mol_type = other DNA
ctgcagacgaggcaggcggaagaggcgggacttcgcgggtgacgtcatcggggcgccggaggcccggggcgcctgggaatttgaagcaaacaggcagcgcgcgacaatggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttgagtgcttcagatacatttttcagatacaatgtgaagacattgaagatatgtggtcctcctgaaagtcactggctggaaataatccaattattcctgcttggattcttccacagggcctgtgtaagaatgggttctggagttctcatggtctttaggaaatattgagtaatttgtaatcaccgcattgatactataataagttcattcttaagcttgctttttttgagactggtgtttgttagacagccacagtcctgtctgggttagggtcttccacatttgaggatccttcctatctctccatgggactagactgctttgttattctatttattttttaatttttttcgagacaggatctcactctgttgcccaggatggagtgcagtggtgagatcacggctcattgcagcctcgacctcccaggtgatcctcccacctcagcttccagattagctggtgctataggcatgcaccaccacgtccatctaaatttctttattatttgtagagatgaggtcttgccatgttacccaggctggtctcaactcctgggctcaagcgatcctcctgcctcagtctctcaaagtgctgggattacaggtgtgagccactgtgcccagcctaattgcagtaagacaaaaattctagggcaccaagaggctaaagtcagcacagcttttcttgtgtcctgtattctctgtctaatgtgttgcccaaataatacctaattgttagccattcccctccatctctggcctaaaagtgatagtccaggtatccacatgggctggttcccagaactgccattgctcactctccaaagaggggaaggtggggaaggggaaggtgactatagctcagctcctgagctagtatctggctgttatttcaacaaccggagttggggtttgggctcattttttcccctagccagcaattatggaccagtagtaacacaagtgacagcttcctgtgactgacttcacaattaggaggtctaagattccatttgggtatttgcttaaggatcccacataattgtcccaacggtcattagtagaggggaggtaagccttcattaataataaagagaaagcccacattcaaggtggtgtttgagcaggggcagggtgagggctgtcccggtgctcattgcaccagcacactcacattccttctcatttggggcccacctgcaggaagtggcacaggatcagccatttccccacccttgtcagctgatggcccactgttctttaatgactcagaggaatgcctaggatttttttttttttttgagacagaatctcactgtcgcccaggctggagttcagtggcacgatctcggctcactgcaacttctgcctctctggttcaagcagttctcctgcctcagcctcccgagtagctgggactacaagcctaggatttttaactcaggtttttattatattccctcctgaagtttttacttcaagagcttctgctctaaagtccaatttgggcttcatgtccccagtgctgcatctccagggaaatgctgtctgtgggagagaccaactctcaaggaagaagtggccacagaaggagcaggaagggagttggccctcagggctactctggggaagccaaaagtcatgaaggggagaagaattttctgacaaaaacttgcaggaatctcttaggtgtcttcagtgttggagtgatatgttgagaggcctttggagtgatgtgctgaggtctcaggcgcccacctccctggctgtcacttccatgtgtcagtggttctcccactttagcaggtatcagagtcacctggagtcttgtcaaaacaggtaccagccccacccgcagcgtttctgactctgggtagctctgggatggggcttgagaatttgcgtttccaaaaaggtcccaggtgatgctgcggttgcctgcgcagggactggactttgagaaccacttcactggttattcacatttctgcctctgcagtgagacagccttgaggtctgcctcctgctaagagtcacatgctcctgtcctttagaaatgtgggctcctgccatctccaggacgcaggcactgttcctgttgatgaaccctatttcacaggacccctgctaaggtgatttgaggggaaatgagaggaggctcaaataatcacccagcccctgccacttactgaaagtgtaggtccttgtgccccacaccatcagagtttctgcgttagcagatttgtggtttgcccagcagcctgggcgtgtgcatttctaatgggtgcctcaagtgatctgtttctgatttgtatttctattgtgaagagtcagcccagtactgcaggcctcttacctaagcagaatcccagtctggcatcaaagctttagaggacaagttgattcaggcagagaagaacttgggctatacaagcgctgttcttcagcattgaagtattttggaggcattagatagtttaaccctttctcagtcaaggaatatttacagaacatgatctctgggcattgtaactcctggtcttagtggggaatatagggaccccatgtctccatggggtgcacagaatgtctgtgagactgatggagtggagaacgccatcccccagcctctccagctactcgaggcattctgtagaacataagcccatagattgtgtgtgtgtgtgtgtgtgtgtgtgtgtgtgtgtgtgtgtgcatgcgcgcgcgtgcgcactggaggaacctaagaaactatttggtgcacttcctcttattttagagctcccaaagtgtagctccagaatcgtaaagggatatgctcagtctcacagccagcctgtggatctcagtcccaacactcacccttgtgctactgagtcagctctaagaaaatctgccaaaagtaggccgagggctggttttttgttttgttttgtttgtttgatacagggtcttcactctgttgcccaggctggagtatatcatggctcactgcaaccttgacttgggctcaagcgatccgctcaagtagctggaactactctcaagtagctctcaagagcctctcgagtggctggaactacaggcgtgcaccaccacagctggttaatttttaaaattttttgtagagacggtggaggaggttctcactgtgactcagtgtgtgcccgacagcagagcccacaccactccagttgcagtggttgccatctgggtcatcagacctggctgtcaggggtgcagccacaggagagccaacagcagagggtgctggccgctgagctagctgctaatgctggcctgggtgcagttctcatccaaagtacccggtgggtgggagtcactcagtaccagttccgagcctgaacccaaactctcgtgtttctgctcacccctctctggcttctgccaccacatgggaagaatatgccctggttagcccatggcttctgaagagcaagagaaagtagagcagagcctactccagcctcccccgtccaatgtatgaaagccccagctgatctgtaagcctgggagcgtgataaatgcttagtagtgcatgccatggagttccagggtggtttattacacggcaatatctagctaaatacatttaacttgctgcagctctctggatccagcctggttaccaggaagacaaaaactgggctccaccaggaaccagtcttctgccttcccaaccatcacctctggctgcatcagcgatctctcccagcgaaatagctgcttggtcttgtgtgaatcctgtactttaacacagtggaccaagtgtcagtcattgaaaatgaccatgagtaaccctgtggactctctgcagcttggttcctttgccccttaacaggtgggtatgaatcgtgtcttcagtgccagggctgaatgagaaagggcattcctttttgaaggaatctgatactaaacacaaagcatgagaaaaatcaggacttgttggagttatatttttaaaatatatattttaacagttatatatattagatataatatataatagtatatataaataatactatattgcccaggctggtctcgaactccttagctcaagtgatcctcctgccttggcttcccaaagtgctaggattacaggtgtgagccactgctcccggcctgttggagttctttacatttattttataatcaatgctgttttattaaatgcggattttattttggattacaggatgtagaatgccatatttttcttagatcatagggcctttcacatttgtaatttggccttgtatgagttaccctgcaatccctttgttttccccataacccttccaaaggaaggccgcaatagaaatacaaagagaaacaaaataattagaatattttttaacttctaaagttcaaggttttggcataagtctggtttagaagcacatttgcctagccctttccttcccaccaagggggaaagtcttcctctagacaagaggcagagggctcctcagagtcagatcctggtgtgggctctcacgtgctgctgctgaatcccagggaaggagggaggaagggcagttgacacccaaaataagggtggggaactgtcagcagaggaggtctgtgtcatgtttttcagcgctggggttggggggagcccaggagagcaggaagatccagagatccctcgccccagctcggccatgtgtgtctgggacagagcctgaggtggcctgagcttcctgtggctccagagtaacattatagagaagctgaattctcctgtttttctgaaaagggcatgggagttagctgagaagcagacctggtgggcctgagagtctcaatcgtcaggtaaggacagtcagtgggaagtggacgggccgcacaaccaaggttctcatgaggacaaccatgtcttcgggggtgcccttgtgcacagacagctccatagtcctgcctccaatgtcccaacactgcattgtctccctgcacttagcagccctgcagggtgagacttggggaggatcctgaaatgattgtatttaacaagacatgctgtccttgtttacctggaacctagcaatgttgttttctgccacaacttgaatagatacttgaagcagagatgatgttgagttaaaaaaaatatatacataaaaatatgggttcttttcaacctgaatagatggcctaaaaattcaaa

SEQ ID NO: 14 - DNA, 1779 bp, Homo sapiens
  source 1..1779: mol_type = other DNA
cggcgtgccctggggcggcgcgggcgcaggggcgcgtgcgcggcgggctgtcgttggctggagcagcggctgcgcgggtcgcggtgctgtgaggtctgcgggcgctggcaaatccggcccaggatgtagagctggcagtgcctgacggcgcgtctgacgcggagttgggtggggtagagagtagggggcggtagtcgggggtggtgggagaaggaggaggcggcgaatcacttataaatggcgccgaagcaggacccgaagcctaaattccaggaggttgggatgaatgggttccggagagcagagtactcaaatacgtggacaccaatttgcagaaacagcgagaacttcaaaaagccaatcaggagcagtatgcagaggggaagatgagaggggctgccccaggaaagaagacatctggtctgcaacagaaaaatgttgaagtgaaaacgaaaaagaacaaacagaaaacacctggaaatggagatggtggcagtaccagtgagacccctcagcctcctcggaagaaaagggcccgggtagatcctactgttgaaaatgaggaaacattcatgaacagagttgaagttaaagtaaagattcctgaagagctaaaaccgtggcttgttgatgactgggacttaattaccaggcaaaaacagctcttttatcttcctgccaagaagaatgtggattccattcttgaggattatgcaaattacaagaaatctcgtggaaacacagataataaggagtatgcggttaatgaagttgtggcagggataaaagaatacttcaacgtaatgttgggtacccagctactctataaatttgagagaccacagtatgctgaaattcttgcagatcatcccgatgcacccatgtcccaggtgtatggagcgccacatctcctgagattatttgtacgaattggagcaatgttggcttatacacctctggatgagaagagccttgctttattactcaattatcttcacgatttcctaaagtacctggcaaagaattctgcaactttgttcagtgccagcgattatgaagtggctcctcctgagtaccatcggaaagctgtgtgagaggcactctcactcacttatgtttggatctccgtaaacacatttttgttcttagtctatctcttgtacaaacgatgtgctttgaagatgttagtgtataacaattgatgtttgttttctgtttgattttaaacagagaaaaaataaaagggggtaatagctccttttttcttctttctttttttttttcatttcaaaattgctgccagtgttttcaatgatggacaacagagggatatgctgtagagtgttttattgcctagttgacaaagctgcttttgaatgctggtggttctattcctttgacactacgcacttttataatacatgttaatgctatatgacaaaatgctctgattcctagtgccaaaggttcaattcagtgtatataactgaacacactcatccatttgtgcttttgtttttttttatggtgcttaaagtaaagagcccatcctttgcaagtcatccatgttgttacttaggcattttatcttggctcaaattgttgaagaatggtggcttgtttcatggtttttgtatttgtgtctaatgcacgttttaacatgatagacgcaatgcattgtgtagctagttttctggaaaagtcaatcttttaggaattgtttttcagatcttcaataaattttttctttaaatttcaaagaacaaaaaaaaaaaaaaa

SEQ ID NO: 15 - DNA, 7827 bp, Homo sapiens
  source 1..7827: mol_type = other DNA
gtagtcttgacgtgagctagctggcatggcggcctgcattgcagcggggcactgggctgcaatgggcctaggccggagtttccaagccgccaggactctgctccccccgccggcctctatcgcctgcagggtccacgcggggcctgtccggcagcagagcactgggccttccgagcccggtgcgttccaaccgccgccgaaaccggtcatcgtggacaagcaccgccccgtggaaccggaacgcaggttcttgagtcctgaattcattcctcgaaggggaagaacagatcctctgaaatttcaaatagaaagaaaagatatgttagaaaggagaaaagtactccacattccagagttctatgttggaagtattcttcgtgttactacagctgacccatatgccagtggaaaaatcagccagtttctggggatttgcattcagagatcaggaagaggacttggagctactttcatccttaggaatgttatcgaaggacaaggtgtcgagatttgctttgaactttataatcctcgggtccaggagattcaggtggtcaaattagagaaacggctggatgatagcttgctatacttacgagatgcccttcctgaatatagcacttttgatgtgaatatgaagccagtagtacaagagcctaaccaaaaagttcctgttaatgagctgaaagtaaaaatgaagcctaagccctggtctaaacgctgggaacgtccaaattttaatattaaaggaatcagatttgatctttgtttaactgaacagcaaatgaaagaagctcagaagtggaatcagccatggcttgaatttgatatgatgagggaatatgatacttcaaaaattgaagctgcaatatggaaggaaattgaagcgtcgaaaaggtcttgattctgagaatgaatttggttagttgcagaagatacattggctctaagaggatatattttgagaccaatttaatttcatttataagaacatagtaattaagtgaactaagcattcattgttttattaatactttttttctaaaataaaacttgtacaccagtttattactctaaaaagagaattacacatgccaaatggaccaatgtccatttgcttattggaggcaaagctacaatagaagtcagagcatcaccagaatggtctttaatgagcatggaacctgagcaaagggaataggtgggatgaattttttttttaattgtgaaacaattcataagcacaatatgatttacagaataataaacattcatgtacccactatcaggttaagaaatagaacatttattaatatgtaggaatgttaagaaataaaacatttaataagatctcagaagactccagtaaatctgcaattgtatctctctcctttttaaatgtaaatatcatcttgacttgttaattattcccttgcatttcttttagtttactgccaacacatatattcttcaacaatatatttaattttgaaaaacctgaaaaaaaaaacctgttagcaagtataaaggggcagtattactattattgcatgaaggcttcaagggaaacgttacagtctttgggtcatagtctggcttcagcttcctctgagagtttacagaggccaattttgagcaaattcatggctaaggttatgagtgagttctgctaaacagaaggctcaccacaaggtatctggcaggattatactgggtagctggatgttgcagaaatgtggttagaggaagtaaactgttttttgatgctcacagcatgatgaatcaaactctgtatcttaggattaggttaaaacaatacctttggtatgatatgagtgttgttgctgatccatgcagcatggattggaaagctggggtataagcacacatgctaaagaaaaacatgtaatttggtccatactcacctggatatactgttcctcaggttaaaaaatacagtactatcctaaatcttgaaggcaactctcagcctatccattgagttaccttcagatctgccctctggttcctagctgtcttgggactaacttctttcctgcgctcagctgttttctggattccatgttttccattttattgagtactaacttgttttgctgcagcacatcctttggtagcttctagaggaagtttgtgtggaggtaaaatttttgagaccttgcatgtctcatgtttgattgatactttatacgtttaggtaggaggtaattttccttcaggactttaaaaatattgttgctccattttctttgtttctattgttgtattgagaaatccaatgccattttgatttccccatcataaatttcatgatgatgtgtcttggtgtgggtctatatttatccattgtattgggttttaggtgaacccttccagatagtaactcatttctgtcagttctgggaaacacttagcattggttgatgatttattctctgctgctttgttctcccaactattatttggatgttggatatccagcactgggtatctattttcttacctccctcccttgaccccagtctctgttttttagctctttagctcaatcttccaactctttgctattgtattttaaaatcttaagaccccttcttgatttgtagaagttccttttcttacaaccaaaaagcctttatctatggatttgttcacagataaggggtattcaatatagtgtatttttttttcatttaaaattgtttgcgcatctatttcctccaaatttctttctgtatttattttttgttgtctatatttcagacttttccaggatatctgataatctttggctgtcttcttatggttgaaagagggactaaaaagcttggaaagcctttgggttgtgggaaggggctgtctttaggattatctgaatgggcttttttgggagtcccctcctccacatgaatattttggttttgtcagattccctagaatagaggcttccaatctccttcctggaggggtctgtccaggaaggagattgtctaggggtctgtcagacagcagctttcagctacttccttgatctttttcactaatgattatatagtcatctaactactgtcaacaagtaatagatatcctatccttcacttgtttagattatttgctgagataacctctcaaaagaacctctcaaaataaaaggttaacaagagcctatatcttatatttttcttctctttatcttgttagaagatagctattaaaacctgttctttttctgtcttgataaacacacttcaatcttggtagaatggtagatgggacagtatattttaggacctaaagctctgcaaatgtatgatcagcttgtaagtacaggtgctcaaaaacatgtaaacaatcatgctttttactctgtaggaatatctttaaaattcttgtgaatttttccccagaagtaaagcaaatcttcccccagaaataaaattaaatgtgcataatctaaagctttttttttttattgtggtaggatatatatataaaacataatttgccattgtaaacattttaaatttacaagtcagaggcattaattacatcacaatgttgtgaaattattactactatttccaaaattttctcatcaccccaaactgaaactctgtaactgttgagcaataacctcattcctgtatctctcccaaccccaggtaacctcaaatctttctttttatctttgagacaaggtctcattctatcactcaggtaggagtgcagtggtgtgatcatagctcattgcagcctcaaaatcctgggctcaagcaatcctccttgagtagctaagactataggcacacattaactgcgcctggctgattttgttttttgtagagatgtggtcttgctatgtttcccatgctggtcttgagttcctggcctcaagcagtccttaagattcatccatgttgtggcatgtgtcagaatttcatttgtttttatgactaaataatattccattgtatgtatatacattttgttcatccatcttctgatgaacactgggatatgtctaccttttggctattgtgaataatgctgcagtaaacattgacataacaagtatgtatttgattgcctgtttctaagttcttttgggtatacatcttgagtagaattgctagataatgtcatgttttatttctcttgtgatttcttcttcgatcccctggttgagtgtgttaatttctacatgtttatgaatttcccactgtttttttgttattgatttccaagttcattccattgtgattagagaagatacttagtatgattttaatgtttttgagaattggtgtgtggcctgatagatggtctgtcctggagaatgttcctcatacacttgagcaaaatatttatcatgctattgttgactgtagttttctatatgtctcttaggtcaaggtggtttacaatgtgttaaggttctctttttttaaaaaaatttttgcacagagtatctttttctatgtgttccatgtatttgtgtctttggagctatagtctcttgtagacagcatatcactatcttgttttgttttgttttttctgtccattctgccaatttctgccttttgattggaaaatttaatccatttgcatttaaagtaattaaggaaggactttcttctaccatttaacacttcttctatatgtcatatacttttttggcccctcatttcctctttatggccttcttttctgtttttttgtagtgaactagtctgattctctttccactcccctttgtgtatatttgttagatgttttatttgtggttgctatggggattatagttaacatcctacacttaaaacaatctaatttaaactgataccaatttaccttcaatagcatacaaaatctctactcctgtaaagctctgcccctgccccccttatgttattgatggcacaaattgcctaataaataatttatagttatttgtatgagtttgtcttttaaatcatttaggaaataaaaagtggagttagaaaacagtatgatagtaatactgacttttatatttgtcaatatatttatcttattttggatccttatttcattatatagatttgagttactgtctagtgcccttccatttcggcccaaaggattcccttatgcatttcttgcagggcaagtctaattgtaataaactccctcagcttttgttttatctgagaatgtcttgatttctcccttatttttgatggataattttgccagatacatgaatttttggtaacagtatttttctttcagcactttaaatatgtcatcccactaccttctgacttcatggtttctcatgagatattagatgttataaaatttgaggattcctcattcttgatgagtcagttctgtcttattgcttttcggatttgctcagcttttgtcttttgacagtttgattataacgcggctcagtgtgggtctctgagtttatcccacttagagtttgttgagtttcttggagtcatagatttatgtcttttatcaaattttggacatatttggctattatttcttcaatttttttcactgcttctttcttttccttctgaaatattcttaatgtatatgttggtctgtttgatgctgtctcaccagtttcttaggctgtgttctcttttgttcctcagacttgattattgcagttgcccttctttttatttttttcaagtttgttgattcttctccctgttcagatcaactgttgaactcctctagtgaatttatttcagttactgtacttttcagctccaagatttatctttggttcctttttataacgtctgtgtctttattgatattctcattttgttcatatgtctctttcttcctttagttctttgtccatgttttcctttagctctttgggcttatttaagacaattgtttaaagtctttgcatagtaagtccaatgtctgtgtttcttcagggatggttttcattattttgttttcaatgagccatactttcctgtgtctttgtatgctgtctttttgttgttgaaaactgtatgtttgaacatcataacgtggtggccctgaaaatcagatattccccccttcctgagagttagttttatttttattattgaagattgtagcagtctattgctacatgtgcagtcatttccaaactatttttgcaaagactgtattccttctgtgtgtcatcactgaagtctctgttccttagtttgtgtttaatagtttgacatagatttccttgaaaggagttaaaactagcagaaaaatctctctcccagtctttccagtctttgtagattggttctgtgctgggcttttccattaatacttagccaggcttgtactgagcctaacaatcaggcccaaaagcgtagggtctttgcagatcttgtctgagcatgcttcttgctgtgtatgcacgtagttttctaaatctccctgtatgtgctgttgaatattctaatttcccaaagaaactcctttgcagctttttctcacagaacatagatggttttttggatatcttgaccatagtctttcgacccaggtgtttgcggttgttagttcaccttacacttttttcaagcattgcctactgcttacgatgagtgctctgtcaatcctttaagtagccccagacaggctaccagagacttaaacaagaatttgtaagttctgctcagcttcctctagaaatggggatcagggtccaagacagaatgcagttgctgatttcaagactgctgcaacaccagggagcttgtgggggaagggcaagcagaaatgtcacaaagctttcttgccattttaaagttgcctgttcttgactcagcatttgcttcattgctataaactttttactgtttttcagagttctgataaaattggctatgcctgttcctgctttaaaaaatatatatatattttttagggattggggtctcactatactgaccaggctggtcttgaacttctggcctcaagccatcctctcatttcagcttcccaaagtgctgcaattacacgcgtgaaccaccacacccagcccctgcttgtttttcaatgtgcctactccaccatgttgctcaagtatgtatattttctaaactaccttgtagtgttgtgatgggaaataaatccctgagccttttgaataactcagagagatcaaaaacttagtttatcctattcgaaggattagaaaaatgatatatctttcactttttcagggataggctcctcattagaaggctcctatgtgccgatgctgtacaagacatttcatttctcttaatgtttacaacaagcttgttgccaaggctgatcttgaactcctggcctcaaacgatcctcccagctcagtctcacaaagtgttgggatgtctggccaactaatgactatcttaactcttgtgtttcaatgtttatgccttcttttatcttgactgattgtatgactatgtcttctagaacaatgttgaacagaaatggtgagagcagacatccttgctttaatatttcaccattatatatgatgttaggtatagatttttctcacagatgccttttatcagattgaggaatttatattcctactttgccgaaaggtttttgtagtatgagggggtgctgaattttgtcaaacactttttcggtaataattgagatgattggttctgcagtcatcgagatgtggattttctcctttattctgttcgtgagtgattacactggttgactaatgttaaaacaaccttactttccaggaataaaccctattatcttttttataca

SEQ ID NO: 16 - DNA, 1821 bp, Homo sapiens
  source 1..1821: mol_type = other DNA
tgcgggtacggacagcgcatgagcttatgttgagggcggagcccagaccagcccttcgtcctatcctgcccttccagcacctctcagccgtaacttaaactacacttcccagaagcctcctcagccagggacttccgttgtcgtcagcggaagcggtgacagatcatcccaggccacacagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaatccatttaatttcttcttagaagtttaactcctttggagaatgtgggccttgaataggatcctctgggtccctcttaatctgacagatgagcagacgaggtgcatggcctgggttgcagcttgagagaaccaaaatattcaaaccagatgacttccaaaatgtggggaaagggatggaaaatgaacctgagatggagtccttaatcacgggataaagccctgtgcatctccctcatttcctacaggtaaaagacagtaaagaaattcaggtcacaggccttgggagttcataggaaggagatgtccagtgctgtccagtagaacttt

SEQ ID NO: 17 - DNA, 5159 bp, Homo sapiens
  source 1..5159: mol_type = other DNA
ggtcccggaagtgcgccagtcgtaccttcgcggccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaatcagttaaataagacttgtctcggtcacctgtgccctgtccagactcgaggcagtggtaacactgcacagtgctatgtggcttctctttgaggatttttgggttttgtaactaaattcttgctgccctcatactttttatgtattagaatcatattcgtattgcccttttaaaacattgggatcctccaaaggcctgccccatgtatttaacagtaatacaggaagcatggcaggcaccatgcaaaccaaggatggatggtgcagtccctgtgtcagtgggcggtggtttcctgctggcctggaatcactcatcacctgattgattggctctgtggtcctgggcaggtgcctcataggtgtgtggatatgatgacgtttctttaaaatgtatgtatttaacaaatacttaattgtattaaggtcatgtaccaaggatttgataaagtttaaataatttactctctacttttatccattttatccattttaactcatgtaatcctcatgtgagtattcctgtttaacacttgagtaaactgaggcacagagaacataagttgcatgccatagtcacacactgtgaaagtgaaaagagaatgtgtgcaaaacacgtcacagtcctggtttctgagtaaaggcaggctgttatctttagaatcaagctatcacagggagataggcaatgctgtgggtgttggaggaaggtgagagcctgttgctaacaatttcctggttttaaagctaaggctgattttattgggaagatctcacatgtgtgtggcccctgagagttcccagtgccttttatttgcagtccttccatttggacctcctagctgccccatcaggtcatctccagggctcagaggggtgagaccatttcccaaggtcacagaaccagctctctagtcaccaccctgcctctccctctcacccagagtcagtaccagttttatggctttattacaaactgctgggtccctcccattttcaacttgattgatgggatgtcatcccttatcctgtctgacatttgcctctggcctggttgctagaagtttgccccaggggcaagagttgaaatttggcttcctgaggtgggctttgtggtttgcgtccctaaagtgagcccactactggttgcttgtccatggccaacaccagaaatcccctgagcactacctgggtctcattccaagaaggaagagggtcaggagacctggggagtctcatattccaagttcttctttctttctgggagcagtgggcagttcatggtgttagggcactcacccccacagactggcaaaccctgcaggacttccgtggctgaggctgtgaccggaggccaggaatgccgttgggtggattgtgagtgaatgggccctttgagctgccctctagagagcaaatccagtttcctggagctcctgaatgaatatctgtactggctcgctcagatgcagaagctccattgaccatgaggccttgtgaacatcagtggccacaggcccagtgtgctgcttggcactgcactagtttaggacctgcagcatgtaggtagcgtcctagtgtttataatacaaagctgctctgcacagcttttctgattcttcttgcaatctcctgaggattatctgccccatttttaaaacgaggtggaatacccaaggtcatgtagccagtgagtgctctggaaagccaaagcagctcatcccttcctggggaccacactgctctgctccaccagaccacactatgaaataggaataagtgctcctgttgcaggactgctgggaaaacaggtggtgtgggacttaagtcaccataattttgaagacttgcatgcagagggctccaggaattgtagacattaaggaatttcactttcagttctacccactacttaagtacttgtcatgtactcttagaggaggccagtaatgatcagaaccattttactttaaaattaataatattgtattagagaatatattaaatggttatattgggttatgttaggatatatacttgaatggaaatacatgtactattagcaatcatatttcatttatccctgtaattagacaagaaagcataatatagctctactcatgggtacacataccagtgtataagatttttagaagtttactttttaaaaataaaagcaaaatgtaagatcttaaaaaaaaaaaaaaaaaa

SEQ ID NO: 18 - DNA, 5416 bp, Homo sapiens
  source 1..5416: mol_type = other DNA
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcaggtaattaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 19 - DNA, 1940 bp, Homo sapiens
  source 1..1940: mol_type = other DNA
gagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggaaaaaaaaaaaaaaaaaa

SEQ ID NO: 20 - DNA, 1309 bp, Homo sapiens
  source 1..1309: mol_type = other DNA
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa

SEQ ID NO: 21 - DNA, 2321 bp, Homo sapiens
  source 1..2321: mol_type = other DNA
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 22 - DNA, 1229 bp, Homo sapiens
  source 1..1229: mol_type = other DNA
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 23 - DNA, 5234 bp, Homo sapiens
  source 1..5234: mol_type = other DNA
agagcgtcgggatatcgggtggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaa

SEQ ID NO: 24 - DNA, 1869 bp, Homo sapiens
  source 1..1869: mol_type = other DNA
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 25 - DNA, 2288 bp, Homo sapiens
  source 1..2288: mol_type = other DNA
ggggccgaacgtggtataaaaggggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagagtgttgatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactgggcatggtggctcactgtctgtaatgtattacctgaggcagaagaccacctgagggtaggagtcaagatcagcctgggcaacatagtgagacgctgtctctacaaaaaataattagcctggcctggtggtgcatgcctagtcctagctgatctggaggctgacgtgggaggattgcttgagcctagagtgagctattatcatgccactgtacagcctgggtgttcacagatcttgtgtctcaaaggtaggcagaggcaggaaaagcaaggagccagaattaagaggttgggtcagtctgcagtgagttcatgcatttagaggtgttcttcaagatgactaatgtcaaaaattgagacatctgttgcggttttttttttttttttttcccctggaatgcagtggcgtgatctcagctcactgcagcctccgcctcctgggttcaagtgattctagtgcctcagcctcctgagtagctgggataatgggcgtgtgccaccatgcccagctaatttttgtatttttagtatagatggggtttcatcattttgaccaggctggtctcaaactcttgacctcagctgatgcgcctgccttggcctcccaaactgctgagattacagatgtgagccaccgcaccctacctcattttctgtaacaaagctaagcttgaacactgttgatgttcttgagggaagcatattgggctttaggctgtaggtcaagtttatacatcttaattatggtggaattcctatgtagagtctaaaaagccaggtacttggtgctacagtcagtctccctgcagagggttaaggcgcagactacctgcagtgaggaggtactgcttgtagcatatagagcctctccctagctttggttatggaggctttgaggttttgcaaacctgaccaatttaagccataagatctggtcaaagggatacccttcccactaaggacttggtttctcaggaaattatatgtacagtgcttgctggcagttagatgtcaggacaatctaagctgagaaaaccccttctctgcccaccttaacagacctctagggttcttaacccagcaatcaagtttgcctatcctagaggtggcggatttgatcatttggtgtgttgggcaatttttgttttactgtctggttccttctgcgtgaattaccaccaccaccacttgtgcatctcagtcttgtgtgttgtctggttacgtattccctgggtgataccattcaatgtcttaatgtacttgtggctcagacctgagtgcaaggtggaaataaacatcaaacatcttttcattatcccta

SEQ ID NO: 26 - DNA, 2439 bp, Homo sapiens
  source 1..2439: mol_type = other DNA
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 27 - DNA, 1196 bp, Homo sapiens
  source 1..1196: mol_type = other DNA
cacttctgccgcccctgtttcaagggataagaaaccctgcgacaaaacctcctccttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 28 - DNA, 987 bp, Homo sapiens
  source 1..987: mol_type = other DNA
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 29 - DNA, 3003 bp, Homo sapiens
  source 1..3003: mol_type = other DNA
ctttctccttccccttcttccgggctcccgtcccggctcatcacccggcctgtggcccactcccaccgccagctggaaccctggggactacgacgtccctcaaaccttgcttctaggagataaaaagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgatttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatcccaaacactgttttgttgcttgttttattatgacctcagattaaattgggaaatattggcccttttgaataattgtcccaaatattacattcaaataaaagtgcaatggagaaaaaaaaaaa

SEQ ID NO: 30 - DNA, 2803 bp, Homo sapiens
  source 1..2803: mol_type = other DNA
actgcagccccgctcgactccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaatccatttgaaatattttactgttgtgactttagtcatatttgttgacctaaaaatcaaatgtaatctttgtattgtgttacatcaaaatccagatattttgtatagtttcttttttctttttcttttcttttttttttttgagacaggatcggtgcagtagtacaatcacagctcactgcagcctcaaactcctgggcagctcaggtgatcttcctgactcagccttctgagtagttggggctacaggtgtgcaccaccatgcccagctcatttattttgtaattgtagggacagggtctcactgtgttgcctaggctggtctcaagtgatcctccctccttggcctcccaaggtgctggaattataggtgtgaacaaaccaaaaaaaaaaaaaa

SEQ ID NO: 31 - DNA, 1435 bp, Homo sapiens
  source 1..1435: mol_type = other DNA
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa